(19) United States
(12) Patent Application Publication
(10) Pub. No.: US 2024/0285649 A1
(43) Pub. Date: Aug. 29, 2024

(54) PROGESTOGEN FORMULATIONS FOR USE IN MODULATING A CYTOKINE STORM MEDIATOR

(71) Applicant: Shenzhen Evergreen Therapeutics Co., Ltd, Shenzhen, Guangdong Province (CN)

(72) Inventors: Xin Du, Shenzhen (CN); Tao Tom Du, Shenzhen (CN); Weiwei Hua, Shenzhen (CN)

(21) Appl. No.: 17/923,539

(22) PCT Filed: May 6, 2021
(86) PCT No.: PCT/US2021/031189

§ 371 Date: Nov. 4, 2022

Related U.S. Application Data
(60) Provisional application No. 63/021,630, filed on May 7, 2020.

(30) Foreign Application Priority Data
Dec. 18, 2020 (CN) 202011506821.4

(51) Int. Cl. A61K 31/57; A61K 9/48; A61K 47/10; A61K 47/14; A61P 11/00; A61P 31/00

(52) U.S. Cl. CPC A61K 31/57; A61K 9/4808; A61K 47/10; A61K 47/14; A61P 11/00; A61P 31/00

(57) ABSTRACT

Disclosed herein, in certain embodiments, are compositions, solutions, and soft gelatin capsules comprising 17-alpha hydroxyprogesterone caproate (17-HPC). In certain embodiments, also disclosed herein are methods, dosing regimens, and kits for use in the treatment of a disease or condition.

### CROSS-REFERENCE TO RELATED PATENT APPLICATION

This application claims priority under 35 U.S.C § 119(e) to U.S. Provisional Patent Application No. 63/021,630, filed May 7, 2020, and CN202011506821.4, filed Dec. 19, 2020, the contents of each of which is hereby incorporated by reference in its entirety.

### BACKGROUND

Cytokine storm or hypercytokinemia, is a severe immune reaction in which the body releases a high level of cytokines into the blood in a time frame which induces high fever, inflammation, severe fatigue, and/or nausea. In some instances, the cytokine storm may be life threatening, leading to multiple organ failure.

Cytokine release syndrome (CRS) is an acute systemic inflammatory syndrome that is characterized by fever, fatigue, headache, rash, arthralgia, and myalgia. In severe cases, the symptoms include uncontrolled systemic inflammatory response, vascular leakage, disseminated intravascular coagulation, and multiple organ failure. In some instances, the CRS is triggered by infections and/or certain drugs. Exemplary drugs that trigger cytokine storms and induces CRS include antibody-based therapies such as anti-thymocyte globulin (ATG), the CD28 superagonist TGN1412, rituximab, obinutuzumab, alemtuzumab, brentuximab, dacetuzumab, and nivolumab; and T cell-engaging immunotherapeutic agents such as bispecific antibody constructs, chimeric antigen receptor (CAR) T cell therapies, and CAR NK cell therapies.

COVID-19 (or SARS-CoV2), a novel coronavirus that is the cause of the pandemic in 2020, has been observed to induce cytokine storm in a subpopulation of patients. In some instances, the infected patients have been observed to have an elevated level of proinflammatory cytokines such as interleukin (IL)-2, IL-4, IL-6, IL-1, IL-17, IP-10, IL-7, tumor necrosis factor as well as G-CSF and M-CSF.

Thus a need exists in the art for safe and effective therapies to mitigate the proinflammatory response to certain infections, drugs and therapies. This disclosure satisfies this need and provides related advantages as well.

### SUMMARY

In certain embodiments, disclosed herein are compositions, solutions, and soft gelatin capsules comprising 17-alpha hydroxyprogesterone caproate (17-HPC), one or more solubilizing agents, and one or more lipophilic agents for use in modulating one or more mediators of a cytokine storm. In some embodiments, also disclosed herein are compositions, solutions, and soft gelatin capsules comprising 17-HPC, one or more solubilizing agents, and one or more lipophilic agents in combination with one or more additional therapeutic agents or additional therapy regimens. In some embodiments, the composition comprises a range of 17-alpha hydroxyprogesterone caproate (17-HPC), one or more solubilizing agents, and one or more lipophilic agents. In some embodiments, the composition comprises a range of 17-alpha hydroxyprogesterone caproate (17-HPC) and a 2-component solvent system. In some instances, the 2-component solvent system comprises a range of solubilizing agent, and a range of lipophilic excipient.

In certain embodiments, disclosed herein is a method of treating a respiratory disease or condition associated with or induced by a pathogen in a subject in need thereof, comprising, or alternatively consisting essentially of, or yet further consisting of administering to the subject a composition comprising 17-alpha hydroxyprogesterone caproate (17-HPC), one or more solubilizing agents, and one or more lipophilic agents to treat the respiratory disease in the subject. In some embodiments, the composition comprises a range of 17-alpha hydroxyprogesterone caproate (17-HPC), one or more solubilizing agents, and one or more lipophilic agents. In some embodiments, the composition comprises a range of 17-alpha hydroxyprogesterone caproate (17-HPC) and a 2-component solvent system. In some instances, the 2-component solvent system comprises a range of solubilizing agent, and a range of lipophilic excipient.

In certain embodiments, also disclosed herein is a method of treating a cytokine release syndrome (CRS) in a subject in need thereof, comprising or alternatively consisting essentially of, or yet further consisting of administering to the subject a composition comprising 17-alpha hydroxyprogesterone caproate (17-HPC), one or more solubilizing agents, and one or more lipophilic agents to treat the cytokine release syndrome in the subject. In some embodiments, the composition comprises a range of 17-alpha hydroxyprogesterone caproate (17-HPC), one or more solubilizing agents, and one or more lipophilic agents. In some embodiments, the composition comprises a range of 17-alpha hydroxyprogesterone caproate (17-HPC) and a 2-component solvent system. In some instances, the 2-component solvent system comprises a range of solubilizing agent, and a range of lipophilic excipient.

In certain embodiments, further disclosed herein is a method of modulating the level of one or more mediators of cytokine storm in a subject in need thereof, comprising, or alternatively consisting essentially of, or yet further consisting of administering to the subject a composition comprising 17-alpha hydroxyprogesterone caproate (17-HPC), one or more solubilizing agents, and one or more lipophilic agents to modulate the level of the one or more mediators. In some embodiments, the composition comprises a range of 17-alpha hydroxyprogesterone caproate (17-HPC), one or more solubilizing agents, and one or more lipophilic agents. In some embodiments, the composition comprises a range of 17-alpha hydroxyprogesterone caproate (17-HPC) and a 2-component solvent system. In some instances, the 2-component solvent system comprises a range of solubilizing agent, and a range of lipophilic excipient.

In certain embodiments, additionally disclosed herein is a method of treating a subject selected for therapy, comprising or alternatively consisting essentially of, or yet further consisting of administering to the subject having an elevated level of a mediator associated with cytokine storm as compared to a predetermined level of the mediator a composition comprising 17-alpha hydroxyprogesterone caproate (17-HPC), one or more solubilizing agents, and one or more lipophilic agents. In some embodiments, the composition comprises a range of 17-alpha hydroxyprogesterone caproate (17-HPC), one or more solubilizing agents, and one or more lipophilic agents. In some embodiments, the composition comprises a range of 17-alpha hydroxyprogesterone caproate (17-HPC) and a 2-component solvent system. In some instances, the 2-component solvent system comprises a range of solubilizing agent, and a range of lipophilic excipient.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates an exemplary flow chart of the manufacturing process for a composition, solution, or soft gelatin capsule comprising 17-HPC.

FIG. 2 illustrates a concentration-time profile of PR2005 following 250 mg intramuscular injection to dogs.

FIG. 3 illustrates a concentration-time profile of PR2005 following 250 mg oral administration to dogs (Powder in capsules).

FIG. 4 illustrates a concentration-time profile of PR2005 following 250 mg oral administration to dogs (Suspension #1).

FIG. 5 illustrates a concentration-time profile of PR2005 following 250 mg oral administration to dogs (Solution #3).

FIG. 6 illustrates a concentration-time profile of PR2005 following 750 mg oral administration to dogs (Solution #3).

FIG. 7 illustrates a concentration-time profile of PR2005 following 250 mg oral administration to dogs (Solution #5).

FIG. 8 illustrates a concentration-time profile of 17-HPC following a 120 mg, 240 mg, 480 mg, and 720 mg oral administration in a human study.

### DETAILED DESCRIPTION

### Definitions

As used in the specification and claims, the singular form “a”, “an”, and “the” include plural references unless the context clearly dictates otherwise. For example, the term “a cell” includes a plurality of cells, including mixtures thereof.

As used herein, the term “comprising” is intended to mean that the compositions or methods include the recited steps or elements, but do not exclude others. “Consisting essentially of” shall mean rendering the claims open only for the inclusion of steps or elements, which do not materially affect the basic and novel characteristics of the claimed compositions and methods. “Consisting of” shall mean excluding any element or step not specified in the claim. Embodiments defined by each of these transition terms are within the scope of this disclosure.

As used herein, the term “about” is used to indicate that a value includes the standard deviation of error for the device or method being employed to determine the value. The term “about” when used before a numerical designation, e.g., temperature, time, amount, and concentration, including range, indicates approximations which may vary by (+) or (−) 15%, 10%, 5%, 3%, 2%, or 1%.

As used herein, the term “mammal” includes both human and non-human mammals.

The term “subject,” “host,” “individual,” and “patient” are as used interchangeably herein to refer to animals, typically mammalian animals. Any suitable mammal can be treated by a method, cell or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human.

As used herein, the term “equivalent in age” in the context of a subject equivalent in age to a human subject 18 years of age or older refers to a non-human subject, for example, a non-human primate, a domestic animal, a farm animal, or an experimental animal in which the age of the subject is equivalent or comparable to the age of a human subject 18 years or older.

As used herein, the term “equivalent in age” in the context of a subject equivalent in age to a human subject 17 years of age or younger refers to a non-human subject, for example, a non-human primate, a domestic animal, a farm animal, or an experimental animal in which the age of the subject is equivalent or comparable to the age of a human subject 17 years of age or younger.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, the term “treatment” excludes prophylaxis.

The term “progestogens” refer to progesterone, a naturally occurring hormone, and progestins, synthetic progesterones or progesterone analogues. Progestins or synthetic progesterones include hydroxyprogesterone esters such as hydroxyprogesterone caproate (or 17-alpha hydroxyprogesterone caproate), hydroxyprogesterone acetate, or hydroxyprogesterone heptanoate.

17-alpha hydroxyprogesterone caproate (17-OHPC or 17-HPC) or 17-[(1-Oxohexyl)oxyl]-pregn-4-ene-3,20-dione, is a synthetic progestogen, derived from 17alpha-hydroxyprogesterone (17-OPC) and caproic acid. The chemical structure of 17-HPC is shown below (MW: 428.6 g/mol and melting point: 119° C.).

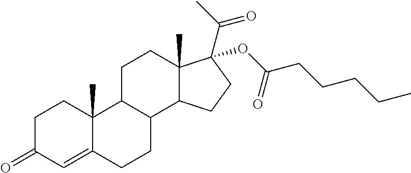 [SMILES: CCCCCC(=O)O[C@]1(C(C)=O)CCC2C3CCC4=CC(=O)CC[C@]4(C)C3CC[C@@]21C]

Hydroxyprogesterone caproate (marketed under the brand name MAKENA®) is an intramuscular injection and is used for the prevention of spontaneous preterm births in singleton pregnancies in women who have previously had a spontaneous preterm birth. In some instances, 17-HPC has been reported to be inactive when administered orally (Saxton D J et al. “Functional effects of 17alpha-hydroxyprogesterone caproate (17P) on human myometrial contractility in vitro,” Reproductive Biology and Endocrinology, 2:80, 2004).

As used herein, the term “solubilizing agent” is an excipient that improves the solubility of a drug in a solution. In some embodiments, the solubilizing agent comprises a surfactant, a compound that lowers the surface tension between two liquids. In some instances, the solubilizing agent comprises a non-aqueous solubilizing agent. Exemplary solubilizing agents include, but are not limited to, benzyl benzoate (CAS 120-51-4), diethylene glycol monoethyl ether, propylene glycol monolaurate, glyceryl monocaprylate, propylene glycol monocaprylate, and oleic acid. In some cases, the solubilizing agent solubilizes 17-HPC, with a solubility of about 75 mg/g, about 120 mg/g, about 150 mg/g, or about 300 mg/g.

As used herein, the term “lipophilic agent” and “lipophilic excipient” are interchangeable and refers to an excipient that includes fatty acids, waxes, sterols, monoglycerides, diglycerides, triglycerides, or phospholipids. Exemplary lipophilic agents include, but are not limited to, macrogolglycerol ricinolate, polysorbate 80, caprylocaproyl polyoxyl-8 glycerides, glyceryl monooleate, glyceryl monolinoleate, PEG 15 hydroxystearate, PEG 40 hydrogenated castor oil, and olive oil.

Macrogolglycerol ricinolate (CAS 61791-12-6) is a mixture of triricinoleate esters of ethoxylated glycerol with small amounts of polyethyleneglycol (macrogol) ricinoleate and the corresponding free glycols. Synonyms of macrogolglycerol ricinolate include polyoxy-35-castor oil; castor oil, ethoxylated, PEG 35 castor oil, KOLLIPHOR®, and CHROMOPHOR®.

Castor oil (CAS 8001-79-4) is a lipophilic agent derived from castor beans.

As used herein, one or more additional excipients comprise a flavoring excipient, a preservative, a diluent, or a combination thereof.

A flavoring excipient comprises natural flavors, natural with other natural flavors, artificial flavors, or natural and artificial flavors. Exemplary flavoring excipients include, but are not limited to, ethyl vanillin, peppermint, oil, almond oil, benzaldehyde, or a bitter masking flavor.

Preservatives comprise additives that reduces or minimizes degradation one or more components of a composition, solution, or soft gelatin capsule described herein. Exemplary preservatives include, but are not limited to, alkyl or aryl alcohols such as benzyl alcohol, or chlorobutanol; amino aryl acid esters such as methyl paraben, ethyl paraben, propyl paraben, or butyl paraben; phenols such as phenol, meta cresol, or chloro cresol; alkyl or aryl acids such as benzoic acid or sorbic acid; organic mercurial such as thiomersal or phenylmercuric nitrate; diols such as bronopol or propylene glycol; or quaternary ammonium compounds such as benzylkonium chloride (BAC) or benzethonium chloride. In some instances, the preservatives comprise an antimicrobials. Antimicrobials include, but are not limited to, methyl paraben, ethyl paraben, propyl paraben, butyl paraben, sorbic acid, sodium sorbate, potassium sorbate, calcium sorbate, benzoic acid, sodium benzoate, potassium benzoate, or calcium benzoate, sodium metabisulfite, propylene glycol, BHT, BHA, benzaldehyde, benzylkonium chloride (BAC) or benzethonium chloride.

Diluents are fillers to include weight and/or improve content uniformity. Exemplary diluents include starches, hydrolyzed starches, partially pregelatinized starches, anhydrous lactose, lactose monohydrate, or sugar alcohols such as sorbitol, xylitol, or mannitol.

Micronization is a process of reducing the diameter of a solid material's particle to enable solubility of the material. Methods of preparing a micronized material are well-known in the art. Exemplary methods include “bottom-up” approaches such as crystallization, spray drying, ionic complexation, and precipitation of dissolved active pharmaceuticals; and “top down” approach involves the mechanical reduction of previously formed larger particles to the desired size. The mechanical reduction process relies on milling and/or grinding and includes techniques such as wet milling, dry milling, ball/pearl milling, spiral media milling, jet milling, high pressure homogenization (HPH), or any other form of impact milling known in the art.

In some instances, 17-HPC is micronized by a method of milling the bulk material using a number of cycles of high pressure homogenization wherein the number of cycles is sufficient to reduce the bulk material to a fine particulate, wherein the fine particulate has a Dv50 of at least less than 57 μm and a span distribution value of less than 6. In some instances, the high pressure homogenization is water jet milling. In some cases, the number of cycles is at least 25.

As used herein, the term “mediator” refers to a protein or peptide associated with or induces a cytokine storm in a subject. In some instances, the level of the mediator (e.g., a serum level or an expression level) is elevated compared to a predetermined level in the subject, and the elevated level is associated with or induces the cytokine storm.

In some embodiments, a subject having a cytokine storm is characterized with an elevated level of one or more mediators. In some cases, the one or more mediators comprise a proinflammatory cytokine. In some instances, the one or more mediators comprise an anti-inflammatory cytokine. In some cases, the one or more mediators comprises a cytokine produced by a Th1, Th2, or Th17 cell. In some cases, the one or more mediators comprises an interleukin, an interferon, a chemokine, or a tumor necrosis factor.

In some embodiments, the one or more mediators comprise an interleukin. Exemplary interleukins include, but are not limited to, IL-1A, IL-1B, IL-2, IL-3, IL-4, IL-6, IL-9, IL-12, IL-12B, IL-13, IL-16, IL-17F, IL-18BP, IL-21, IL-22, IL-23, IL-28B, leptin, or ciliary neurotrophic factor (CNTF). In some cases, the one or more mediators comprise IL-1A, IL-1B, IL-2, IL-3, IL-4, IL-6, IL-9, IL-12, IL-12B, IL-13, IL-16, IL-17F, IL-18BP, IL-21, IL-22, IL-23, IL-28B, leptin, or ciliary neurotrophic factor (CNTF).

In some embodiments, the one or more mediators comprise an interferon. Exemplary interferons include, but are not limited to, IFNA10, IFN-α7, IFN-α 4-Fc, IFNβ, IFNAα4, IFNγ, IFNα5, or IFNω. In some cases, the one or more mediators comprise a Type I IFN, a Type II IFN, or a Type III IFN. In some cases, the one or more mediators comprise IFNA10, IFN-α7, IFNa 4-Fc, IFNβ, IFNAα4, IFNγ, IFNα5, or IFNω.

In some embodiments, the one or more mediators comprise a chemokine. In some instances, the chemokine comprises a CC chemokine (or a β-chemokine), a CXC chemokine, a C chemokine, or a CX3C chemokine. Exemplary chemokines include, but are not limited to, CCL1, CCL2, CCL3, CCL4, CCL5 (RANTES), CCL7, CCL8, CCL11 (eotaxin), CCL13, CCL17, CCL22, CCL24, CCL26, CXCL8, CXCL9, CXCL10, or CXCL11. In some cases, the one or more mediators comprise CCL1, CCL2, CCL3, CCL4, CCL5 (RANTES), CCL7, CCL8, CCL11 (eotaxin), CCL13, CCL17, CCL22, CCL24, CCL26, CXCL8, CXCL9, CXCL10, or CXCL11.

In some embodiments, the one or more mediators comprise a tumor necrosis factor. Exemplary TNF family members include, but are not limited to, TNFα, TNFβ, TNF SF10, B-cell-activating factor (BAFF), CD30 ligand, CD40 ligand, or CD27 ligand. In some cases, the one or more mediators comprise TNFα, TNFβ, TNF SF10, B-cell-activating factor (BAFF), CD30 ligand, CD40 ligand, or CD27 ligand.

In some embodiments, the one or more mediators comprise a proinflammatory cytokine. Exemplary proinflammatory cytokines include, but are not limited to, TNF-α, IL-1α, IL-1β, IL-2, IL-5, IL-6, IL-9, IL-12 (p70), IL-17 (optionally IL-17A), IFN-γ, TGF-β, granulocyte/macrophage-colony-stimulating factor (GM-CSF), macrophage colony stimulating factor (M-CSF), granulocyte-colony stimulating factor (G-CSF), or reactive oxygen species (ROS).

In some embodiments, the proinflammatory cytokine comprises a proinflammatory chemokine. Exemplary proinflammatory chemokines include, but are not limited to, CCL2, CCL3, CCL-5, IL-8, IFN-γ-induced protein 10 (IP-10), cutaneous T-cell-attracting chemokine (CTACK), monokine induced gamma interferon (MIG), hepatocyte growth factor (HGF), macrophage inflammatory protein 1α (MIP-1α), macrophage inflammatory protein 1θ (MIP-1β), monocyte chemoattractant protein-1 (MCP-1), monocyte chematoctic protein-3 (MCP-3), platelet-derived growth factor (PDGF), regulated upon activation normal T cell expressed and secreted (RANTES), or vascular endothelial growth factor (VEGF).

In some embodiments, the one or more mediators comprise an anti-inflammatory cytokine. Exemplary anti-inflammatory cytokines include, but are not limited to, IL-4, IL-10, IL-13, or fibroblast growth factor (FGF).

In some embodiments, the one or more mediators comprise a D-dimer.

In some embodiments, the cytokine storm is associated with or is induced by a pathogen. As utilized herein, the term “associated with or induced by” in the present context refers to the presence or development of a cytokine storm or the presence or development of CRS in a subject that has an infection caused by a pathogen. In some instances, the pathogen induces an immune dysregulation resulting in the development of a cytokine storm in the subject.

In some instances, the pathogen is a virus, a bacterium, a fungus, a protozoan, or a parasite. Exemplary viruses that induce an infection leading to the development of a cytokine storm in a subject include, but are not limited to, DNA viruses such as single-stranded (ss) DNA viruses, double-stranded (ds) DNA viruses, or DNA viruses that contain both ss and ds DNA regions; or RNA viruses such as single-stranded (ss) RNA viruses (e.g., positive-sense RNA viruses or negative-sense RNA viruses), or double-stranded (ds) RNA viruses.

Exemplary dsDNA viruses include viruses from the family: Myoviridae, Podoviridae, Siphoviridae, Alloherpesviridae, Herpesviridae, Malacoherpesviridae, Lipothrixviridae, Rudiviridae, Adenoviridae, Ampullaviridae, Ascoviridae, Asfaviridae, Baculoviridae, Bicaudaviridae, Clavaviridae, Corticoviridae, Fuselloviridae, Globuloviridae, Guttaviridae, Hytrosaviridae, Iridoviridae, Marseilleviridae, Mimiviridae, Nimaviridae, Pandoraviridae, Papillomaviridae, Phycodnaviridae, Plasmaviridae, Polydnaviruses, Polyomaviridae, Poxviridae, Sphaerolipoviridae, and Tectiviridae.

Exemplary ssDNA viruses include viruses from the family: Anelloviridae, Bacillariodnaviridae, Bidnaviridae, Circoviridae, Geminiviridae, Inoviridae, Microviridae, Nanoviridae, Parvoviridae, and Spiraviridae.

Exemplary DNA viruses that contain both ss and ds DNA regions include viruses from the group of pleolipoviruses. In some cases, the pleolipoviruses include Haloarcula hispanica pleomorphic virus 1, Halogeometricum pleomorphic virus 1, Halorubrum pleomorphic virus 1, Halorubrum pleomorphic virus 2, Halorubrum pleomorphic virus 3, and Halorubrum pleomorphic virus 6.

Exemplary dsRNA viruses include viruses from the family: Birnaviridae, Chrysoviridae, Cystoviridae, Endornaviridae, Hypoviridae, Megavirnaviridae, Partitiviridae, Picobirnaviridae, Reoviridae, Rotavirus, and Totiviridae.

Exemplary positive-sense ssRNA viruses include viruses from the family: Alphaflexiviridae, Alphatetraviridae, Alvernaviridae, Arteriviridae, Astroviridae, Barnaviridae, Betaflexiviridae, Bromoviridae, Caliciviridae, Carmotetraviridae, Closteroviridae, Coronaviridae, Dicistroviridae, Flaviviridae, Gammaflexiviridae, Iflaviridae, Leviviridae, Luteoviridae, Marnaviridae, Mesoniviridae, Narnaviridae, Nodaviridae, Permutotetraviridae, Picornaviridae, Potyviridae, Roniviridae, Retroviridae, Secoviridae, Togaviridae, Tombusviridae, Tymoviridae, and Virgaviridae.

Exemplary negative-sense ssRNA viruses include viruses from the family: Arenaviridae, Bornaviridae, Bunyaviridae, Filoviridae, Nyamiviridae, Ophioviridae, Orthomyxoviridae, Paramyxoviridae, and Rhabdoviridae.

In some embodiments, the virus that induces an infection leading to the development of a cytokine storm in a subject is a member of the coronaviruses. Coronaviruses is a family of single-stranded, positive-strand RNA viruses characterized with crown-like spikes on their surface. The coronaviruses belong to the Coronaviridae family, Nidovirales order. There are four sub-groupings or categories of CoVs, alpha, beta, gamma, and delta. The CoVs are the largest known RNA viruses, comprising 16 non-structural proteins and 4 structural proteins which include spike (S) protein, envelope (E) protein, membrane (M) protein, and nucleocapsid (N) protein.

There are seven species of coronaviruses that are known to cause respiratory and intestinal infections in humans. The seven species are 229E (or α-type HCoV-229E), NL63 (or α-type HCoV-NL63), OC43 (or β-type HCoV-OC43), HKU1 (or β-type HCoV-HKU1), MERS-CoV (the β-type HCoV that causes Middle East Respiratory Syndrome or MERS), SARS-CoV (the β-type HCoV that causes severe acute respiratory syndrome or SARS), and SARS-CoV2 (the β-type HCoV that causes the coronavirus disease of 2019, COVID-19, or 2019-nCoV).

In some embodiments, the CoVs are also classified based on their pathogenicity. In some instances, the mild pathogenic CoVs include HCoV-229E, HCoV-OC43, HCoV-NL63, and HCoV-HKU1. In some instances, the highly pathogenic CoVs include SARS-CoV, MERS-CoV, and SARS-CoV2. In some cases, the mild pathogens infect the upper respiratory tract and causes seasonal, mild to moderate cold-like respiratory diseases in the subject. In some cases, the highly pathogenic CoVs infect the lower respiratory tract and cause severe pneumonia, leading, in some cases, to fatal acute lung injury (ALI) and/or acute respiratory distress syndrome (ARDS).

In some embodiments, the virus that induces an infection leading to the development of a cytokine storm in a subject is a coronavirus. In some instances, the virus is a pathogenic strain. As used herein, the term “pathogenic strain” in the context of a coronavirus encompasses strains that are pathogenic to humans, non-human mammals, or combinations thereof and will be relative to the subject suffering from an infection. For example, a strain of coronavirus may be pathogenic to a human but not a particular simian monkey. Thus, different animal models are contemplated that can be used depending on the host species and the species which does not exhibit CRS or pro-inflammatory symptoms. In some instances, the non-human mammals encompass non-human primates, rodents including mouse and, rat, bovine cat, dog (e.g. beagle), and rabbit. In some instances, the virus is SARS-CoV2.

In some embodiments, the virus that induces an infection leading to the development of a cytokine storm in a subject is an influenza virus, cytomegalovirus, Epstein-Barr virus, variola virus, Ebola, dengue, Measles virus, mumps virus, or rubella virus. In some instances, the influenza virus is influenza A virus.

In some embodiments, the bacterium that induces an infection leading to the development of a cytokine storm in a subject is a Gram-positive bacterium or a Gram-negative bacterium. In some instances, the bacterium comprises a members of the Streptococcus family and forms group A streptococcus (GAS). GAS is a plurality of Gram-positive, beta-hemolytic coccus in chains and causes, e.g., strep throat, skin and soft tissue infections such as impetigo and cellulitis, or toxic shock syndrome (TSS). In some embodiments, the GAS comprises Streptococcus pyogenes or Streptococcus dysgalactiae.

In some instances, the bacterium is Francisella tularensis, Corynebacterium diphtheria, Legionella pneumophila, Streptococcus pneumoniae, Mycobacterium tuberculosis, Bordetella pertussis, Bacillu anthracis, Chlamydia psittaci, Coxiella burnetti, Francisella tularensis, or from the genus Brucella.

In some embodiments, the protozoan that induces an infection leading to the development of a cytokine storm in a subject is Plasmodium falciparum or Entamoeba histolytica.

In some embodiments, the fungus that induces an infection leading to the development of a cytokine storm in a subject is from the genus of Aspergillus, Candida, or Cryptococcus; a member of the Pneumocystis species; a member of Dermatophytosis (also known as ringworm); or a member of Basidiomycota.

In some embodiments, the parasite that induces an infection leading to the development of a cytokine storm comprises a nematode or a trematode. In some cases, the parasite is Echinococcus granulosus, Dirofilaria immitis, Paragonimus westermani, Ascaris lumbricoides, Ancylostoma duodenale, Toxocara canis, Schistosoma mansoni, Schistosoma haematobium, Schistosoma japonicum, Strongyloides stercoralis, Wuchereria bancrofti, or Brugia malayi.

In some embodiments, a respiratory disease or condition is associated with or is induced by a pathogen described herein. In some instances, the respiratory disease or condition comprises a lower respiratory disease or condition. Exemplary lower respiratory diseases or conditions include, but are not limited to, pneumonia, lung abscess, bronchitis (e.g., acute bronchitis), or Whooping cough (pertussis).

In some embodiments, the cytokine storm is associated with or is induced by a non-infectious disease or condition. As used herein, the non-infectious disease or condition is characterized with an elevated level of one or more mediators of the cytokine storm. The term “associated with or induced by” in the context of the non-infectious disease or condition refers to the presence or development of a cytokine storm or the presence or development of CRS in a subject. In some instances, the non-infectious disease or condition is a graft-versus-host disease, pancreatitis, or multiple organ dysfunction syndrome.

As used herein, the term “predetermined level” in the context of a cytokine refers to a range of the level of the cytokine in a control sample. In some instances, the control sample is obtained from a healthy subject, e.g., a subject who does not have one or more of a disease or condition. In other instances, the control sample is obtained from a subject having the disease or condition but has not received treatment with 17-HPC. In such instances, the control sample is taken at a time point prior to administration of the composition comprising 17-HPC or alternatively at different time points during the course of treatment. In some embodiments, the predetermined level is an animal model that is or has symptoms of the disease or condition. In further instances, the control sample is a reference sample specific to the laboratory facility and the predetermined level is established for a particular assay of interest by the laboratory facility that carries out the particular assay of interest. In some cases, the predetermined level is measured utilizing a fluid sample, e.g., a blood, saliva, serum, urine, plasma, tear, synovial fluid, or cerebrospinal fluid sample. In some cases, the predetermined level is a serum level. In some cases, the predetermined level is an expression level. A skilled artisan would appreciate that the level of the cytokine is influenced by the assay, by the subject's age, and by the health of the subject. Methods of measuring a cytokine level are well-known in the art. Exemplary methods include enzyme-linked immunosorbent assay (ELISA), real-time quantitative polymerase chain reaction (PCR), cytokine bead array assays such as the BD™ Cytometric Bead Array (CBA) Kit from BD Biosciences and the Human Cytokine Array from Eve Technologies, or immunohistochemistry (IHC).

An elevated level of a cytokine described herein refers to a level that is higher than the predetermined level of the same cytokine. In some instances, the elevated level of the cytokine is at least 1-fold, 2-fold, 3-fold, 4-fold, 5-fold, 6-fold, 7-fold, 8-fold, 9-fold, 10-fold, 20-fold, 30-fold, 40-fold, 50-fold, 100-fold, 200-fold, 500-fold, or higher than the predetermined level of the same cytokine. In some cases, the elevated level of the cytokine is at least 1-fold or higher than the predetermined level of the same cytokine. In some cases, the elevated level of the cytokine is at least 2-fold or higher than the predetermined level of the same cytokine. In some cases, the elevated level of the cytokine is at least 5-fold or higher than the predetermined level of the same cytokine. In some cases, the elevated level of the cytokine is at least 10-fold or higher than the predetermined level of the same cytokine. In some cases, the elevated level of the cytokine is at least 50-fold or higher than the predetermined level of the same cytokine. In some cases, the elevated level of the mediator is at least 100-fold or higher than the predetermined level of the same mediator.

As used herein, the term “predetermined level” in the context of a mediator associated with cytokine storm refers to a range of the level of the mediator in a control sample. In some instances, the control sample is obtained from a healthy subject, e.g., a subject who does not have one or more of an underlying disease or condition, a respiratory disease or condition due to a pathogen, or a CRS associated with the underlying disease or condition or the respiratory disease or condition. In other instances, the control sample is obtained from a subject having an underlying disease or condition but does not have the respiratory disease or condition. In some embodiments, the predetermined level is an animal model that is or has symptoms of the disease or condition. In additional instances, the control sample is obtained from the subject having a cytokine storm or CRS and will be receiving a composition comprising 17-alpha hydroxyprogesterone caproate (17-HPC). In such instances, the control sample is taken at a time point prior to administration of the composition comprising 17-HPC or alternatively at different time points during the course of treatment. In further instances, the control sample is a reference sample specific to the laboratory facility and the predetermined level is established for a particular assay of interest by the laboratory facility that carries out the particular assay of interest. In some cases, the predetermined level is measured utilizing a fluid sample, e.g., a blood, saliva, serum, urine, plasma, tear, synovial fluid, or cerebrospinal fluid sample. In some cases, the predetermined level is a serum level. In some cases, the predetermined level is an expression level. A skilled artisan would appreciate that the level of the mediator is influenced by the assay, by the subject's age, and by the health of the subject. Methods of measuring a mediator level are well-known in the art. Exemplary methods include enzyme-linked immunosorbent assay (ELISA), real-time quantitative polymerase chain reaction (PCR), cytokine bead array assays such as the BD™ Cytometric Bead Array (CBA) Kit from BD Biosciences and the Human Cytokine Array from Eve Technologies, or immunohistochemistry (IHC).

An elevated level of a mediator described herein refers to a level that is higher than the predetermined level of the same mediator. In some instances, the elevated level of the mediator is at least 1-fold, 2-fold, 3-fold, 4-fold, 5-fold, 6-fold, 7-fold, 8-fold, 9-fold, 10-fold, 20-fold, 30-fold, 40-fold, 50-fold, 100-fold, 200-fold, 500-fold, or higher than the predetermined level of the same mediator. In some cases, the elevated level of the mediator is at least 1-fold or higher than the predetermined level of the same mediator. In some cases, the elevated level of the mediator is at least 2-fold or higher than the predetermined level of the same mediator. In some cases, the elevated level of the mediator is at least 5-fold or higher than the predetermined level of the same mediator. In some cases, the elevated level of the mediator is at least 10-fold or higher than the predetermined level of the same mediator. In some cases, the elevated level of the mediator is at least 50-fold or higher than the predetermined level of the same mediator. In some cases, the elevated level of the mediator is at least 100-fold or higher than the predetermined level of the same mediator.

IL-6 is a pleiotropic cytokine with both anti-inflammatory and proinflammatory properties. Classic IL-6 signaling involves binding to cell-associated gp130 (CD130) and IL-6 receptor (IL-6R) (CD126). IL-6R is expressed on hepatocytes, macrophages, neutrophils, CD4+ T-cells, and podocytes. When IL-6 level is elevated, soluble IL-6R (sIL-6R) initiates a trans-signaling. This occurs when the IL-6/sIL-6R complex activates gp130 on a wider array of cells. Anti-inflammatory properties of IL-6 is associated with classic IL-6 signaling and proinflammatory properties of IL-6 is associated with IL-6 trans-signaling.

In some instances, the predetermined level of IL-6 is determined based on a fluid sample. In some cases, the fluid sample is a blood, saliva, serum, urine, plasma, synovial fluid, or cerebrospinal fluid sample. In some instances, the predetermined level of IL-6 is determined from a control sample. In some instances, the control sample is obtained from a healthy subject, e.g., a subject who does not have an underlying disease or condition, a respiratory disease or condition due to a pathogen, or a CRS associated with the underlying disease or condition or the respiratory disease or condition. In other instances, the control sample is obtained from a subject having an underlying disease or condition but does not have the respiratory disease or condition. In additional instances, the control sample is obtained from the subject having a cytokine storm or CRS and will be receiving a composition comprising 17-alpha hydroxyprogesterone caproate (17-HPC). In such instances, the control sample is taken at a time point prior to administration of the composition comprising 17-HPC. In further instances, the control sample is a reference sample specific to the laboratory facility and the predetermined level is established for a particular assay of interest by the laboratory facility that carries out the particular assay of interest.

Methods of measuring IL-6 level are well-known in the art. In some instances, the methods include IL-6 serum assay from LabCorp (Test: 140916); IL-6 assay from ARUP Laboratories (Test: 0051537) with a predetermined level of 5 pg/mL or less or 2 pg/mL or less (becomes effective May 18, 2020); or IL-6 plasma assay from Mayo Clinic Laboratories with a predetermined level of 1.8 pg/mL or less. Other methods of measuring IL-6 level include real-time quantitative polymerase chain reaction (PCR), cytokine bead array assays such as the BD™ Cytometric Bead Array (CBA) Kit from BD Biosciences and the Human Cytokine Array from Eve Technologies, or immunohistochemistry (IHC). A skilled artisan would appreciate that the IL-6 level is influenced by the assay, by the subject's age, and by the health of the subject.

In some instances, an elevated level of IL-6 refers to a level that is higher than the predetermined level of IL-6. In some instances, the elevated level of IL-6 is at least 1-fold, 2-fold, 3-fold, 4-fold, 5-fold, 6-fold, 7-fold, 8-fold, 9-fold, 10-fold, 20-fold, 30-fold, 40-fold, 50-fold, 100-fold, 200-fold, 500-fold, or higher than the predetermined level of IL-6. In some cases, the elevated level of IL-6 is at least 1-fold or higher than the predetermined level of IL-6. In some cases, the elevated level of IL-6 is at least 2-fold or higher than the predetermined level of IL-6. In some cases, the elevated level of IL-6 is at least 5-fold or higher than the predetermined level of IL-6. In some cases, the elevated level of IL-6 is at least 10-fold or higher than the predetermined level of IL-6. In some cases, the elevated level of IL-6 is at least 50-fold or higher than the predetermined level of IL-6. In some cases, the elevated level of IL-6 is at least 100-fold or higher than the predetermined level of IL-6.

IL-1 represents a group of proinflammatory cytokine produced by multiple cell types such as epithelial cells, macrophages, dendritic cells, endothelial cells, and B cells. IL-1 members include IL-1α and IL-1β. In some instances, the predetermined level of IL-1 (e.g., IL-1α and/or IL-1β) is determined based on a fluid sample. In some cases, the fluid sample is a blood, saliva, serum, urine, plasma, synovial fluid, or cerebrospinal fluid sample. In some instances, the predetermined level of IL-1 (e.g., IL-1α and/or IL-1β) is determined from a control sample. In some instances, the control sample is obtained from a healthy subject, e.g., a subject who does not have an underlying disease or condition, a respiratory disease or condition due to a pathogen, or a CRS associated with the underlying disease or condition or the respiratory disease or condition. In other instances, the control sample is obtained from a subject having an underlying disease or condition but does not have the respiratory disease or condition. In additional instances, the control sample is obtained from the subject having a cytokine storm or CRS and will be receiving a composition comprising 17-alpha hydroxyprogesterone caproate (17-HPC). In such instances, the control sample is taken at a time point prior to administration of the composition comprising 17-HPC. In further instances, the control sample is a reference sample specific to the laboratory facility and the predetermined level is established for a particular assay of interest by the laboratory facility that carries out the particular assay of interest.

Methods of measuring IL-1 level (e.g., IL-1α and/or IL-1β level) are well-known in the art. In some instances, the methods include IL-1β assay from Quest Diagnostics (Test: 1757) with a predetermined level of 3.9 pg/mL or less; IL-1β assay from ARUP Laboratories (Test: 0051536) with a predetermined level of 36 pg/mL or less or 6.7 pg/mL or less (becomes effective May 18, 2020); Human IL-1 beta/IL-1F2 Quantikine ELISA Kit from R&D Systems; or IL-1 beta human ELISA Kit from ThermoFisher Scientific. Other methods of measuring IL-1 level (e.g., IL-1α and/or IL-1β level) include real-time quantitative polymerase chain reaction (PCR), cytokine bead array assays such as the BD™ Cytometric Bead Array (CBA) Kit from BD Biosciences and the Human Cytokine Array from Eve Technologies, or immunohistochemistry (IHC). A skilled artisan would appreciate that the IL-1 level (e.g., IL-1α and/or IL-1β level) is influenced by the assay, by the subject's age, and by the health of the subject.

In some instances, an elevated level of IL-1 (e.g., IL-1α and/or IL-1β) refers to a level that is higher than the predetermined level of IL-1 (e.g., IL-1α and/or IL-1β). In some instances, the elevated level of IL-1 (e.g., IL-1α and/or IL-1β) is at least 1-fold, 2-fold, 3-fold, 4-fold, 5-fold, 6-fold, 7-fold, 8-fold, 9-fold, 10-fold, 20-fold, 30-fold, 40-fold, 50-fold, 100-fold, 200-fold, 500-fold, or higher than the predetermined level of IL-1 (e.g., IL-1α and/or IL-1β). In some cases, the elevated level of IL-1 (e.g., IL-1α and/or IL-1β) is at least 1-fold or higher than the predetermined level of IL-1 (e.g., IL-1α and/or IL-1β). In some cases, the elevated level of IL-1 (e.g., IL-1α and/or IL-1β) is at least 2-fold or higher than the predetermined level of IL-1 (e.g., IL-1α and/or IL-1β). In some cases, the elevated level of IL-1 (e.g., IL-1α and/or IL-1β) is at least 5-fold or higher than the predetermined level of IL-1 (e.g., IL-1α and/or IL-1β). In some cases, the elevated level of IL-1 (e.g., IL-1α and/or IL-1β) is at least 10-fold or higher than the predetermined level of IL-1 (e.g., IL-1α and/or IL-1β). In some cases, the elevated level of IL-1 (e.g., IL-1α and/or IL-1β) is at least 50-fold or higher than the predetermined level of IL-1 (e.g., IL-1α and/or IL-1β). In some cases, the elevated level of IL-1 (e.g., IL-1α and/or IL-1β) is at least 100-fold or higher than the predetermined level of IL-1 (e.g., IL-1α and/or IL-1β).

IL-2 is a proinflammatory cytokine secreted by Th-1 cells. In some instances, the predetermined level of IL-2 is determined based on a fluid sample. In some cases, the fluid sample is a blood, saliva, serum, urine, plasma, synovial fluid, or cerebrospinal fluid sample. In some instances, the predetermined level of IL-2 is determined from a control sample. In some instances, the control sample is obtained from a healthy subject, e.g., a subject who does not have an underlying disease or condition, a respiratory disease or condition due to a pathogen, or a CRS associated with the underlying disease or condition or the respiratory disease or condition. In other instances, the control sample is obtained from a subject having an underlying disease or condition but does not have the respiratory disease or condition. In additional instances, the control sample is obtained from the subject having a cytokine storm or CRS and will be receiving a composition comprising 17-alpha hydroxyprogesterone caproate (17-HPC). In such instances, the control sample is taken at a time point prior to administration of the composition comprising 17-HPC. In further instances, the control sample is a reference sample specific to the laboratory facility and the predetermined level is established for a particular assay of interest by the laboratory facility that carries out the particular assay of interest.

Methods of measuring IL-2 level are well-known in the art. In some instances, the methods include IL-2 serum assay from LabCorp (Test: 140912); IL-2 assay from ARUP Laboratories (Test: 0051588) with a predetermined level of 12 pg/mL or less or 2.1 pg/mL or less (becomes effective May 18, 2020); or Human IL-2 Quantikine ELISA Kit from R&D Systems. Other methods of measuring IL-2 level include real-time quantitative polymerase chain reaction (PCR), cytokine bead array assays such as the BD™ Cytometric Bead Array (CBA) Kit from BD Biosciences and the Human Cytokine Array from Eve Technologies, or immunohistochemistry (IHC). A skilled artisan would appreciate that the IL-2 level is influenced by the assay, by the subject's age, and by the health of the subject.

In some instances, an elevated level of IL-2 refers to a level that is higher than the predetermined level of IL-2. In some instances, the elevated level of IL-2 is at least 1-fold, 2-fold, 3-fold, 4-fold, 5-fold, 6-fold, 7-fold, 8-fold, 9-fold, 10-fold, 20-fold, 30-fold, 40-fold, 50-fold, 100-fold, 200-fold, 500-fold, or higher than the predetermined level of IL-2. In some cases, the elevated level of IL-2 is at least 1-fold or higher than the predetermined level of IL-2. In some cases, the elevated level of IL-2 is at least 2-fold or higher than the predetermined level of IL-2. In some cases, the elevated level of IL-2 is at least 5-fold or higher than the predetermined level of IL-2. In some cases, the elevated level of IL-2 is at least 10-fold or higher than the predetermined level of IL-2. In some cases, the elevated level of IL-2 is at least 50-fold or higher than the predetermined level of IL-2. In some cases, the elevated level of IL-2 is at least 100-fold or higher than the predetermined level of IL-2.

IL-4 is a pleiotropic anti-inflammatory cytokine that functions by suppressing the pro-inflammatory milieu. IL-4 is produced by activated T cells, mast cells, basophils, eosinophils, and Nature Killer T (NKT) cells. In some instances, the predetermined level of IL-4 is determined based on a fluid sample. In some cases, the fluid sample is a blood, saliva, serum, urine, plasma, synovial fluid, or cerebrospinal fluid sample. In some instances, the predetermined level of IL-4 is determined from a control sample. In some instances, the control sample is obtained from a healthy subject, e.g., a subject who does not have an underlying disease or condition, a respiratory disease or condition due to a pathogen, or a CRS associated with the underlying disease or condition or the respiratory disease or condition. In other instances, the control sample is obtained from a subject having an underlying disease or condition but does not have the respiratory disease or condition. In additional instances, the control sample is obtained from the subject having a cytokine storm or CRS and will be receiving a composition comprising 17-alpha hydroxyprogesterone caproate (17-HPC). In such instances, the control sample is taken at a time point prior to administration of the composition comprising 17-HPC. In further instances, the control sample is a reference sample specific to the laboratory facility and the predetermined level is established for a particular assay of interest by the laboratory facility that carries out the particular assay of interest.

Methods of measuring IL-4 level are well-known in the art. In some instances, the methods include IL-4 serum assay from LabCorp (Test: 140914); IL-4 assay from ARUP Laboratories (Test: 0051532) with a predetermined level of 5 pg/mL or less or 2.2 pg/mL or less (becomes effective May 18, 2020); IL-4 Human ELISA Kit from ThermoFisher Scientific; or U-PLEX Human IL-4 assay from Meso Scale Diagnostics. Other methods of measuring IL-4 level include real-time quantitative polymerase chain reaction (PCR), cytokine bead array assays such as the BD™ Cytometric Bead Array (CBA) Kit from BD Biosciences and the Human Cytokine Array from Eve Technologies, or immunohistochemistry (IHC). A skilled artisan would appreciate that the IL-4 level is influenced by the assay, by the subject's age, and by the health of the subject.

In some instances, an elevated level of IL-4 refers to a level that is higher than the predetermined level of IL-4. In some instances, the elevated level of IL-4 is at least 1-fold, 2-fold, 3-fold, 4-fold, 5-fold, 6-fold, 7-fold, 8-fold, 9-fold, 10-fold, 20-fold, 30-fold, 40-fold, 50-fold, 100-fold, 200-fold, 500-fold, or higher than the predetermined level of IL-4. In some cases, the elevated level of IL-4 is at least 1-fold or higher than the predetermined level of IL-4. In some cases, the elevated level of IL-4 is at least 2-fold or higher than the predetermined level of IL-4. In some cases, the elevated level of IL-4 is at least 5-fold or higher than the predetermined level of IL-4. In some cases, the elevated level of IL-4 is at least 10-fold or higher than the predetermined level of IL-4. In some cases, the elevated level of IL-4 is at least 50-fold or higher than the predetermined level of IL-4. In some cases, the elevated level of IL-4 is at least 100-fold or higher than the predetermined level of IL-4.

IL-17 comprises a family of cytokines that include IL-17A, IL-17B, IL-17C, IL-17D, IL-17E (also known as IL-25), and IL-17F. IL-17A is a proinflammatory cytokine produced by T helper cells (or Th17 cells) in response to stimulation with IL-23. In some instances, the predetermined level of IL-17 (e.g., IL-17A) is determined based on a fluid sample. In some cases, the fluid sample is a blood, saliva, serum, urine, plasma, synovial fluid, or cerebrospinal fluid sample. In some instances, the predetermined level of IL-17 (e.g., IL-17A) is determined from a control sample. In some instances, the control sample is obtained from a healthy subject, e.g., a subject who does not have a disease or condition (e.g., an underlying disease or condition, a respiratory disease or condition due to a pathogen, or a CRS associated with the underlying disease or condition or the respiratory disease or condition). In other instances, the control sample is obtained from a subject having the disease or condition (e.g., an underlying disease or condition but does not have a respiratory disease or condition) but has not received treatment with 17-HPC. In such instances, the control sample is taken at a time point prior to administration of the composition comprising 17-HPC. In further instances, the control sample is a reference sample specific to the laboratory facility and the predetermined level is established for a particular assay of interest by the laboratory facility that carries out the particular assay of interest.

Methods of measuring IL-17 (e.g., IL-17A) level are well-known in the art. In some instances, the methods include IL-17 serum assay from Quest Diagnostics (Test: 36625); IL-17 assay from ARUP Laboratories (Test: 2013113) with a predetermined level of 13 pg/mL or less or 1.4 pg/mL or less (becomes effective May 18, 2020); or Human IL-17 Quantikine ELISA Kit from R&D Systems. Other methods of measuring IL-17 (e.g., IL-17A) level include real-time quantitative polymerase chain reaction (PCR), cytokine bead array assays such as the BD™ Cytometric Bead Array (CBA) Kit from BD Biosciences and the Human Cytokine Array from Eve Technologies, or immunohistochemistry (IHC). A skilled artisan would appreciate that the IL-17 (e.g., IL-17A) level is influenced by the assay, by the subject's age, and by the health of the subject.

In some instances, an elevated level of IL-17 (e.g., IL-17A) refers to a level that is higher than the predetermined level of IL-17 (e.g., IL-17A). In some instances, the elevated level of IL-17 (e.g., IL-17A) is at least 1-fold, 2-fold, 3-fold, 4-fold, 5-fold, 6-fold, 7-fold, 8-fold, 9-fold, 10-fold, 20-fold, 30-fold, 40-fold, 50-fold, 100-fold, 200-fold, 500-fold, or higher than the predetermined level of IL-17 (e.g., IL-17A). In some cases, the elevated level of IL-17 (e.g., IL-17A) is at least 1-fold or higher than the predetermined level of IL-17 (e.g., IL-17A). In some cases, the elevated level of IL-17 (e.g., IL-17A) is at least 2-fold or higher than the predetermined level of IL-17 (e.g., IL-17A). In some cases, the elevated level of IL-17 (e.g., IL-17A) is at least 5-fold or higher than the predetermined level of IL-17 (e.g., IL-17A). In some cases, the elevated level of IL-17 (e.g., IL-17A) is at least 10-fold or higher than the predetermined level of IL-17 (e.g., IL-17A). In some cases, the elevated level of IL-17 (e.g., IL-17A) is at least 50-fold or higher than the predetermined level of IL-17 (e.g., IL-17A). In some cases, the elevated level of IL-17 (e.g., IL-17A) is at least 100-fold or higher than the predetermined level of IL-17 (e.g., IL-17A).

Tumor necrosis factor alpha (TNF-α), also referred to as TNF, cachexin, or cachectin, is a proinflammatory cytokine. In some instances, the predetermined level of TNF-α is determined based on a fluid sample. In some cases, the fluid sample is a blood, saliva, serum, urine, plasma, synovial fluid, or cerebrospinal fluid sample. In some instances, the predetermined level of TNF-α is determined from a control sample. In some instances, the control sample is obtained from a healthy subject, e.g., a subject who does not have an underlying disease or condition, a respiratory disease or condition due to a pathogen, or a CRS associated with the underlying disease or condition or the respiratory disease or condition. In other instances, the control sample is obtained from a subject having an underlying disease or condition but does not have the respiratory disease or condition. In additional instances, the control sample is obtained from the subject having a cytokine storm or CRS and will be receiving a composition comprising 17-alpha hydroxyprogesterone caproate (17-HPC). In such instances, the control sample is taken at a time point prior to administration of the composition comprising 17-HPC. In further instances, the control sample is a reference sample specific to the laboratory facility and the predetermined level is established for a particular assay of interest by the laboratory facility that carries out the particular assay of interest.

Methods of measuring TNF-α level are well-known in the art. In some instances, the methods include TNF-α assay from LabCorp (Test: 140673); TNF-α assay from ARUP Laboratories (Test: 0051539) with a predetermined level of 22 pg/mL or less or 7.2 pg/mL or less (becomes effective May 18, 2020); or TNF-α Human ELISA Kit from Invitrogen. Other methods of measuring TNF-α level include real-time quantitative polymerase chain reaction (PCR), cytokine bead array assays such as the BD™ Cytometric Bead Array (CBA) Kit from BD Biosciences and the Human Cytokine Array from Eve Technologies, or immunohistochemistry (IHC). A skilled artisan would appreciate that the TNF-α level is influenced by the assay, by the subject's age, and by the health of the subject.

In some instances, an elevated level of TNF-α refers to a level that is higher than the predetermined level of TNF-α. In some instances, the elevated level of TNF-α is at least 1-fold, 2-fold, 3-fold, 4-fold, 5-fold, 6-fold, 7-fold, 8-fold, 9-fold, 10-fold, 20-fold, 30-fold, 40-fold, 50-fold, 100-fold, 200-fold, 500-fold, or higher than the predetermined level of TNF-α. In some cases, the elevated level of TNF-α is at least 1-fold or higher than the predetermined level of TNF-α. In some cases, the elevated level of TNF-α is at least 2-fold or higher than the predetermined level of TNF-α. In some cases, the elevated level of TNF-α is at least 5-fold or higher than the predetermined level of TNF-α. In some cases, the elevated level of TNF-α is at least 10-fold or higher than the predetermined level of TNF-α. In some cases, the elevated level of TNF-α is at least 50-fold or higher than the predetermined level of TNF-α. In some cases, the elevated level of TNF-α is at least 100-fold or higher than the predetermined level of TNF-α.

In some embodiments, method of measuring one or more of IL-6, IL-1 (e.g., IL-1β), IL-4, IL-17 (e.g., IL-17A), and TNF-α comprises a Cytokine Panel from ARUP Laboratories. In some instances, the predetermined levels for the one or more of IL-6, IL-1 (e.g., IL-1β), IL-4, IL-17 (e.g., IL-17A), and TNF-α are illustrated in Table 16.

TABLE 16

| Test | | Predetermined |
|---|---|---|
| Number | Cytokine | Level |
| 0051529 | Interleukin 2 Receptor | 1033 pg/mL or less; or |
| | (CD25), Soluble | 175.3 pg/mL to 858.2 pg/mL* |
| 0051530 | Interleukin 12 | 6 pg/mL or less; or |
| | | 1.9 pg/mL or less* |
| 0051531 | Interferon gamma | 5 pg/mL or less; or |
| | | 4/2 pg/mL or less* |
| 0051532 | Interleukin 4 | 5 pg/mL or less; or |
| | | 2.2 pg/mL or less* |
| 0051533 | Interleukin 5 | 5 pg/mL or less; or |
| | | 2.1 pg/mL or less* |
| 0051534 | Interleukin 10 | 18 pg/mL or less; or |
| | | 2.8 pg/mL or less* |
| 0051535 | Interleukin 13 | 5 pg/mL or less; or |
| | | 2.3 pg/mL or less* |
| 0051536 | Interleukin 1 beta | 36 pg/mL or less; or |
| | | 6.7 pg/mL or less* |
| 0051537 | Interleukin 6 | 5 pg/mL or less; or |
| | | 2.0 pg/mL or less* |
| 0051538 | Interleukin 8 | 5 pg/mL or less; or |
| | | 3.0 pg/mL or less* |
| 0051539 | Tumor Necrosis Factor - alpha | 22 pg/mL or less; or |
| | | 7.2 pg/mL or less* |
| 0051588 | Interleukin 2 | 12 pg/mL or less; or |
| | | 2.1 pg/mL or less* |
| 2013115 | Interleukin 17 | 13 pg/mL or less; or |
| | | 1.4 pg/mL or less* |

*the predetermined level will become effective May 18, 2020

C-reactive protein (CRP) is an acute phase reactant produced by the liver in response to the presence of inflammation. In particular, the production of CRP is stimulated by IL-6 and as such, CRP serves as a surrogate for IL-6 bioactivity. Under a cytokine storm or CRS setting, the CRP level can be utilized to monitor the progression of the cytokine storm or CRS and/or the progress of the treatment.

A CRP test measures the level of CRP in the blood. Methods of measuring the CRP level are well-known in the art and can include fluorescent immune chromatography, colloidal gold method, enzyme-linked immuno-adsorption, and apoliprotein. CRP detection assays can further be divided into conventional CRP assays which includes qualitative, semi-quantitative and quantitative assays; high sensitivity CRP (hsCRP) assays, and cardiac CRP (cCRP) assays. In some instances, a method for CRP detection is as described in PCT Application Publication No. WO2018/29885A1. In some cases, a predetermined level of CRP comprises a range selected from about 0.2 mg/L to about 6.1 mg/L, from about 0.2 mg/L to about 6 mg/L, or from about 0.2 mg/L to about 5 mg/L.

In some instances, an elevated level of CRP refers to a level that is higher than the predetermined level of CRP. In some instances, the elevated level of CRP is at least 1-fold, 2-fold, 3-fold, 4-fold, 5-fold, 6-fold, 7-fold, 8-fold, 9-fold, 10-fold, 20-fold, 30-fold, 40-fold, 50-fold, 100-fold, 200-fold, 500-fold, or higher than the predetermined level CRP. In some cases, the elevated level of CRP is at least 1-fold or higher than the predetermined level of CRP. In some cases, the elevated level of CRP is at least 2-fold or higher than the predetermined level of CRP. In some cases, the elevated level of CRP is at least 5-fold or higher than the predetermined level of CRP. In some cases, the elevated level of CRP is at least 10-fold or higher than the predetermined level of CRP. In some cases, the elevated level of CRP is at least 50-fold or higher than the predetermined level of CRP. In some cases, the elevated level of CRP is at least 100-fold or higher than the predetermined level of CRP.

Ferritin is a serum protein that stores iron and a ferritin test indirectly measures the amount of iron in the body. Methods for determining the level of ferritin are well-known in the art and can include an enzyme immunoassay. In some cases, the enzyme immunoassay includes a solid-phase enzyme immunoassay, a chemiluminescence enzyme immunoassay, or a radioimmunoassay. In some instances, a predetermined level of ferritin (or serum ferritin) for men comprises a range selected from about 12 to about 300 ng/mL, from about 20 to about 300 ng/mL, or from about 20 to about 250 ng/mL. In some instances, a predetermined level of ferritin (or serum ferritin) for women comprises a range selected from about 12 to about 270 ng/mL, from about 12 to about 263 ng/mL, from about 20 to about 200 ng/mL, from about 12 to about 150 ng/mL, or from about 10 to about 120 ng/mL.

In some instances, an elevated level of ferritin refers to a level that is higher than the predetermined level of ferritin. In some instances, the elevated level of ferritin is at least 1-fold, 2-fold, 3-fold, 4-fold, 5-fold, 6-fold, 7-fold, 8-fold, 9-fold, 10-fold, 20-fold, 30-fold, 40-fold, 50-fold, 100-fold, 200-fold, 500-fold, or higher than the predetermined level of ferritin. In some cases, the elevated level of ferritin is at least 1-fold or higher than the predetermined level of ferritin. In some cases, the elevated level of ferritin is at least 2-fold or higher than the predetermined level of ferritin. In some cases, the elevated level of ferritin is at least 5-fold or higher than the predetermined level of ferritin. In some cases, the elevated level of ferritin is at least 10-fold or higher than the predetermined level of ferritin. In some cases, the elevated level of ferritin is at least 50-fold or higher than the predetermined level of ferritin. In some cases, the elevated level of ferritin is at least 100-fold or higher than the predetermined level of ferritin.

Procalcitonin (PCT) is a peptide precursor of the hormone calcitonin, and calcitonin is involved in calcium homeostasis. A procalcitonin test measures the procalcitonin level in the blood. In some instances, an elevated level of procalcitonin is indicative to bacterial infection such as sepsis. Methods for determining the level of procalcitonin are well-known in the art and can include Procalcitonin Human ELISA Kit from ThermoFisher Scientific or an immunoturbidimetric assay from Diazyme. In some instances, a predetermined level of procalcitonin comprises a range selected from less than or about 0.1 ng/mL, less than or about 0.15 ng/mL, or from about 0.1 ng/mL to about 0.49 ng/mL.

In some instances, an elevated level of procalcitonin refers to a level that is higher than the predetermined level of procalcitonin. In some instances, the elevated level of procalcitonin is at least 1-fold, 2-fold, 3-fold, 4-fold, 5-fold, 6-fold, 7-fold, 8-fold, 9-fold, 10-fold, 20-fold, 30-fold, 40-fold, 50-fold, 100-fold, 200-fold, 500-fold, or higher than the predetermined level of procalcitonin. In some cases, the elevated level of procalcitonin is at least 1-fold or higher than the predetermined level of procalcitonin. In some cases, the elevated level of procalcitonin is at least 2-fold or higher than the predetermined level of procalcitonin. In some cases, the elevated level of procalcitonin is at least 5-fold or higher than the predetermined level of procalcitonin. In some cases, the elevated level of procalcitonin is at least 10-fold or higher than the predetermined level of procalcitonin. In some cases, the elevated level of procalcitonin is at least 50-fold or higher than the predetermined level of procalcitonin. In some cases, the elevated level of procalcitonin is at least 100-fold or higher than the predetermined level of procalcitonin.

Neopterin is a catabolic product of guanosine triphosphate and is synthesized by macrophages and/or monocytes upon stimulation by IFN-γ. As such, neopterin serves a biomarker of immune system activation. A neopterin test measures the level of neopterin in the blood. Methods for determining the level of procalcitonin are well-known in the art and can include an enzyme immunoassay method from LabCorp (e.g, Test: 140335) or a neopterin test from Mayo Clinic Laboratories (Test: FNEOS). In some instances, a predetermined level of procalcitonin is less than 2.5 ng/mL.

In some instances, an elevated level of neopterin refers to a level that is higher than the predetermined level of neopterin. In some instances, the elevated level of neopterin is at least 1-fold, 2-fold, 3-fold, 4-fold, 5-fold, 6-fold, 7-fold, 8-fold, 9-fold, 10-fold, 20-fold, 30-fold, 40-fold, 50-fold, 100-fold, 200-fold, 500-fold, or higher than the predetermined level of neopterin. In some cases, the elevated level of neopterin is at least 1-fold or higher than the predetermined level of neopterin. In some cases, the elevated level of neopterin is at least 2-fold or higher than the predetermined level of neopterin. In some cases, the elevated level of neopterin is at least 5-fold or higher than the predetermined level of neopterin. In some cases, the elevated level of neopterin is at least 10-fold or higher than the predetermined level of neopterin. In some cases, the elevated level of neopterin is at least 50-fold or higher than the predetermined level of neopterin. In some cases, the elevated level of neopterin is at least 100-fold or higher than the predetermined level of neopterin.

The S100 proteins are a family of Ca²⁺ binding EF-hand proteins. Family members S100A8 and S100A9 are constitutively expressed in early myeloid lineages and are expressed by a variety of other cells such as platelets, esteoclasts, keratinocytes, and vascular endothelial cells under infectious and inflammatory conditions. A S100 protein test measures the level of S100 proteins in a tissue or cell sample. A S100A8/9 test measures the level of S100A8 or S100A9 concentration in the serum. c S100 proteins are well-known in the art and can include Human S100A8/S100A9 heterodimer Quantikine ELISA Kit from R&D Systems. In some instances, a predetermined level of S100 proteins (e.g., S100A8 and/or S100A9) is determined based on a control sample. In some instances, the control sample is a reference sample specific to the laboratory facility and the predetermined level is established for a particular assay of interest by the laboratory facility that carries out the particular assay of interest.

In some instances, an elevated level of S100 proteins (e.g., S100A8 and/or S100A9) refers to a level that is higher than the predetermined level of S100 proteins (e.g., S100A8 and/or S100A9). In some instances, the elevated level of S100 proteins (e.g., S100A8 and/or S100A9) is at least 1-fold, 2-fold, 3-fold, 4-fold, 5-fold, 6-fold, 7-fold, 8-fold, 9-fold, 10-fold, 20-fold, 30-fold, 40-fold, 50-fold, 100-fold, 200-fold, 500-fold, or higher than the predetermined level of S100 proteins (e.g., S100A8 and/or S100A9). In some cases, the elevated level of S100 proteins (e.g., S100A8 and/or S100A9) is at least 1-fold or higher than the predetermined level of S100 proteins (e.g., S100A8 and/or S100A9). In some cases, the elevated level of S100 proteins (e.g., S100A8 and/or S100A9) is at least 2-fold or higher than the predetermined level of S100 proteins (e.g., S100A8 and/or S100A9). In some cases, the elevated level of S100 proteins (e.g., S100A8 and/or S100A9) is at least 5-fold or higher than the predetermined level of S100 proteins (e.g., S100A8 and/or S100A9). In some cases, the elevated level of S100 proteins (e.g., S100A8 and/or S100A9) is at least 10-fold or higher than the predetermined level of S100 proteins (e.g., S100A8 and/or S100A9). In some cases, the elevated level of S100 proteins (e.g., S100A8 and/or S100A9) is at least 50-fold or higher than the predetermined level of S100 proteins (e.g., S100A8 and/or S100A9). In some cases, the elevated level of S100 proteins (e.g., S100A8 and/or S100A9) is at least 100-fold or higher than the predetermined level of S100 proteins (e.g., S100A8 and/or S100A9).

Adenosine deaminase 2 (ADA2) is expressed in myeloid cells and is involved in the differentiation of macrophages. Methods for determining the level of ADA2 can include an ELISA-based assay or a quantitative polymerase chain reaction (PCR) method. In some cases, an ADA2 test is a test from Viapath, with a predetermined level of from about 6.9 IU/L to about 59.7 IU/L.

In some instances, an elevated level of ADA2 refers to a level that is higher than the predetermined level of ADA2. In some instances, the elevated level of ADA2 is at least 1-fold, 2-fold, 3-fold, 4-fold, 5-fold, 6-fold, 7-fold, 8-fold, 9-fold, 10-fold, 20-fold, 30-fold, 40-fold, 50-fold, 100-fold, 200-fold, 500-fold, or higher than the predetermined level of ADA2. In some cases, the elevated level of ADA2 is at least 1-fold or higher than the predetermined level of ADA2. In some cases, the elevated level of ADA2 is at least 2-fold or higher than the predetermined level of ADA2. In some cases, the elevated level of ADA2 is at least 5-fold or higher than the predetermined level of ADA2. In some cases, the elevated level of ADA2 is at least 10-fold or higher than the predetermined level of ADA2. In some cases, the elevated level of ADA2 is at least 50-fold or higher than the predetermined level of ADA2. In some cases, the elevated level of ADA2 is at least 100-fold or higher than the predetermined level of ADA2.

CD163 is a high affinity scavenger receptor for the hemoglobin-haptoglobin complex and a low affinity scavenger receptor for hemoglobin alone. An elevated level of CD163 in macrophages is indicative of tissues responding to inflammation. A CD163 test measures the soluble CD163 (sCD163) level in a sample, e.g., in a fluid sample such as blood, saliva, serum, urine, or plasma. Methods of measuring CD163 can include an ELISA-based assay such as the Human CD163 Quantikine ELISA Kit from R&D Systems or the CD163 Human ELISA Kit from Thermo Fisher Scientific. In some cases, a predetermined level of CD163 is determined based on a control sample. In some instances, the control sample is a reference sample specific to the laboratory facility and the predetermined level is established for a particular assay of interest by the laboratory facility that carries out the particular assay of interest.

In some instances, an elevated level of CD163 refers to a level that is higher than the predetermined level of CD163. In some instances, the elevated level of CD163 is at least 1-fold, 2-fold, 3-fold, 4-fold, 5-fold, 6-fold, 7-fold, 8-fold, 9-fold, 10-fold, 20-fold, 30-fold, 40-fold, 50-fold, 100-fold, 200-fold, 500-fold, or higher than the predetermined level of CD163. In some cases, the elevated level of CD163 is at least 1-fold or higher than the predetermined level of CD163. In some cases, the elevated level of CD163 is at least 2-fold or higher than the predetermined level of CD163. In some cases, the elevated level of CD163 is at least 5-fold or higher than the predetermined level of CD163. In some cases, the elevated level of CD163 is at least 10-fold or higher than the predetermined level of CD163. In some cases, the elevated level of CD163 is at least 50-fold or higher than the predetermined level of CD163. In some cases, the elevated level of CD163 is at least 100-fold or higher than the predetermined level of CD163.

Fibrinogen is a soluble glycoprotein involved in blood clotting. During a vascular injury, fibrinogen is converted by thrombin to fibrin and then to a fibrin-based blood clot. Fibrinogen is an acute-phase reactant in response to an increase in proinflammatory cytokines including TNF-α, IL-β, and macrophage chemotactic protein-1. Under an infection setting such as a SARS-CoV setting, the level of fibrinogen is decreased while that of its degradation products such as D-dimer are increased. Methods of measuring the fibrinogen level can include an ELISA-based method, and can determine the level of fibrinogen in the blood. A predetermined level of fibrinogen can include a range of from about 2 to about 4 g/L.

In some instances, a decreased level of fibrinogen refers to a level that is lower than the predetermined level of fibrinogen. In some instances, the decreased level of fibrinogen is at least 1-fold, 2-fold, 3-fold, 4-fold, 5-fold, 6-fold, 7-fold, 8-fold, 9-fold, 10-fold, 20-fold, 30-fold, 40-fold, 50-fold, 100-fold, 200-fold, 500-fold, or lower than the predetermined level of fibrinogen. In some cases, the decreased level of fibrinogen is at least 1-fold or lower than the predetermined level of fibrinogen. In some cases, the decreased level of fibrinogen is at least 2-fold or lower than the predetermined level of fibrinogen. In some cases, the decreased level of fibrinogen is at least 5-fold or lower than the predetermined level of fibrinogen. In some cases, the decreased level of fibrinogen is at least 10-fold or lower than the predetermined level of fibrinogen. In some cases, the decreased level of fibrinogen is at least 50-fold or lower than the predetermined level of fibrinogen. In some cases, the decreased level of fibrinogen is at least 100-fold or lower than the predetermined level of fibrinogen.

D-dimer is a degradation product of crosslinked fibrin resulting from plasmin cleavage. In some instances, an increase in the level of D-dimer correlates with a higher mortality rate. Methods of measuring D-dimers can include latex-enhanced immunoturbidimetric immunoassays such as Advanced D-Dimer assay from Dade Behring Diagnostics, Auto Blue 400 assay from Helena Biosciences, Diazyme D-Dimer Assay from Diazyme Laboratories, HemosIL D-Dimer assay from Instrumentation Laboratory, INNOVANCE D-Dimer assay from Siemens AG, MDAW D-Dimer assay from bioMerieux SA, Nordic Red D-dimer from Nordic Biomarker AB, STA Liatest D-Dimer from Diagnostica Stago, Inc., or Tina-quant D-Dimer BM assay from F. Hoffman-La Roche Ltd.; ELISA-based assays such as VIDAS D-Dimer from bioMerieux SA; or microparticle agglutination assays such as TriniLIA D-Dimer from Tcoag Ireland Ltd. In some cases, a predetermined level of D-dimer is determined based on a control sample. In some instances, the control sample is a reference sample specific to the laboratory facility and the predetermined level is established for a particular assay of interest by the laboratory facility that carries out the particular assay of interest.

In some instances, an elevated level of D-dimer refers to a level that is higher than the predetermined level of D-dimer. In some instances, the elevated level of D-dimer is at least 1-fold, 2-fold, 3-fold, 4-fold, 5-fold, 6-fold, 7-fold, 8-fold, 9-fold, 10-fold, 20-fold, 30-fold, 40-fold, 50-fold, 100-fold, 200-fold, 500-fold, or higher than the predetermined level of D-dimer. In some cases, the elevated level of D-dimer is at least 1-fold or higher than the predetermined level of D-dimer. In some cases, the elevated level of D-dimer is at least 2-fold or higher than the predetermined level of D-dimer. In some cases, the elevated level of D-dimer is at least 5-fold or higher than the predetermined level of D-dimer. In some cases, the elevated level of D-dimer is at least 10-fold or higher than the predetermined level of D-dimer. In some cases, the elevated level of D-dimer is at least 50-fold or higher than the predetermined level of D-dimer. In some cases, the elevated level of D-dimer is at least 100-fold or higher than the predetermined level of D-dimer.

As used herein, the term “underlying disease or condition” refers to a pre-existing disease or condition in the subject prior to the development of the cytokine storm, and/or the development of CRS. In the context of a cytokine storm associated with or induced by a pathogen, the underlying disease or condition does not encompass the infection caused by the pathogen. In the context of the cytokine storm associated with or induced by a non-infectious disease or condition, the underlying disease or condition does not encompass the non-infectious disease or condition such as graft-versus-host disease, pancreatitis, or multiple organ dysfunction syndrome. Exemplary underlying disease or conditions include, but are not limited to, hypertension, cardiovascular disease, diabetes, or chronic lung disease.

As used herein, the term “secondary infection” refers to an infection caused by a pathogen that is not the primary source of infection. In some instances, the pathogen is an opportunistic pathogen. An opportunistic infection refers to a non-pathogenic microorganism such as a bacterium, a virus, a fungus, or a protozoan, or a parasite that induces an infection in a subject with an impaired immune system.

Exemplary opportunistic pathogens include, but are not limited to, Aspergillus sp., Candida albicans, Clostridium difficile, Coccidioides immitis, Cryptococcus neoformans, Cryptosporidium, Cytomegalovirus, Geomyces destructans, Histoplasma capsulatum, Isospora belli, Polyomavirus JC polyomavirus, leukoencephalopathy, Human herpesvirus 8 (HHV8) (or Kaposi's sarcoma-associated herpesvirus or KSHV), Legionella pneumophila, Microsporidium, Mycobacterium avium complex (MAC), Mycobacterium tuberculosis, Pneumocystis jirovecii, Pseudomonas aeruginosa, Salmonella, Staphylococcus aureus, Streptococcus pneumoniae, Streptococcus pyogenes, or Toxoplasma gondii.

### 17-Alpha Hydroxyprogesterone Caproate Formulations

Disclosed herein, in certain embodiments, are compositions comprising 17-HPC. In some embodiments, the composition further comprises a 2-component solvent system. In some instance, the 2-component solvent system comprises a solubilizing agent and a lipophilic agent or excipient. In some instances, the composition comprises, consisting essentially or, or consisting of a range of 17-alpha hydroxyprogesterone caproate (17-HPC) and a range of the 2-component solvent system. In some instances, the composition comprises, consisting essentially or, or consisting of a range of 17-alpha hydroxyprogesterone caproate (17-HPC), a range of solubilizing agent, and a range of lipophilic excipient.

In some embodiments, the range of 17-HPC is selected from: about 12% w/w to about 75% w/w, about 12% w/w to about 74% w/w, about 12% w/w to about 63% w/w, about 12% w/w to about 36% w/w, about 12% w/w to about 30% w/w, about 12% w/w to about 25% w/w, about 12% w/w to about 24% w/w, about 24% w/w to about 75% w/w, about 24% w/w to about 74% w/w, about 24% w/w to about 63% w/w, about 24% w/w to about 36% w/w, about 24% w/w to about 30% w/w, about 25% w/w to about 75% w/w, about 25% w/w to about 74% w/w, about 25% w/w to about 63% w/w, about 25% w/w to about 36% w/w, about 25% w/w to about 30% w/w, about 36% w/w to about 75% w/w, about 36% w/w to about 74% w/w, or about 36% w/w to about 63% w/w. In some instances, the range of 17-HPC is from about 24% w/w to about 75% w/w. In some instances, the range of 17-HPC is from about 24% w/w to about 74% w/w. In some instances, the range of 17-HPC is from about 24% w/w to about 63% w/w. In some instances, the range of 17-HPC is from about 24% w/w to about 36% w/w. In some instances, the range of 17-HPC is from about 36% w/w to about 75% w/w. In some instances, the range of 17-HPC is from about 36% w/w to about 74% w/w. In some instances, the range of 17-HPC is from about 36% w/w to about 63% w/w.

In some instances, the range of 17-HPC is selected from: about 12% w/w to about 36% w/w, about 12% w/w to about 25% w/w, about 12% w/w to about 24% w/w, about 24% w/w to about 36% w/w, or about 25% w/w to about 36% w/w. In some instances, the range of 17-HPC is from about 12% w/w to about 36% w/w. In some instances, the range of 17-HPC is from about 12% w/w to about 25% w/w. In some instances, the range of 17-HPC is from about 12% w/w to about 24% w/w. In some instances, the range of 17-HPC is from about 24% w/w to about 36% w/w. In some instances, the range of 17-HPC is from about 25% w/w to about 36% w/w.

In some embodiments, the composition comprises about 12% w/w, about 24% w/w, 25% w/w, about 30% w/w/, about 36% w/w, about 60% w/w, about 63% w/w, about 74% w/w, or about 75% w/w of 17-HPC. In some cases, the composition comprises about 12% w/w, 24% w/w, 25% w/w, or 36% w/w of 17-HPC. In some cases, the composition comprises about 12% w/w of 17-HPC. In some cases, the composition comprises about 24% w/w of 17-HPC. In some cases, the composition comprises about 25% w/w of 17-HPC. In some cases, the composition comprises about 36% w/w of 17-HPC. In some cases, the composition comprises about 74% w/w of 17-HPC. In some cases, the composition comprises about 75% w/w of 17-HPC.

In some embodiments, the range of 17-HPC is selected from: about 6% w/v to about 36% w/v, about 6% w/v to about 24% w/v, about 6% w/v to about 18% w/v, about 12% w/v to about 36% w/v, about 12% w/v to about 18% w/v, about 12% w/v to about 24% w/v, about 18% w/v to about 36% w/v, or about 24% w/v to about 36% w/v.

In some embodiments, the composition comprises a range of the 2-component solvent system. In some instances, the range of the 2-component solvent system is selected from: about 25% w/w to about 88% w/w, about 25% w/w to about 76% w/w, about 25% w/w to about 75% w/w, about 25% w/w to about 36% w/w, about 26% w/w to about 88% w/w, about 26% w/w to about 76% w/w, about 26% w/w to about 75% w/w, about 26% w/w to about 64% w/w, about 64% w/w to about 88% w/w, about 64% w/w to about 76% w/w, about 64% w/w to about 75% w/w, or about 75% w/w to about 88% w/w.

In some cases, the range of the 2-component solvent system is selected from: about 10% w/w to about 90% w/w, about 10% w/w to about 80% w/w, about 10% w/w to about 70% w/w, about 10% w/w to about 60% w/w, about 10% w/w to about 50% w/w, about 15% w/w to about 90% w/w, about 15% w/w to about 80% w/w, about 15% w/w to about 70% w/w, about 15% w/w to about 60% w/w, about 15% w/w to about 50% w/w, about 20% w/w to about 90% w/w, about 20% w/w to about 80% w/w, about 20% w/w to about 70% w/w, about 20% w/w to about 60% w/w, or about 20% w/w to about 50% w/w.

In some cases, the composition comprises about 10% w/w, about 15% w/w, about 2-% w/w, about 25% w/w, about 26% w/w, about 64% w/w, about 75% w/w, about 76% w/w, or about 88% w/w of the 2-component solvent system.

In some embodiments, the 2-component solvent system comprises a solubilizing agent and a lipophilic excipient. In some instances, the solubilizing agent comprises benzyl benzoate, diethylene glycol monoethyl ether, propylene glycol monolaurate, glyceryl monocaprylate, propylene glycol monocaprylate, or oleic acid. In some cases, the solubilizing agent comprises benzyl benzoate. In some instances, the lipophilic excipient comprises macrogolglycerol ricinolate, polysorbate 80, caprylocaproyl polyoxyl-8 glycerides, glyceryl monooleate, glyceryl monolinoleate, PEG 15 hydroxystearate, PEG 40 hydrogenated castor oil, PEG 35 castor oil, or olive oil. In some cases, the lipophilic excipient comprises macrogolglycerol ricinolate. In some cases, the weight (mg) ratio of the solubilizing agent to the lipophilic agent is 9:1. As an illustrative example, in a composition comprising 240 mg of 17-HPC, the solubilizing agent in the composition is 684 mg and the lipophilic agent in the composition is 76 mg. The weight (mg) ratio of the solubilizing agent (684 mg) to the lipophilic agent (76 mg) is 9:1. In some cases, the weight (mg) ratio of the solubilizing agent to the lipophilic agent is 8:2. In some cases, the weight (mg) ratio of the solubilizing agent to the lipophilic agent is 7:3. In some cases, the weight (mg) ratio of the solubilizing agent to the lipophilic agent is 6:4. In some cases, the weight (mg) ratio of the solubilizing agent to the lipophilic agent is 5:5. In some cases, the weight (mg) ratio of the solubilizing agent to the lipophilic agent is 4:6. In some cases, the weight (mg) ratio of the solubilizing agent to the lipophilic agent is 3:7. In some cases, the weight (mg) ratio of the solubilizing agent to the lipophilic agent is 2:8. In some cases, the weight (mg) ratio of the solubilizing agent to the lipophilic agent is 1:9.

In some embodiments, the range of the solubilizing agent is selected from: about 5% w/w to about 90% w/w, about 10% w/w to about 90% w/w, about 10% w/w to about 80% w/w, about 10% w/w to about 70% w/w, about 10% to about 60% w/w, about 15% w/w to about 90% w/w, about 15% w/w to about 80% w/w, about 15% w/w to about 70% w/w, about 15% w/w to about 60% w/w, about 20% w/w to about 90% w/w, about 20% w/w to about 80% w/w, about 20% w/w to about 70% w/w, about 20% w/w to about 60% w/w, about 22% w/w to about 80% w/w, about 22% w/w to about 68% w/w, about 22% w/w to about 67.5% w/w, about 22% w/w to about 67% w/w, about 22% w/w to about 63% w/w, about 22% w/w to about 60% w/w, about 22% w/w to about 50% w/w, about 22% w/w to about 40% w/w, about 22% w/w to about 36% w/w, about 22% w/w to about 30% w/w, about 22% w/w to about 25% w/w, about 25% w/w to about 80% w/w, about 25% w/w to about 68% w/w, about 25% w/w to about 67.5% w/w, about 25% w/w to about 67% w/w, about 25% w/w to about 63% w/w, about 25% w/w to about 60% w/w, about 25% w/w to about 50% w/w, about 25% w/w to about 40% w/w, about 25% w/w to about 36% w/w, about 25% w/w to about 30% w/w, about 30% w/w to about 80% w/w, about 30% w/w to about 68% w/w, about 30% w/w to about 67.5% w/w, about 30% w/w to about 67% w/w, about 30% w/w to about 63% w/w, about 30% w/w to about 60% w/w, about 30% w/w to about 50% w/w, about 30% w/w to about 40% w/w, about 30% w/w to about 36% w/w, about 10% w/w to about 50% w/w, about 10% w/w to about 40% w/w, about 10% w/w to about 30% w/w, about 15% w/w to about 50% w/w, about 15% w/w to about 40% w/w, about 15% w/w to about 30% w/w, about 20% w/w to about 50% w/w, about 20% w/w to about 40% w/w, about 5% w/w to about 10% w/w, about 5% w/w to about 20% w/w, or about 5% w/w to about 30% w/w. In some instances, the range of the solubilizing agent is from about 25% w/w to about 80% w/w. In some instances, the range of the solubilizing agent is from about 25% w/w to about 68% w/w. In some instances, the range of the solubilizing agent is from about 25% w/w to about 67.5% w/w. In some instances, the range of the solubilizing agent is from about 25% w/w to about 63% w/w. In some instances, the range of the solubilizing agent is from about 25% w/w to about 60% w/w. In some instances, the range of the solubilizing agent is from about 30% w/w to about 80% w/w. In some instances, the range of the solubilizing agent is from about 30% w/w to about 68% w/w. In some instances, the range of the solubilizing agent is from about 30% w/w to about 67.5% w/w. In some instances, the range of the solubilizing agent is from about 30% w/w to about 63% w/w.

In some instances, the composition comprises about 1% w/w, about 2% w/w, about 5% w/w, about 10% w/w, about 15% w/w, about 20% w/w, about 22% w/w, about 25% w/w, about 33% w/w, about 33.8% w/w, about 36% w/w, about 57% w/w, about 57.7% w/w, about 63% w/w, about 67% w/w, about 67.5% w/w, about 67.6% w/w, about 68% w/w, about 68.4% w/w, about 79% w/w, or about 80% w/w of the solubilizing agent. In some cases, the composition comprises about 63% w/w of the solubilizing agent. In some cases, the composition comprises about 67.5% w/w of the solubilizing agent.

In some instances, the solubilizing agent comprises benzyl benzoate. In some embodiments, the range of benzyl benzoate is selected from: about 1% w/w to about 90% w/w, about 2% w/w to about 90% w/w, about 5% w/w to about 90% w/w, about 5% w/w to about 80% w/w, about 5% w/w to about 70% w/w, about 10% w/w to about 90% w/w, about 10% w/w to about 80% w/w, about 10% w/w to about 70% w/w, about 10% to about 60% w/w, about 15% w/w to about 90% w/w, about 15% w/w to about 80% w/w, about 15% w/w to about 70% w/w, about 15% w/w to about 60% w/w, about 20% w/w to about 90% w/w, about 20% w/w to about 80% w/w, about 20% w/w to about 70% w/w, about 20% w/w to about 60% w/w, about 22% w/w to about 80% w/w, about 22% w/w to about 68% w/w, about 22% w/w to about 67.5% w/w, about 22% w/w to about 67% w/w, about 22% w/w to about 63% w/w, about 22% w/w to about 60% w/w, about 22% w/w to about 50% w/w, about 22% w/w to about 40% w/w, about 22% w/w to about 36% w/w, about 22% w/w to about 30% w/w, about 22% w/w to about 25% w/w, about 25% w/w to about 80% w/w, about 25% w/w to about 68% w/w, about 25% w/w to about 67.5% w/w, about 25% w/w to about 67% w/w, about 25% w/w to about 63% w/w, about 25% w/w to about 60% w/w, about 25% w/w to about 50% w/w, about 25% w/w to about 40% w/w, about 25% w/w to about 36% w/w, about 25% w/w to about 300% w/w, about 300% w/w to about 80% w/w, about 30% w/w to about 68% w/w, about 30% w/w to about 67.5% w/w, about 30% w/w to about 67% w/w, about 30% w/w to about 63% w/w, about 30% w/w to about 60% w/w, about 30% w/w to about 50% w/w, about 30% w/w to about 40% w/w, about 30% w/w to about 36% w/w, about 10% w/w to about 50% w/w, about 10% w/w to about 40% w/w, about 10% w/w to about 30% w/w, about 15% w/w to about 50% w/w, about 15% w/w to about 40% w/w, about 15% w/w to about 30% w/w, about 20% w/w to about 50% w/w, about 20% w/w to about 40% w/w, about 5% w/w to about 30% w/w, about 5% w/w to about 20% w/w, about 5% w/w to about 10% w/w, about 2% w/w to about 30% w/w, about 2% w/w to about 20% w/w, about 2% w/w to about 10% w/w, about 1% w/w to about 30% w/w, about 1% w/w to about 20% w/w, about 1% w/w to about 10% w/w, or about 1% w/w to about 5% w/w. In some instances, the range of benzyl benzoate is from about 25% w/w to about 80% w/w. In some instances, the range of benzyl benzoate is from about 25% w/w to about 68% w/w. In some instances, the range of benzyl benzoate is from about 25% w/w to about 67.5% w/w. In some instances, the range of benzyl benzoate is from about 25% w/w to about 63% w/w. In some instances, the range of benzyl benzoate is from about 25% w/w to about 60% w/w. In some instances, the range of benzyl benzoate is from about 30% w/w to about 80% w/w. In some instances, the range of benzyl benzoate is from about 30% w/w to about 68% w/w. In some instances, the range of benzyl benzoate is from about 30% w/w to about 67.5% w/w. In some instances, the range of benzyl benzoate is from about 30% w/w to about 63% w/w. In some instances, the range of benzyl benzoate is from about 10% w/w to about 30% w/w. In some instances, the range of benzyl benzoate is from about 10% w/w to about 20% w/w. In some instances, the range of benzyl benzoate is from about 5% w/w to about 10% w/w. In some instances, the range of benzyl benzoate is from about 2% w/w to about 10% w/w. In some instances, the range of benzyl benzoate is from about 2% w/w to about 5% w/w. In some instances, the range of benzyl benzoate is from about 1% w/w to about 10% w/w. In some instances, the range of benzyl benzoate is from about 1% w/w to about 5% w/w.

In some instances, the composition comprises about 1% w/w, about 2% w/w, about 3% w/w, about 4% w/w, about 5% w/w, about 6% w/w, about 7% w/w, about 8% w/w, about 9% w/w, about 10% w/w, about 15% w/w, about 20% w/w, about 22% w/w, about 25% w/w, about 33% w/w, about 33.8% w/w, about 36% w/w, about 57% w/w, about 57.7% w/w, about 63% w/w, about 67% w/w, about 67.5% w/w, about 67.6% w/w, about 68% w/w, about 68.4% w/w, about 79% w/w, or about 80% w/w of benzyl benzoate. In some cases, the composition comprises about 63% w/w of benzyl benzoate. In some cases, the composition comprises about 67.5% w/w of benzyl benzoate.

In some embodiments, the range of the lipophilic excipient is selected from: about 2.5% w/w to about 8.7% w/w, about 2.5% w/w to about 7.6% w/w, about 2.5% w/w to about 7.4% w/w, about 2.5% w/w to about 7% w/w, about 2.5% w/w to about 6.3% w/w, about 2.5% w/w to about 4% w/w, about 2.5% w/w to about 3.7% w/w, about 2.5% w/w to about 2.8% w/w, about 2.8% w/w to about 9% w/w, about 2.8% w/w to about 8.7% w/w, about 2.8% w/w to about 7.6% w/w, about 2.8% w/w to about 7.4% w/w, about 2.8% w/w to about 7% w/w, about 2.8% w/w to about 6.3% w/w, about 2.8% w/w to about 4% w/w, about 2.8% w/w to about 3.7% w/w, about 3% w/w to about 9% w/w, about 3% w/w to about 8.7% w/w, about 3% w/w to about 7.6% w/w, about 3% w/w to about 7.4% w/w, about 3% w/w to about 7% w/w, about 3% w/w to about 6.3% w/w, about 3% w/w to about 4% w/w, about 6% w/w to about 9% w/w, about 6% w/w to about 8.7% w/w, about 6% w/w to about 7.6% w/w, about 6% w/w to about 7.4% w/w, about 6% w/w to about 7% w/w, about 7% w/w to about 9% w/w, about 7% w/w to about 8.7% w/w, about 7% w/w to about 7.6% w/w, or about 7% w/w to about 7.4% w/w. In some cases, the range of the lipophilic excipient is from about 2.5% w/w to about 7.6% w/w. In some cases, the range of the lipophilic excipient is from about 2.5% w/w to about 7.4% w/w. In some cases, the range of the lipophilic excipient is from about 2.5% w/w to about 7% w/w. In some cases, the range of the lipophilic excipient is from about 2.5% w/w to about 6.3% w/w. In some cases, the range of the lipophilic excipient is from about 2.5% w/w to about 4% w/w. In some cases, the range of the lipophilic excipient is from about 2.5% w/w to about 3.7% w/w. In some cases, the range of the lipophilic excipient is from about 3% w/w to about 7.4% w/w. In some cases, the range of the lipophilic excipient is from about 3% w/w to about 7% w/w. In some cases, the range of the lipophilic excipient is from about 3% w/w to about 6.3% w/w. In some cases, the range of the lipophilic excipient is from about 3% w/w to about 4% w/w.

In some embodiments, the range of the lipophilic excipient is selected from: about 5% w/w to about 90% w/w, about 5% w/w to about 80% w/w, about 5% w/w to about 70% w/w, about 10% w/w to about 90% w/w, about 10% w/w to about 80% w/w, about 10% w/w to about 70% w/w, about 10% to about 60% w/w, about 15% w/w to about 90% w/w, about 15% w/w to about 80% w/w, about 15% w/w to about 70% w/w, about 15% w/w to about 60% w/w, about 20% w/w to about 90% w/w, about 20% w/w to about 80% w/w, about 20% w/w to about 70% w/w, about 20% w/w to about 60% w/w, about 25% w/w to about 90% w/w, about 25% w/w to about 80% w/w, about 25% w/w to about 70% w/w, about 25% w/w to about 60% w/w, about 30% w/w to about 90% w/w, about 30% w/w to about 80% w/w, about 30% w/w to about 70% w/w, about 30% w/w to about 60% w/w, about 40% w/w to about 90% w/w, about 40% w/w to about 80% w/w, about 40% w/w to about 70% w/w, about 50% w/w to about 90% w/w, about 50% w/w to about 80% w/w, about 50% w/w to about 70% w/w, about 60% w/w to about 90% w/w, or about 60% w/w to about 80% w/w. In some cases, the range of the lipophilic excipient is from about 10% w/w to about 90% w/w. In some cases, the range of the lipophilic excipient is from about 20% w/w to about 80% w/w. In some cases, the range of the lipophilic excipient is from about 30% w/w to about 90% w/w. In some cases, the range of the lipophilic excipient is from about 30% w/w to about 60% w/w. In some cases, the range of the lipophilic excipient is from about 50% w/w to about 90% w/w.

In some embodiments, the composition comprises about 2.5% w/w, about 2.8% w/w, about 3.7% w/w, about 4% w/w, about 6.3% w/w, about 7% w/w, about 7.4% w/w, about 7.6% w/w, about 8.7% w/w, or about 9% w/w of the lipophilic excipient. In some cases, the composition comprises about 10% w/w, about 15% w/w, about 20% w/w, about 25% w/w, about 30% w/w, about 35% w/w, about 40% w/w, about 50% w/w, about 60% w/w, about 70% w/w, about 80% w/w, or about 90% w/w of the lipophilic excipient.

In some instances, the lipophilic agent comprises macrogolglycerol ricinolate. In some cases, the range of macrogolglycerol ricinolate is selected from: about 2.5% w/w to about 8.7% w/w, about 2.5% w/w to about 7.6% w/w, about 2.5% w/w to about 7.4% w/w, about 2.5% w/w to about 7% w/w, about 2.5% w/w to about 6.3% w/w, about 2.5% w/w to about 4% w/w, about 2.5% w/w to about 3.7% w/w, about 2.5% w/w to about 2.8% w/w, about 2.8% w/w to about 9% w/w, about 2.8% w/w to about 8.7% w/w, about 2.8% w/w to about 7.6% w/w, about 2.8% w/w to about 7.4% w/w, about 2.8% w/w to about 7% w/w, about 2.8% w/w to about 6.3% w/w, about 2.8% w/w to about 4% w/w, about 2.8% w/w to about 3.7% w/w, about 3% w/w to about 9% w/w, about 3% w/w to about 8.7% w/w, about 3% w/w to about 7.6% w/w, about 3% w/w to about 7.4% w/w, about 3% w/w to about 7% w/w, about 3% w/w to about 6.3% w/w, about 3% w/w to about 4% w/w, about 6% w/w to about 9% w/w, about 6% w/w to about 8.7% w/w, about 6% w/w to about 7.6% w/w, about 6% w/w to about 7.4% w/w, about 6% w/w to about 7% w/w, about 7% w/w to about 9% w/w, about 7% w/w to about 8.7% w/w, about 7% w/w to about 7.6% w/w, or about 7% w/w to about 7.4% w/w. In some cases, the range of macrogolglycerol ricinolate is from about 2.5% w/w to about 7.6% w/w. In some cases, the range of macrogolglycerol ricinolate is from about 2.5% w/w to about 7.4% w/w. In some cases, the range of macrogolglycerol ricinolate is from about 2.5% w/w to about 7% w/w. In some cases, the range of macrogolglycerol ricinolate is from about 2.5% w/w to about 6.3% w/w. In some cases, the range of macrogolglycerol ricinolate is from about 2.5% w/w to about 4% w/w. In some cases, the range of macrogolglycerol ricinolate is from about 2.5% w/w to about 3.7% w/w. In some cases, the range of macrogolglycerol ricinolate is from about 3% w/w to about 7.4% w/w. In some cases, the range of macrogolglycerol ricinolate is from about 3% w/w to about 7% w/w. In some cases, the range of macrogolglycerol ricinolate is from about 3% w/w to about 6.3% w/w. In some cases, the range of macrogolglycerol ricinolate is from about 3% w/w to about 4% w/w.

In some cases, the composition comprises about 2.5% w/w, about 2.8% w/w, about 3.7% w/w, about 4% w/w, about 6.3% w/w, about 7% w/w, about 7.4% w/w, about 7.6% w/w, about 8.7% w/w, or about 9% w/w of macrogolglycerol ricinolate.

In some embodiments, the composition comprises two or more solubilizing agents. For example, the composition may comprise three, four, five, six, or more solubilizing agents. In some instances, the solubilizing agents are selected from benzyl benzoate, diethylene glycol monoethyl ether, propylene glycol monolaurate, glyceryl monocaprylate, propylene glycol monocaprylate, and oleic acid. In some cases, the composition comprises two solubilizing agents selected from benzyl benzoate, diethylene glycol monoethyl ether, propylene glycol monolaurate, glyceryl monocaprylate, propylene glycol monocaprylate, and oleic acid. In some cases, the composition comprises three solubilizing agents selected from benzyl benzoate, diethylene glycol monoethyl ether, propylene glycol monolaurate, glyceryl monocaprylate, propylene glycol monocaprylate, and oleic acid. In some cases, the composition comprises four solubilizing agents selected from benzyl benzoate, diethylene glycol monoethyl ether, propylene glycol monolaurate, glyceryl monocaprylate, propylene glycol monocaprylate, and oleic acid. In some cases, the two or more solubilizing agents have a total w/w percentage of from about 1% w/w to about 90% w/w, about 2% w/w to about 90% w/w, about 5% w/w to about 90% w/w, about 5% w/w to about 80% w/w, about 5% w/w to about 70% w/w, about 10% w/w to about 90% w/w, about 10% w/w to about 80% w/w, about 10% w/w to about 70% w/w, about 10% to about 60% w/w, about 15% w/w to about 90% w/w, about 15% w/w to about 80% w/w, about 15% w/w to about 70% w/w, about 15% w/w to about 60% w/w, about 20% w/w to about 90% w/w, about 20% w/w to about 80% w/w, about 20% w/w to about 70% w/w, about 20% w/w to about 60% w/w, about 30% w/w to about 50% w/w, about 10% w/w to about 20% w/w, about 5% w/w to about 10% w/w, about 1% w/w to about 10% w/w, about 1% w/w to about 5% w/w, about 2% w/w to about 10% w/w, or about 2% w/w to about 5% w/w.

In some embodiments, the composition comprises two or more lipophilic agents. For example, the composition may comprise three, four, five, six, or more lipophilic agents. In some instances, the lipophilic agents are selected from macrogolglycerol ricinolate, polysorbate 80, caprylocaproyl polyoxyl-8 glycerides, glyceryl monooleate, glyceryl monolinoleate, PEG 15 hydroxystearate, PEG 40 hydrogenated castor oil, PEG 35 castor oil, and olive oil. In some cases, the composition comprises two lipophilic agents selected from macrogolglycerol ricinolate, polysorbate 80, caprylocaproyl polyoxyl-8 glycerides, glyceryl monooleate, glyceryl monolinoleate, PEG 15 hydroxystearate, PEG 40 hydrogenated castor oil, PEG 35 castor oil, and olive oil. In some cases, the composition comprises three lipophilic agents selected from macrogolglycerol ricinolate, polysorbate 80, caprylocaproyl polyoxyl-8 glycerides, glyceryl monooleate, glyceryl monolinoleate, PEG 15 hydroxystearate, PEG 40 hydrogenated castor oil, PEG 35 castor oil, and olive oil. In some cases, the composition comprises four lipophilic agents selected from macrogolglycerol ricinolate, polysorbate 80, caprylocaproyl polyoxyl-8 glycerides, glyceryl monooleate, glyceryl monolinoleate, PEG 15 hydroxystearate, PEG 40 hydrogenated castor oil, PEG 35 castor oil, and olive oil. In some cases, the two or more lipophilic agents have a total w/w percentage of from about 1% w/w to about 90% w/w, about 2% w/w to about 90% w/w, about 5% w/w to about 90% w/w, about 5% w/w to about 80% w/w, about 5% w/w to about 70% w/w, about 10% w/w to about 90% w/w, about 10% w/w to about 80% w/w, about 10% w/w to about 70% w/w, about 10% to about 60% w/w, about 15% w/w to about 90% w/w, about 15% w/w to about 80% w/w, about 15% w/w to about 70% w/w, about 15% w/w to about 60% w/w, about 20% w/w to about 90% w/w, about 20% w/w to about 80% w/w, about 20% w/w to about 70% w/w, about 20% w/w to about 60% w/w, about 30% w/w to about 70% w/w, or about 30% w/w to about 50% w/w.

In some embodiments, the composition comprises one or more solubilizing agents and one or more lipophilic agents. The composition can comprise two, three, four, five, or more solubilizing agents and a lipophilic agent. The composition can comprise two solubilizing agents and a lipophilic agent. The composition can comprise three solubilizing agents and a lipophilic agent. The composition can comprise a solubilizing agent and two, three, four, five, or more lipophilic agents. The composition can comprise a solubilizing agent and two lipophilic agents. The composition can comprise a solubilizing agent and three lipophilic agents. The composition can comprise two solubilizing agents and two, three, four, or more lipophilic agents. The solubilizing agents can be selected from benzyl benzoate, diethylene glycol monoethyl ether, propylene glycol monolaurate, glyceryl monocaprylate, propylene glycol monocaprylate, and oleic acid. The lipophilic agents can be selected from macrogolglycerol ricinolate, polysorbate 80, caprylocaproyl polyoxyl-8 glycerides, glyceryl monooleate, glyceryl monolinoleate, PEG 15 hydroxystearate, PEG 40 hydrogenated castor oil, PEG 35 castor oil, and olive oil. A weight (mg) ratio of the solubilizing agent(s) to the lipophilic agent(s) in the composition can be 9:1, 8:2, 7:3, 6:4, 5:5, 4:6, 3:7, 2:8, or 1:9, in which the ratio is the weight of the total solubilizing agent(s) and the weight of the total lipophilic agent(s) in the composition.

In some embodiments, the composition comprises benzyl benzoate, one or more additional solubilizing agents, and one or more lipophilic agents. The composition can comprise benzyl benzoate, one additional solubilizing agent, and one or more lipophilic agents. The composition can comprise benzyl benzoate, two additional solubilizing agents, and one or more lipophilic agents. The composition can comprise benzyl benzoate, three additional solubilizing agents, and one or more lipophilic agents. The composition can comprise benzyl benzoate, four additional solubilizing agents, and one or more lipophilic agents. The composition can comprise benzyl benzoate, one additional solubilizing agent, and one lipophilic agent. The composition can comprise benzyl benzoate, one additional solubilizing agent, and two lipophilic agents. The composition can comprise benzyl benzoate, one additional solubilizing agent, and three lipophilic agents. The composition can comprise benzyl benzoate, one additional solubilizing agent, and four lipophilic agents. The composition can comprise a) benzyl benzoate; b) two, three, four, or more additional solubilizing agent; and c) two, three, four, or more lipophilic agents. The additional solubilizing agent can be selected from diethylene glycol monoethyl ether, propylene glycol monolaurate, glyceryl monocaprylate, propylene glycol monocaprylate, and oleic acid. The lipophilic agents can be selected from macrogolglycerol ricinolate, polysorbate 80, caprylocaproyl polyoxyl-8 glycerides, glyceryl monooleate, glyceryl monolinoleate, PEG 15 hydroxystearate, PEG 40 hydrogenated castor oil, PEG 35 castor oil, and olive oil. A weight (mg) ratio of the solubilizing agent(s) to the lipophilic agent(s) in the composition can be 9:1, 8:2, 7:3, 6:4, 5:5, 4:6, 3:7, 2:8, or 1:9, in which the ratio is the weight of the total solubilizing agent(s) and the weight of the total lipophilic agent(s) in the composition.

In some embodiments, the composition comprises propylene glycol monocaprylate, one or more additional solubilizing agents, and one or more lipophilic agents. The composition can comprise propylene glycol monocaprylate, one additional solubilizing agent, and one or more lipophilic agents. The composition can comprise propylene glycol monocaprylate, two additional solubilizing agents, and one or more lipophilic agents. The composition can comprise propylene glycol monocaprylate, three additional solubilizing agents, and one or more lipophilic agents. The composition can comprise propylene glycol monocaprylate, four additional solubilizing agents, and one or more lipophilic agents. The composition can comprise propylene glycol monocaprylate, one additional solubilizing agent, and one lipophilic agent. The composition can comprise propylene glycol monocaprylate, one additional solubilizing agent, and two lipophilic agents. The composition can comprise propylene glycol monocaprylate, one additional solubilizing agent, and three lipophilic agents. The composition can comprise propylene glycol monocaprylate, one additional solubilizing agent, and four lipophilic agents. The composition can comprise a) propylene glycol monocaprylate; b) two, three, four, or more additional solubilizing agent; and c) two, three, four, or more lipophilic agents. The additional solubilizing agent can be selected from benzyl benzoate, diethylene glycol monoethyl ether, propylene glycol monolaurate, glyceryl monocaprylate, and oleic acid. The lipophilic agents can be selected from macrogolglycerol ricinolate, polysorbate 80, caprylocaproyl polyoxyl-8 glycerides, glyceryl monooleate, glyceryl monolinoleate, PEG 15 hydroxystearate, PEG 40 hydrogenated castor oil, PEG 35 castor oil, and olive oil. A weight (mg) ratio of the solubilizing agent(s) to the lipophilic agent(s) in the composition can be 9:1, 8:2, 7:3, 6:4, 5:5, 4:6, 3:7, 2:8, or 1:9, in which the ratio is the weight of the total solubilizing agent(s) and the weight of the total lipophilic agent(s) in the composition.

In some embodiments, the composition comprises glyceryl monocaprylate, one or more additional solubilizing agents, and one or more lipophilic agents. The composition can comprise glyceryl monocaprylate, one additional solubilizing agent, and one or more lipophilic agents. The composition can comprise glyceryl monocaprylate, two additional solubilizing agents, and one or more lipophilic agents. The composition can comprise glyceryl monocaprylate, three additional solubilizing agents, and one or more lipophilic agents. The composition can comprise glyceryl monocaprylate, four additional solubilizing agents, and one or more lipophilic agents. The composition can comprise glyceryl monocaprylate, one additional solubilizing agent, and one lipophilic agent. The composition can comprise glyceryl monocaprylate, one additional solubilizing agent, and two lipophilic agents. The composition can comprise glyceryl monocaprylate, one additional solubilizing agent, and three lipophilic agents. The composition can comprise glyceryl monocaprylate, one additional solubilizing agent, and four lipophilic agents. The composition can comprise a) glyceryl monocaprylate; b) two, three, four, or more additional solubilizing agent; and c) two, three, four, or more lipophilic agents. The additional solubilizing agent can be selected from benzyl benzoate, diethylene glycol monoethyl ether, propylene glycol monolaurate, propylene glycol monocaprylate, and oleic acid. The lipophilic agents can be selected from macrogolglycerol ricinolate, polysorbate 80, caprylocaproyl polyoxyl-8 glycerides, glyceryl monooleate, glyceryl monolinoleate, PEG 15 hydroxystearate, PEG 40 hydrogenated castor oil, PEG 35 castor oil, and olive oil. A weight (mg) ratio of the solubilizing agent(s) to the lipophilic agent(s) in the composition can be 9:1, 8:2, 7:3, 6:4, 5:5, 4:6, 3:7, 2:8, or 1:9, in which the ratio is the weight of the total solubilizing agent(s) and the weight of the total lipophilic agent(s) in the composition.

In some embodiments, the composition comprises PEG 35 castor oil, one or more additional lipophilic agents, and one or more solubilizing agents. The composition can comprise PEG 35 castor oil, one additional lipophilic agent, and one or more solubilizing agents. The composition can comprise PEG 35 castor oil, two additional lipophilic agents, and one or more solubilizing agents. The composition can comprise PEG 35 castor oil, three additional lipophilic agents, and one or more solubilizing agents. The composition can comprise PEG 35 castor oil, four additional lipophilic agents, and one or more solubilizing agents. The composition can comprise PEG 35 castor oil, one additional lipophilic agent, and two solubilizing agents. The composition can comprise PEG 35 castor oil, one additional lipophilic agent, and three solubilizing agents. The composition can comprise PEG 35 castor oil, one additional lipophilic agent, and four solubilizing agents. The composition can comprise a) PEG 35 castor oil; b) two, three, four, or more additional lipophilic agents; and c) two, three, four, or more solubilizing agents. The additional lipophilic agents can be selected from macrogolglycerol ricinolate, polysorbate 80, caprylocaproyl polyoxyl-8 glycerides, glyceryl monooleate, glyceryl monolinoleate, PEG 15 hydroxystearate, PEG 40 hydrogenated castor oil, and olive oil. The additional solubilizing agent can be selected from benzyl benzoate, diethylene glycol monoethyl ether, propylene glycol monolaurate, glyceryl monocaprylate, propylene glycol monocaprylate, or oleic acid. A weight (mg) ratio of the solubilizing agent(s) to the lipophilic agent(s) in the composition can be 9:1, 8:2, 7:3, 6:4, 5:5, 4:6, 3:7, 2:8, or 1:9, in which the ratio is the weight of the total solubilizing agent(s) and the weight of the total lipophilic agent(s) in the composition.

In some embodiments, the composition comprises benzyl benzoate, propylene glycol monocaprylate, and one or more lipophilic agents. In some instances, the composition comprises benzyl benzoate, propylene glycol monocaprylate, and one lipophilic agent. In some instances, the composition comprises benzyl benzoate, propylene glycol monocaprylate, and two or more lipophilic agents. The lipophilic agents can be selected from macrogolglycerol ricinolate, polysorbate 80, caprylocaproyl polyoxyl-8 glycerides, glyceryl monooleate, glyceryl monolinoleate, PEG 15 hydroxystearate, PEG 40 hydrogenated castor oil, PEG 35 castor oil, and olive oil. The lipophilic agent can be PEG 35 castor oil. The composition can comprise benzyl benzoate, propylene glycol monocaprylate, and PEG 35 castor oil. A weight (mg) ratio of the solubilizing agent(s) to the lipophilic agent(s) in the composition can be 9:1, 8:2, 7:3, 6:4, 5:5, 4:6, 3:7, 2:8, or 1:9, in which the ratio is the weight of the total solubilizing agent(s) and the weight of the total lipophilic agent(s) in the composition.

In some embodiments, the composition comprises propylene glycol monocaprylate and one or more lipophilic agents. The lipophilic agents can be selected from macrogolglycerol ricinolate, polysorbate 80, caprylocaproyl polyoxyl-8 glycerides, glyceryl monooleate, glyceryl monolinoleate, PEG 15 hydroxystearate, PEG 40 hydrogenated castor oil, PEG 35 castor oil, and olive oil. The lipophilic agent can be PEG 35 castor oil. The composition can comprise propylene glycol monocaprylate and PEG 35 castor oil. A weight (mg) ratio of the solubilizing agent(s) to the lipophilic agent(s) in the composition can be 9:1, 8:2, 7:3, 6:4, 5:5, 4:6, 3:7, 2:8, or 1:9, in which the ratio is the weight of the total solubilizing agent(s) and the weight of the total lipophilic agent(s) in the composition.

In some embodiments, the composition comprises glyceryl monocaprylate and one or more lipophilic agents. The lipophilic agents can be selected from macrogolglycerol ricinolate, polysorbate 80, caprylocaproyl polyoxyl-8 glycerides, glyceryl monooleate, glyceryl monolinoleate, PEG 15 hydroxystearate, PEG 40 hydrogenated castor oil, PEG 35 castor oil, and olive oil. The lipophilic agent can be PEG 35 castor oil. The composition can comprise glyceryl monocaprylate and PEG 35 castor oil. A weight (mg) ratio of the solubilizing agent(s) to the lipophilic agent(s) in the composition can be 9:1, 8:2, 7:3, 6:4, 5:5, 4:6, 3:7, 2:8, or 1:9, in which the ratio is the weight of the total solubilizing agent(s) and the weight of the total lipophilic agent(s) in the composition.

In some embodiments, the composition further comprises one or more additional excipients. In some instances, the one or more additional excipients comprise a flavoring excipient, a preservative, or a diluent. In some instances, the composition comprises about 50% w/w of the total amount of the one or more additional excipients.

In some embodiments in the presence of the one or more excipients, the range of the 17-HPC is selected from: about 3% w/w to about 36% w/w, about 3% w/w to about 25% w/w, about 3% w/w to about 24% w/w, about 3% w/w to about 12% w/w, about 6% w/w to about 36% w/w, about 6% w/w to about 25% w/w, about 6% w/w to about 24% w/w, about 6% w/w to about 12% w/w, about 12% w/w to about 36% w/w, about 12% w/w to about 25% w/w, about 12% w/w to about 24% w/w, about 24% w/w to about 36% w/w, or about 25% w/w to about 36% w/w.

In some cases, in the presence of the one or more excipients, the composition comprises about 3% w/w, 6% w/w, 12% w/w, 24% w/w, 25% w/w, or 36% w/w of 17-HPC.

In some instances, in the presence of the one or more excipients, the range of the 2-component solvent system is selected from: about 14% w/w to about 57% w/w, about 14% w/w to about 44% w/w, about 14% w/w to about 38% w/w, about 14% w/w to about 26% w/w, about 14% w/w to about 25% w/w, about 25% w/w to about 57% w/w, about 25% w/w to about 44% w/w, about 25% w/w to about 38% w/w, about 26% w/w to about 57% w/w, about 26% w/w to about 44% w/w, about 26% w/w to about 38% w/w, or about 38% w/w to about 57% w/w.

In some embodiments, the amount of 17-PC and the 2-component solvent system in the composition is illustrated in Table 1A, Table 1B, Table 1C, and Table 1D. In some instances, the amount of 17-HPC and the 2-component solvent system in the composition is illustrated in Table 2A and Table 2B.

TABLE 1A
Batch proportions per 1000 mg Formulation
by weight-Without Flavoring or Preservative

| Compounds | Amount (mg) | % w/w | Amount (mg) | % w/w | Amount (mg) | % w/w |
|---|---|---|---|---|---|---|
| 17-HPC | 120 | 12 | 240 | 24 | 250 | 25 |
| 2-component | 880 | 88 | 760 | 76 | 750 | 75 |
| Solvent | | | | | | |
| System | | | | | | |

TABLE 1B
Batch proportions per 1000 mg Formulation by
weight-Without Flavoring or Preservatives

| Compounds | Amount (mg) | % w/w | Amount (mg) | % w/w | Amount (mg) | % w/w |
|---|---|---|---|---|---|---|
| 17-HPC | 360 | 36 | 740 | 74 | 750 | 75 |
| 2-component | 640 | 64 | 260 | 26 | 250 | 25 |
| Solvent | | | | | | |
| System | | | | | | |

TABLE 1C
Batch proportions per 1000 mg Formulation by weight-
With Flavoring, Preservatives, or other diluting agents

| Compounds | Amount (mg) | % w/w | Amount (mg) | % w/w | Amount (mg) | % w/w |
|---|---|---|---|---|---|---|
| 17-HPC | 360 | 36 | 250 | 25 | 240 | 24 |
| 2-component | 140 | 14 | 250 | 25 | 260 | 26 |
| Solvent | | | | | | |
| System | | | | | | |
| Flavoring, | 500 | 50 | 500 | 50 | 500 | 50 |
| Preservatives, | | | | | | |
| or Other | | | | | | |
| Diluents | | | | | | |

TABLE 1D
Batch proportions per 1000 mg Formulation by weight-
With Flavoring, Preservatives, or other diluting agents

| Compounds | Amount (mg) | % w/w | Amount (mg) | % w/w | Amount (mg) | % w/w |
|---|---|---|---|---|---|---|
| 17-HPC | 120 | 12 | 60 | 6 | 30 | 3 |
| 2-component | 380 | 38 | 440 | 44 | 570 | 57 |
| Solvent | | | | | | |
| System | | | | | | |
| Flavoring, | 500 | 50 | 500 | 50 | 500 | 50 |
| Preservatives, | | | | | | |
| or Other | | | | | | |
| Diluents | | | | | | |

TABLE 2A
Batch proportions per 1000 mL Formulation by
Volume-Without Flavoring or Preservatives

| Compounds | Amount | | % w/v | Amount | | % w/v | Amount | | % w/v |
|---|---|---|---|---|---|---|---|---|---|
| 17-HPC | 120 | mg | 12 | 240 | mg | 24 | 360 | mg | 36 |
| 2-component | | | | | | | | | |
| Solvent System | | | | | | | | | |
| QS to final | 1000 | mL | QS ad | 1000 | mL | QS ad | 1000 | mL | QS ad |

volume:

TABLE 2B
Batch proportions per 1000 mL Formulation by Volume-
With Flavoring, Preservatives, or other diluting agents

| Compounds | Amount | | % w/v | Amount | | % w/v | Amount | | % w/v |
|---|---|---|---|---|---|---|---|---|---|
| 17-HPC | 120 | mg | 6 | 360 | mg | 18 | 720 | mg | 36 |
| 2-component | | | | | | | | | |
| Solvent System | | | | | | | | | |
| QS to | 500 | mL | | 500 | mL | | 500 | mL | |
| intermediate | | | | | | | | | |
| volume | | | | | | | | | |
| Flavoring, Preservatives, | | | | | | | | | |
| or Other Diluents | | | | | | | | | |
| QS to final | 1000 | mL | | 1000 | mL | | 1000 | mL | |

volume

In some instances, the composition comprises about 24% w/w of 17-HPC and about 76% w/w of the 2-component solvent system comprising a solubilizing agent and a lipophilic excipient.

In some instances, the composition comprises about 24% w/w of 17-HPC and about 76% w/w of the 2-component solvent system comprising benzyl benzoate and macrogolglycerol ricinolate.

In some instances, the composition comprises about 25% w/w of 17-HPC and about 75% w/w of the 2-component solvent system comprising a solubilizing agent and a lipophilic excipient.

In some instances, the composition comprises about 25% w/w of 17-HPC and about 75% w/w of the 2-component solvent system comprising benzyl benzoate and macrogolglycerol ricinolate.

In some instances, the composition comprises about 36% w/w of 17-HPC and about 64% w/w of the 2-component solvent system comprising a solubilizing agent and a lipophilic excipient.

In some instances, the composition comprises about 36% w/w of 17-HPC and about 64% w/w of the 2-component solvent system comprising benzyl benzoate and macrogolglycerol ricinolate.

In some instances, the composition is a solution.

In some instances, the composition is formulated for oral administration.

In some instances, the composition is formulated as an oral capsule, optionally a soft gelatin capsule.

In some instances, the composition is formulated as an injection. In some cases, the compositions of Table 1A, Table 1B, Table 1C, and Table 1D are each independently formulated as an injection.

In some cases, the compositions of Table 2A and Table 2B are formulated as an injection.

In certain embodiments, disclosed herein is a solution comprising 17-HPC. In some embodiments, the solution comprises, consisting essentially or, or consisting of a range of from about 120 mg/mL to about 360 mg/mL of 17-alpha hydroxyprogesterone caproate (17-HPC), one or more solubilizing agents, and one or more lipophilic agents. In some embodiments, the solution comprises, consisting essentially or, or consisting of a range of from about 120 mg/mL to about 360 mg/mL of 17-alpha hydroxyprogesterone caproate (17-HPC) and a 2-component solvent system.

In some instances, the range of 17-HPC is selected from: about 120 mg/mL to about 240 mg/mL, or about 240 mg/mL to about 360 mg/mL.

In some instances, the solution comprises about 120 mg/mL, about 240 mg/mL, or about 360 mg/mL of 17-HPC.

In some embodiments, the 2-component solvent system comprises a solubilizing agent and a lipophilic excipient, optionally a range of the solubilizing agent and a range of the lipophilic excipient. In some instances, the solubilizing agent comprises benzyl benzoate, diethylene glycol monoethyl ether, propylene glycol monolaurate, glyceryl monocaprylate, propylene glycol monocaprylate, or oleic acid. In some instances, the solubilizing agent comprises benzyl benzoate, diethylene glycol monoethyl ether, propylene glycol monolaurate, glyceryl monocaprylate, or oleic acid. In some instances, the solubilizing agent comprises benzyl benzoate. In some instances, the lipophilic excipient comprises macrogolglycerol ricinolate, polysorbate 80, caprylocaproyl polyoxyl-8 glycerides, glyceryl monooleate, glyceryl monolinoleate, PEG 15 hydroxystearate, PEG 40 hydrogenated castor oil, PEG 35 castor oil, or olive oil. In some instances, the lipophilic excipient comprises macrogolglycerol ricinolate, polysorbate 80, or caprylocaproyl polyoxyl-8 glycerides. In some instances, the lipophilic excipient comprises macrogolglycerol ricinolate.

In some embodiments, the composition comprises 17-HPC, the 2-component solvent system, and one or more additional progestins. In some instances, the one or more additional progestins comprise a hydroxyprogesterone ester. In some cases, the composition comprises 17-HPC, one or more additional hydroxyprogesterone esters (e.g., one, two, or three additional hydroxyprogesterone esters), and the 2-component solvent system. In some instances, the 2-component solvent system comprises a solubilizing agent and a lipophilic excipient, optionally a range of the solubilizing agent and a range of the lipophilic excipient. In some instances, the solubilizing agent comprises benzyl benzoate, diethylene glycol monoethyl ether, propylene glycol monolaurate, glyceryl monocaprylate, propylene glycol monocaprylate, or oleic acid. In some instances, the solubilizing agent comprises benzyl benzoate, diethylene glycol monoethyl ether, propylene glycol monolaurate, glyceryl monocaprylate, or oleic acid. In some instances, the solubilizing agent comprises benzyl benzoate. In some instances, the lipophilic excipient comprises macrogolglycerol ricinolate, polysorbate 80, caprylocaproyl polyoxyl-8 glycerides, glyceryl monooleate, glyceryl monolinoleate, PEG 15 hydroxystearate, PEG 40 hydrogenated castor oil, PEG 35 castor oil, or olive oil. In some instances, the lipophilic excipient comprises macrogolglycerol ricinolate, polysorbate 80, or caprylocaproyl polyoxyl-8 glycerides. In some instances, the lipophilic excipient comprises macrogolglycerol ricinolate.

In some embodiments, the composition comprises 17-HPC, one or more solubilizing agents, one or more lipophilic agents, and one or more additional progestins. In some instances, the one or more additional progestins comprise a hydroxyprogesterone ester. In some cases, the composition comprises 17-HPC, one or more additional hydroxyprogesterone esters (e.g., one, two, or three additional hydroxyprogesterone esters), one or more solubilizing agents, and one or more lipophilic agents. In some instances, the one or more solubilizing agents (e.g., two, three, four, or more solubilizing agents) are selected from benzyl benzoate, diethylene glycol monoethyl ether, propylene glycol monolaurate, glyceryl monocaprylate, propylene glycol monocaprylate, and oleic acid. In some instances, the one or more lipophilic agents (e.g., two, three, four, or more lipophilic agents) are selected from macrogolglycerol ricinolate, polysorbate 80, caprylocaproyl polyoxyl-8 glycerides, glyceryl monooleate, glyceryl monolinoleate, PEG 15 hydroxystearate, PEG 40 hydrogenated castor oil, PEG 35 castor oil, and olive oil.

In some embodiments, the one or more additional progestins comprise hydroxyprogesterone acetate or hydroxyprogesterone heptanoate. In some instances, the composition comprises 17-HPC, one or more additional progestins selected from hydroxyprogesterone acetate and hydroxyprogesterone heptanoate, and the 2-component solvent system. In some cases, the composition comprises 17-HPC, hydroxyprogesterone acetate, and the 2-component solvent system. In some cases, the composition comprises 17-HPC, hydroxyprogesterone heptanoate, and the 2-component solvent system. In some cases, the composition comprises 17-HPC, hydroxyprogesterone acetate, hydroxyprogesterone heptanoate, and the 2-component solvent system. In some instances, the 2-component solvent system comprises a solubilizing agent and a lipophilic excipient, optionally a range of the solubilizing agent and a range of the lipophilic excipient. In some instances, the solubilizing agent comprises benzyl benzoate, diethylene glycol monoethyl ether, propylene glycol monolaurate, glyceryl monocaprylate, propylene glycol monocaprylate, or oleic acid. In some instances, the solubilizing agent comprises benzyl benzoate, diethylene glycol monoethyl ether, propylene glycol monolaurate, glyceryl monocaprylate, or oleic acid. In some instances, the solubilizing agent comprises benzyl benzoate. In some instances, the lipophilic excipient comprises macrogolglycerol ricinolate, polysorbate 80, caprylocaproyl polyoxyl-8 glycerides, glyceryl monooleate, glyceryl monolinoleate, PEG 15 hydroxystearate, PEG 40 hydrogenated castor oil, PEG 35 castor oil, or olive oil. In some instances, the lipophilic excipient comprises macrogolglycerol ricinolate, polysorbate 80, or caprylocaproyl polyoxyl-8 glycerides. In some instances, the lipophilic excipient comprises macrogolglycerol ricinolate.

In some embodiments, the one or more additional progestins comprise hydroxyprogesterone acetate or hydroxyprogesterone heptanoate. In some instances, the composition comprises 17-HPC, one or more additional progestins selected from hydroxyprogesterone acetate and hydroxyprogesterone heptanoate, one or more solubilizing agents, and one or more lipophilic agents. In some cases, the composition comprises 17-HPC, hydroxyprogesterone acetate, one or more solubilizing agents, and one or more lipophilic agents. In some cases, the composition comprises 17-HPC, hydroxyprogesterone heptanoate, one or more solubilizing agents, and one or more lipophilic agents. In some cases, the composition comprises 17-HPC, hydroxyprogesterone acetate, hydroxyprogesterone heptanoate, one or more solubilizing agents, and one or more lipophilic agents. In some instances, the one or more solubilizing agents (e.g., two, three, four, or more solubilizing agents) are selected from benzyl benzoate, diethylene glycol monoethyl ether, propylene glycol monolaurate, glyceryl monocaprylate, propylene glycol monocaprylate, and oleic acid. In some instances, the one or more lipophilic agents (e.g., two, three, four, or more lipophilic agents) are selected from macrogolglycerol ricinolate, polysorbate 80, caprylocaproyl polyoxyl-8 glycerides, glyceryl monooleate, glyceryl monolinoleate, PEG 15 hydroxystearate, PEG 40 hydrogenated castor oil, PEG 35 castor oil, and olive oil.

In some embodiments, the range of the lipophilic excipient is selected from: about 28 mg/mL to about 76 mg/mL, about 28 mg/mL to about 76 mg/mL, about 38 mg/mL to about 74 mg/mL, or about 74 mg/mL to about 87 mg/mL. In some cases, the lipophilic excipient comprises macrogolglycerol ricinolate, polysorbate 80, or caprylocaproyl polyoxyl-8 glycerides.

In some instances, the solution comprises about 25 mg/mL, about 28 mg/mL, about 63 mg/mL, about 74 mg/mL, about 76 mg/mL, or about 87 mg/mL of the lipophilic excipient. In some cases, the lipophilic excipient comprises macrogolglycerol ricinolate, polysorbate 80, or caprylocaproyl polyoxyl-8 glycerides.

In some cases, the lipophilic excipient comprises macrogolglycerol ricinolate. In some cases, the range of macrogolglycerol ricinolate is selected from: about 28 mg/mL to about 76 mg/mL, about 28 mg/mL to about 76 mg/mL, about 38 mg/mL to about 74 mg/mL, or about 74 mg/mL to about 87 mg/mL.

In some cases, the solution comprises about 25 mg/mL, about 28 mg/mL, about 63 mg/mL, about 74 mg/mL, about 76 mg/mL, or about 87 mg/mL of macrogolglycerol ricinolate.

In some embodiments, the range of the solubilizing agent is selected from: about 225 mg/mL to about 793 mg/mL, about 252 mg/mL to about 684 mg/mL, about 252 mg/mL to about 676 mg/mL, about 577 mg/mL to about 793 mg/mL, or about 577 mg/mL to about 684 mg/mL. In some cases, the solubilizing agent comprises benzyl benzoate, diethylene glycol monoethyl ether, propylene glycol monolaurate, glyceryl monocaprylate, or oleic acid.

In some instances, the solution comprises about 225 mg/mL, about 252 mg/mL, about 577 mg/mL, about 676 mg/mL, about 684 mg/mL, or about 793 mg/mL of the solubilizing agent. In some cases, the solubilizing agent comprises benzyl benzoate, diethylene glycol monoethyl ether, propylene glycol monolaurate, glyceryl monocaprylate, or oleic acid.

In some cases, the solubilizing agent comprises benzyl benzoate. In some cases, the range of benzyl benzoate is selected from: about 225 mg/mL to about 793 mg/mL, about 252 mg/mL to about 684 mg/mL, about 252 mg/mL to about 676 mg/mL, about 577 mg/mL to about 793 mg/mL, or about 577 mg/mL to about 684 mg/mL.

In some cases, the solution comprises about 225 mg/mL, about 252 mg/mL, about 577 mg/mL, about 676 mg/mL, about 684 mg/mL, or about 793 mg/mL of benzyl benzoate.

In some instances, the solution is formulated for oral administration.

In some instances, the solution is formulated as an injection.

In certain embodiments, disclosed herein is a soft gelatin capsule comprising a liquid filing comprising 17-alpha hydroxyprogesterone caproate (17-HPC), one or more solubilizing agents, and one or more lipophilic agents; and a capsule shell encapsulating the liquid filling. In certain embodiments, disclosed herein is a soft gelatin capsule comprising a liquid filing comprising 17-alpha hydroxyprogesterone caproate (17-HPC) and a 2-component solvent system; and a capsule shell encapsulating the liquid filling. In some embodiments, the liquid filling comprises a range of 17-HPC, optionally selected from: about 12% w/w to about 36% w/w, about 12% w/w to about 25% w/w, about 12% w/w to about 24% w/w, about 24% w/w to about 36% w/w, or about 25% w/w to about 36% w/w.

In some embodiments, also disclosed herein is a soft gelatin capsule comprising a liquid filing comprising 17-alpha hydroxyprogesterone caproate (17-HPC), one or more solubilizing agents (e.g., two, three, four, or more solubilizing agents), one or more lipophilic agents (e.g., two, three, four, or more lipophilic agents), and a capsule shell encapsulating the liquid filling. In some embodiments, the liquid filling comprises a range of 17-HPC, optionally selected from: about 12% w/w to about 36% w/w, about 12% w/w to about 25% w/w, about 12% w/w to about 24% w/w, about 24% w/w to about 36% w/w, or about 25% w/w to about 36% w/w.

In some cases, the liquid filling comprises about 12% w/w, about 24% w/w, 25% w/w, about 30% w/w/, or about 36% w/w of 17-HPC. In some cases, the liquid filling comprises about 24 wt % of 17-HPC. In some cases, the liquid filling comprises about 36 wt % of 17-HPC.

In some embodiments, the liquid filling comprises a range of the 2-component solvent system. In some instances, the range of the 2-component solvent system is selected from: about 64% w/w to about 88% w/w, about 64% w/w to about 76% w/w, about 64% w/w to about 75% w/w, or about 75% w/w to about 88% w/w. In some instances, the liquid filling comprises about 64% w/w, about 75% w/w, about 76% w/w, or about 88% w/w of the 2-component solvent system.

In some instances, the 2-component solvent system comprises a solubilizing agent and a lipophilic excipient. In some instances, the solubilizing agent comprises benzyl benzoate, diethylene glycol monoethyl ether, propylene glycol monolaurate, glyceryl monocaprylate, or oleic acid. In some instances, the solubilizing agent comprises benzyl benzoate. In some instances, the lipophilic excipient comprises macrogolglycerol ricinolate, polysorbate 80, or caprylocaproyl polyoxyl-8 glycerides. In some instances, the lipophilic excipient comprises macrogolglycerol ricinolate.

In some cases, the soft gelatin capsule is formulated as an immediate release gel cap. As used herein, the term “immediate release” in the context of the gel cap or gel capsule refers to a rapid release of 17-HPC from the capsule over a shortened period of time. The shortened period of time comprises over 1 minute, 2 minutes, 3 minutes, 4 minutes, 5 minutes, 6 minutes, 7 minutes, 8 minutes, 9 minutes, 10 minutes, 15 minutes, 20 minutes, or 30 minutes. In some cases, the shortened period of time comprises at most 1 minute, 2 minutes, 3 minutes, 4 minutes, 5 minutes, 6 minutes, 7 minutes, 8 minutes, 9 minutes, 10 minutes, 15 minutes, 20 minutes, or 30 minutes.

In some cases, the soft gelatin capsule is formulated as a modified release. In some instances, the term modified release refers to a drug release (e.g., 17-HPC release) that occurs after a defined time post administration, or for a prolonged period of time, or to a specific target in the body. In some instances, the modified release comprises a delayed release, an extended release, or a controlled release. As used here, the term delayed release comprises a release of 17-HPC that is delayed by about 10 minutes, 15 minutes, 20 minutes, 30 minutes, 1 hour, 2 hours, 3 hours, 4 hours, 5 hours, 6 hours, 8 hours, 10 hours, 12 hours, or more. In some instances, the release of the 17-HPC is delayed until the drug is passed into the small intestine of a subject.

In some cases, the soft gelatin capsule is formulated as an extended release. In some cases, the extended release comprises a prolonged release, e.g., over the course of 20 minutes, 30 minutes, 1 hour, 2 hours, 6 hours, 12 hours, or more, to reduce dosing frequency.

In some cases, the soft gelatin capsule is formulated as a controlled release. As used herein, the term “controlled release” refers to the concentration of the drug (e.g., 17-HPC) released each time is the same.

In some cases, the soft gelatin capsule is formulated as a sustained release. As used herein, the term “sustained release” refers to the release of the drug (e.g., 17-HPC) at a predetermined rate to maintain a constant drug concentration for a specific period of time. In some instances, the sustained release rate is achieved to minimize side effects.

In some instances, the capsule shell comprises gelatin and non-gelatin materials. Exemplary non-gelatin materials include, but are not limited to, plasticizers such as glycerin or sorbitol, coloring agents, preservatives, disintegrants, or lubricants.

In some embodiments, a composition described herein comprises a range of 17-HPC, a range of benzyl benzoate, and a range of a surfactant comprising a hydrophilic-lipophilic balance (HLB) value of from about 12 to about 15. In some instances, the surfactant comprises caprylocaproyl polyoxyl-8 glycerides (HLB value: about 12), macrogolgylcerol ricinoleate (HLB value: about 12-14), or polysorbate 80 (HLB value: about 15). In some instances, the range of benzyl benzoate is from about 10% w/w to about 90% w/w. In some cases, the range of the surfactant is from about 10% w/w to about 90% w/w. In some cases, the composition comprises a formulation as illustrated in Table 3.

TABLE 3

| | 17-HPC | Benzyl benzoate | | |
|---|---|---|---|---|
| Solution | (mg/g) | (wt. %) | Surfactant | Wt. % |
| A | 150 | 60 | Caprylocaproyl polyoxyl-8 glycerides | 40 |
| B | 75 | 30 | Caprylocaproyl polyoxy1-8 glycerides | 70 |
| C | 37.5 | 10 | Caprylocaproyl polyoxy1-8 glycerides | 90 |
| D | 300 | 90 | Macrogolglycerol ricinoleate | 10 |
| E | 150 | 50 | Macrogolglycerol ricinoleate | 50 |
| F | 300 | 90 | Polysorbate 80 | 10 |
| G | 150 | 50 | Polysorbate 80 | 50 |

In some embodiments, a composition described herein comprises a range of 17-HPC and a lipophilic excipient selected from castor oil (CAS 8001-79-4) and peanut oil. In some instances, the composition comprises about 30 wt % of 17-HPC and about 70 wt % of castor oil or peanut oil. In some cases, 17-HPC in the composition is micronized. In some cases, the Dv10, Dv50, and Dv90 values of the micronized 17-HPC are about 1.27 μm, 5.55 μm, and 14 μm.

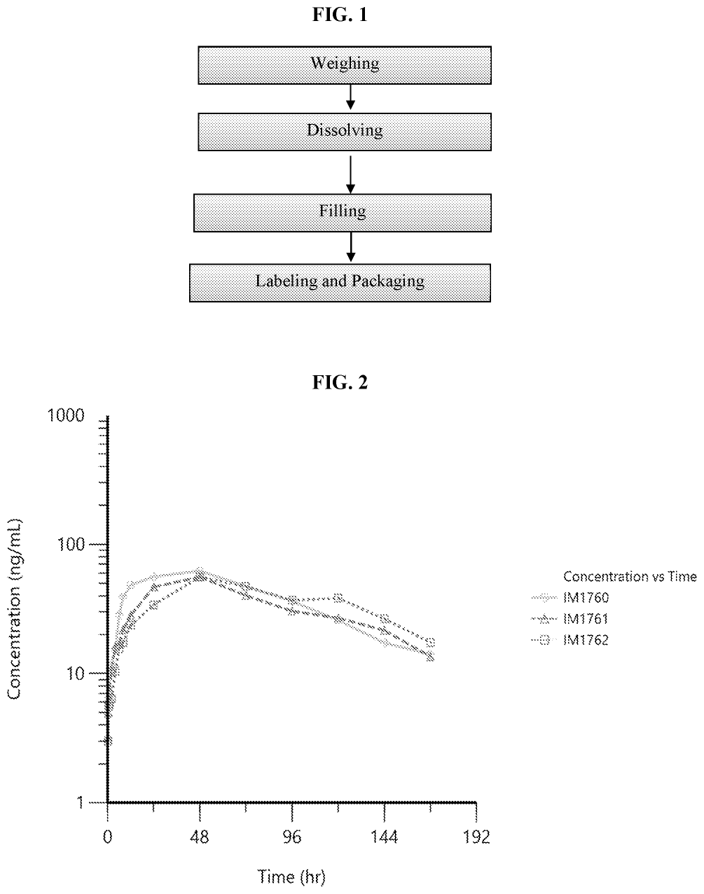

In some embodiments, the composition, the solution, or the soft gelatin capsule is prepared by a manufacturing process illustrated in FIG. 1. As illustrated in FIG. 1, the method comprises weighing the active pharmaceutical ingredient (API) 17-HPC, the solubilizing agent, and the lipophilic excipient prior to dissolving the API in the solubilizing agent and the lipophilic excipient. The solution is subsequently filled in a respective carrier, e.g., into a capsule for soft gelatin capsule, or a container or vial.

In some embodiments, the composition, the solution, or the soft gelatin capsule is assessed to ensure that the composition meets quality specifications. In some instances, the quality specifications are specifications from the U.S. Food & Drug Administration (FDA). In some instances, the quality specification are specifications from a foreign Food & Drug Administration that is a counterpart to the US FDA. In some cases, the quality specification comprises one or more of appearance assessment, identification, impurity assessment, uniformity of dosage, residual solvent, and microbial limit. In some cases, the quality specification comprises one or more of appearance assessment, identification, impurity assessment, and microbial limit.

In some embodiments, the identification and the impurity assessment of the composition, the solution, or the liquid filing of the soft gelatin capsule are carried out by a high-performance liquid chromatography (HPLC) method. Exemplary IPLC methods include normal-phase, reverse-phase, size-exclusion, or ion-exchange chromatography methods.

In some embodiments, the composition is stable for about 1, 3, 6, 9, 12, 18, 24, 26, or more months. As utilized herein, the term “stable” refers to less than about 10%, 9%, 8%, 7%, 6%, 5%, 4%, 3%, 2%, or 1% of an impurity. In some instances, the composition is stable for about 1, 3, 6, 9, 12, 18, 24, 26, or more months at a specified storage condition. In some cases, the storage condition is from about 20° C. to about 28° C., from about 22° C. to about 26° C., or from about 23° C. to about 27° C. In some cases, the storage condition comprises about 55%, about 60%, about 65%, about 70%, about 75%, or about 80% humidity.

In some instances, the solution is stable for about 1, 3, 6, 9, 12, 18, 24, 26, or more months. In some instances, the solution is stable for about 1, 3, 6, 9, 12, 18, 24, 26, or more months at a specified storage condition. In some cases, the storage condition is from about 20° C. to about 28° C., from about 22° C. to about 26° C., or from about 23° C. to about 27° C. In some cases, the storage condition comprises about 55%, about 60%, about 65%, about 70%, about 75%, or about 80% humidity.

In some embodiments, the liquid filling of the soft gelatin capsule is stable for about 1, 3, 6, 9, 12, 18, 24, 26, or more months. In some instances, the liquid filling of the soft gelatin capsule is stable for about 1, 3, 6, 9, 12, 18, 24, 26, or more months at a specified storage condition. In some cases, the storage condition is from about 20° C. to about 28° C., from about 22° C. to about 26° C., or from about 23° C. to about 27° C. In some cases, the storage condition comprises about 55%, about 60%, about 65%, about 70%, about 75%, or about 80% humidity.

In some embodiments, the composition has less than about 10%, 9%, 8%, 7%, 6%, 5%, 4%, 3%, 2%, or 1% degradation at a storage condition. In some instances, the composition has less than about 10%, 9%, 8%, 7%, 6%, 5%, 4%, 3%, 2%, or 1% degradation at a storage condition for about 1, 3, 6, 9, 12, 18, 24, 26, or more months. In some instances, the storage condition is from about 20° C. to about 28° C., from about 22° C. to about 26° C., or from about 23° C. to about 27° C. In some cases, the storage condition comprises about 55%, about 60%, about 65%, about 70%, about 75%, or about 80% humidity.

In some instances, the solution less than about 10%, 9%, 8%, 7%, 6%, 5%, 4%, 3%, 2%, or 1% degradation at a storage condition. In some instances, the solution has less than about 10%, 9%, 8%, 7%, 6%, 5%, 4%, 3%, 2%, or 1% degradation at a storage condition for about 1, 3, 6, 9, 12, 18, 24, 26, or more months. In some instances, the storage condition is from about 20° C. to about 28° C., from about 22° C. to about 26° C., or from about 23° C. to about 27° C. In some cases, the storage condition comprises about 55%, about 60%, about 65%, about 70%, about 75%, or about 80% humidity.

In some embodiments, the liquid filling of the soft gelatin capsule has less than about 10%, 9%, 8%, 7%, 6%, 5%, 4%, 3%, 2%, or 1% degradation at a storage condition. In some instances, the liquid filling of the soft gelatin capsule has less than about 10%, 9%, 8%, 7%, 6%, 5%, 4%, 3%, 2%, or 1% degradation at a storage condition for about 1, 3, 6, 9, 12, 18, 24, 26, or more months. In some instances, the storage condition is from about 20° C. to about 28° C., from about 22° C. to about 26° C., or from about 23° C. to about 27° C. In some cases, the storage condition comprises about 55%, about 60%, about 65%, about 70%, about 75%, or about 80% humidity.

In some embodiments, the soft gelatin capsule has a uniformity of dosage unit. As utilized herein, the term “uniformity of dosage unit” refers to the degree of uniformity in the amount of the drug substance among dosage units. In some instances, the uniformity of dosage unit is measured by weight variation. In other instances, the uniformity of dosage unit is measured by content uniformity. In some instances, the assay for determining the uniformity of dosage unit by weight variation is in accordance to the method detailed in the U.S. Pharmaceopeia (USP) <905>. In one instance based on the methods of USP <905>, the assay comprises weighting about 10 capsules individually to obtain their gross weight. Then open each capsule to remove the liquid filling from each capsule. Next, each shell is then dried for a period of about 30 minutes prior to weighting the dried shells to calculate the net weight of the content. Calculate the drug substance content as a percentage of label claim from the net weight of the product removed from each capsule and the result of the assay for label claim described above.

In some embodiments, the composition, the solution, or the liquid filing of the soft gelatin capsule has a residual solvent of less than 5000 ppm. As utilized herein, the term “residual solvent” refers to one or more organic volatile chemicals that are used or produced in the manufacturing of drug substances, excipients, agents, or in the preparation of drug products. As used herein, the term “organic solvent” refers to carbon-based solvents or solvents comprising at least one carbon atom in its structure. The term “volatile” as used herein refers to organic solvents that evaporates at a temperature of from about 22° C. to about 27° C. and at 1 standard atmosphere (atm). In some instances, the residual solvent is calculated based on the method detailed in USP <467>. In some cases, the residual solvent is less than 4500 ppm, less than 4000 ppm, less than 3500 ppm, less than 3000 ppm, less than 2500 ppm, less than 2000 ppm, less than 1500 ppm, less than 1000 ppm, less than 500 ppm, less than 400 ppm, less than 300 ppm, less than 200 ppm, less than 100 ppm, or less than 50 ppm. In some cases, the total residual solvent is less than 5000 ppm.

In some embodiments, the soft gelatin capsule has a residual solvent of less than 5000 ppm.

In some embodiments, a quantitative evaluation of microbial content of the composition or the solution is performed. This quantitative evaluation can be referred to sometimes as either a microbial bioburden testing or a microbial limits testing. In some instances, the testing is carried out in accordance to the method detailed in USP <61> and/or USP <62>. In some instances, the USP <61> test provides enumeration of mesophilic bacteria and fungi that may grow under aerobic conditions. In some cases, the aerobic bacteria is NMT (not more than) 1000 cfu/g (colony-forming unit per gram). In some cases, the aerobic bacteria is NMT 900 cfu/g, 800 cfu/g, 700 cfu/g, 600 cfu/g, 500 cfu/g, 400 cfu/g, 300 cfu/g, 200 cfu/g, or 100 cfu/g. In some cases, the fungi (e.g., yeast and/or mold) is NMT 100 cfu/g. In some cases, the fungi is NMT 80 cfu/g, 50 cfu/g, 30 cfu/g, or 10 cfu/g.

The USP <62> test determines the presence or absence of the following microorganisms: Escherichia coli, Salmonella species, Staphylococcus aureus, Pseudomonas aeruginosa, and bile-tolerant Gram-negative bacteria such as Candida albicans, Clostridium species, and/or B. cepacia complex (Bcc). In some cases, one or more microorganisms are not detected in the composition or the solution. In some cases, one or more of Escherichia coli, Salmonella species, Staphylococcus aureus, Pseudomonas aeruginosa, or bile-tolerant Gram-negative bacteria selected from Candida albicans, Clostridium species, or B. cepacia complex (Bcc) are not detected in the composition or the solution.

In some embodiments, a quantitative evaluation of microbial content of the soft gelatin capsule is performed. In some instances, the testing is carried out in accordance to the method detailed in USP <61> and/or USP <62>. In some cases, the aerobic bacteria is NMT (not more than) 1000 cfu/g (colony-forming unit per gram). In some cases, the aerobic bacteria is NMT 900 cfu/g, 800 cfu/g, 700 cfu/g, 600 cfu/g, 500 cfu/g, 400 cfu/g, 300 cfu/g, 200 cfu/g, or 100 cfu/g. In some cases, the fungi (e.g., yeast and/or mold) is NMT 100 cfu/g. In some cases, the fungi is NMT 80 cfu/g, 50 cfu/g, 30 cfu/g, or 10 cfu/g. In some cases, the USP <62> test determines the presence or absence of the following microorganisms: Escherichia coli, Salmonella species, Staphylococcus aureus, Pseudomonas aeruginosa, and bile-tolerant Gram-negative bacteria such as Candida albicans, Clostridium species, and/or B. cepacia complex (Bcc). In some cases, one or more microorganisms are not detected in the soft gelatin capsule. In some cases, one or more of Escherichia coli, Salmonella species, Staphylococcus aureus, Pseudomonas aeruginosa, or bile-tolerant Gram-negative bacteria selected from Candida albicans, Clostridium species, or B. cepacia complex (Bcc) are not detected in the soft gelatin capsule.

### Methods of Uses

In certain embodiments, disclosed herein is a method of administering 17-HPC to a subject or to treat a disease or condition in a subject in need thereof. In some instances, the method comprises administering to the subject a composition, a solution, or a soft gelatin capsule described above.

In certain embodiment, also described herein is a method of reducing an elevated IL-17 expression, IL-2 expression, IL-4 expression, or a combination thereof and/or p38 mitogen activated protein kinase activity in a subject in need thereof. In some instances, the method comprises administering to the subject a composition, a solution, or a soft gelatin capsule described above. In some cases, administration of the composition, the solution, or the soft gelatin capsule reduces the IL-17 expression, IL-2 expression, IL-4 expression, or a combination thereof, and/or p38 mitogen activated protein kinase activity in the subject. In some cases, the composition, the solution, or the soft gelatin capsule induces a decrease in IL-2 expression, IL-4 expression, or a combination thereof in the subject. In some cases, the composition, the solution, or the soft gelatin capsule induces a decrease in IL-17 expression in the subject. In some cases, the composition, the solution, or the soft gelatin capsule induces a decrease in p38 MAPK phosphorylation. In some cases, the composition, the solution, or the soft gelatin capsule reduces steroid resistance in the subject. In some cases, the composition, the solution, or the soft gelatin capsule reverses glucocorticoid resistance in the subject.

In certain embodiments, also disclosed herein is a method of treating a subject selected for therapy, comprising (a) detecting an elevated level of IL-17 in a sample obtained from the subject and (b) administering to the subject having an elevated level of IL-17 as compared to a level of IL-17 in a subject having a predetermined range of IL-17 a composition, a solution, or a soft gelatin capsule described above.

In some instances, the disease or condition comprises a glucocorticoid insensitive disease or condition. In some instances, the disease or condition is associated with an elevated IL-17 expression or the subject exhibits an elevated IL-17 level.

In some instances, the disease or condition is associated with an elevated p38 mitogen activated protein kinase activity or the subject exhibits an elevated p38 mitogen activity.

In some instances, the disease or condition is chronic obstructive pulmonary disease, asthma, obliterative bronchitis, bronchiectasis, cystic fibrosis, sarcoidosis, eosinophilic granuloma, respiratory bronchiolitis interstitial lung disease, or emphesyma.

In some embodiments, the disease or condition is idiopathic interstitial pneumonias (IIPs). Idiopathic interstitial pneumonias are a group of interstitial lung diseases of unknown etiology that share similar clinical and radiologic features and are distinguished primarily by the histopathologic patterns of lung biopsy. In some instances, the IIPs are further classified into four groups: chronic fibrosing IIPs: idiopathic pulmonary fibrosis (IPF), and idiopathic non-specific interstitial pneumonia (NSIP); smoking related IIPs: respiratory bronchiolitis-associated interstitial lung disease (RB-ILD) and desquamative interstitial pneumonia (DIP); acute and subacute IIPs: acute interstitial pneumonia (AIP) and cryptogenic organizing pneumonia (COP); and rare IIPs: idiopathic pleuroparenchymal fibroelastosis (IPPFE) and lymphoid interstitial pneumonia (LIP). In some instances, the IIPs comprise idiopathic nonspecific interstitial pneumonitis (NSIP), desquamative interstitial pneumonia (DIP), cryptogenic organizing pneumonia (COP), lymphoid interstitial pneumonia (LIP), or idiopathic pleuroparenchymal fibroelastosis (IPPFE).

In some embodiments, the disease or condition is an inflammatory bowel disease (IBD). In some instances, the IBD comprises Crohn's disease (e.g., ileocolitis/ileoceceal Crohn's disease, ileitis, gastroduodenal Crohn's disease, jejunoileitis, or Crohn's granulomatous colitis), ulcerative colitis, indeterminate colitis, microscopic colitis, or diversion colitis. In some cases, the disease or condition is Crohn's disease. In some cases, the disease or condition is ulcerative colitis.

In some embodiments, about 120 mg, 250 mg, 360 mg, 720 mg, 750 mg, 1000 mg, or 1500 mg of 17-HPC is administered to the subject per day. In some instances, about 120 mg, 360 mg, or 720 mg of 17-HPC is administered to the subject per day. In some instances, about 360 mg of 17-HPC is administered to the subject per day. In some instances, about 720 mg of 17-HPC is administered to the subject per day.

In some instances, from about 6 mg/kg to about 12 mg/kg of 17-HPC is administered to the subject per day. In some instances, about 6 mg/kg of 17-HPC is administered to the subject per day. In some instances, about 12 mg/kg of 17-HPC is administered to the subject per day

In some instances, the composition, the solution, or the soft gelatin capsule is administered to the subject for at least 1 month, 2 months, 3 months, 4 months, 5 months, 6 months, 1 year, 1.5 year, 2 years, or more.

In some instances, a subject after administered the composition, the solution, or the soft gelatin capsule has a blood level (Cmax) of from about 0 ng/mL to about 900 ng/mL, from about 10 ng/mL to about 900 ng/mL, from about 10 ng/mL to about 800 ng/mL, from about 10 ng/mL to about 700 ng/mL, from about 10 ng/mL to about 500 ng/mL, from about 50 ng/mL to about 900 ng/mL, from about 100 ng/mL to about 900 ng/mL, or from about 200 ng/mL to about 900 ng/mL. In some instances, the blood level (Cmax) is reached after a single administration of up to or about 750 mg of 17-HPC.

Methods of Treating a Respiratory Disease or Condition Associated with or Induced by a Pathogen

In certain embodiments, disclosed herein is a method of treating a respiratory disease or condition associated with or induced by a pathogen in a subject in need thereof. In some embodiments, the method comprises administering to the subject a composition comprising 17-alpha hydroxyprogesterone caproate (17-HPC), one or more solubilizing agents, and one or more lipophilic agents to treat the respiratory disease in the subject. In some instances, the composition comprises one, two, three, four, or more solubilizing agents and one, two, three, four, or more lipophilic agents. In some instances, the composition comprises 17-HPC and a 2-component solvent system. In some instances, the solubilizing agent comprises benzyl benzoate, diethylene glycol monoethyl ether, propylene glycol monolaurate, glyceryl monocaprylate, propylene glycol monocaprylate, or oleic acid. In some instances, the lipophilic excipient comprises macrogolglycerol ricinolate, polysorbate 80, caprylocaproyl polyoxyl-8 glycerides, glyceryl monooleate, glyceryl monolinoleate, PEG 15 hydroxystearate, PEG 40 hydrogenated castor oil, PEG 35 castor oil, or olive oil.

In some embodiments, the pathogen is a virus. In some instances, the virus comprises a coronavirus. In some instances, the coronavirus is an alpha-type coronavirus or a beta-type coronavirus. In some instances, the virus comprises a pathogenic strain. In some instances, the coronavirus is 229E, NL63, OC43, HKU1, MERS-CoV, SARS-CoV, or SARS-CoV-2.

In some embodiments, the virus comprises an influenza virus, cytomegalovirus, Epstein-Barr virus, variola virus, Ebola, dengue, Measles virus, mumps virus, or rubella virus. In some instances, the influenza virus is influenza A virus.

In some embodiments, the pathogen is a bacterium, a fungus, a protozoan, or a parasite. In some instances, the bacterium is Francisella tularensis, Corynebacterium diphtheria, Legionella pneumophila, Streptococcus pneumoniae, Mycobacterium tuberculosis, Bordetella pertussis, Bacillu anthracis, Chlamydia psittaci, Coxiella burnetti, Francisella tularensis, or from the genus Brucella.

In some instances, the protozoan is Plasmodium falciparum.

In some instances, the respiratory disease is caused by group A streptococcus (GAS). In some instances, the GAS comprises Streptococcus pyogenes or Streptococcus dysgalactiae.

In some instances, the respiratory disease or condition is a lower respiratory disease or condition. In some instances, the respiratory disease or condition is pneumonia. In some instances, the respiratory disease or condition is a SARS-CoV-2 induced pneumonia.

In some instances, the subject further has organ failure, optionally multiple organ failure.

In some instances, the subject has an elevated level of one or more mediators associated with cytokine storm. In some embodiments, the one or more mediators comprise a proinflammatory cytokine. In some instances, the proinflammatory cytokine comprises TNF-α, IL-1α, IL-1β, IL-2, IL-5, IL-6, IL-9, IL-12 (p70), IL-17 (optionally IL-17A), IFN-γ, TGF-β, granulocyte/macrophage-colony-stimulating factor (GM-CSF), macrophage colony stimulating factor (M-CSF), granulocyte-colony stimulating factor (G-CSF), or reactive oxygen species (ROS).

In some embodiments, the one or more mediators comprise a proinflammatory chemokine. In some instances, the proinflammatory chemokine comprises CCL2, CCL3, CCL-5, IL-8, IFN-γ-induced protein 10 (IP-10), cutaneous T-cell-attracting chemokine (CTACK), monokine induced gamma interferon (MIG), hepatocyte growth factor (HGF), macrophage inflammatory protein 1α (MIP-1α), macrophage inflammatory protein 1β (MIP-1β), monocyte chemoattractant protein-1 (MCP-1), monocyte chematoctic protein-3 (MCP-3), platelet-derived growth factor (PDGF), regulated upon activation normal T cell expressed and secreted (RANTES), or vascular endothelial growth factor (VEGF).

In some embodiments, the one or more mediators comprise an anti-inflammatory cytokine. In some instances, the anti-inflammatory cytokine comprises IL-4, IL-10, IL-13, or fibroblast growth factor (FGF).

In some embodiments, the one or more mediators comprise a Type I IFN, a Type II IFN, or a Type III IFN.

In some embodiments, the subject has an elevated level of the one or more mediators. In some embodiments, the one or more mediators comprise IL-1, IL-2, IL-4, IL-6, IL-17, TNF-α, or a combination thereof. In some embodiments, the one or more mediators comprise TL-1RA, IL-2R, or a combination hereof. In some embodiments, the one or more mediators comprise IL-2, IL-7, G-CSF, CXCL10, MCP-1, MIP-1α, TNF-α, IL-6, or a combination thereof. In some embodiments, the one or more mediators comprise IL-2R, IL-6, or a combination thereof. In some embodiments, the one or more mediators comprise IL-1B, IFN-γ, IP-10, MCP-1, or a combination thereof. In some embodiments, the one or more mediators comprise IL-4, IL-10, or a combination thereof. In some embodiments, the one or more mediators comprise G-CSF, IP-10, MCP-1, MIP1A, TNF-α, or a combination thereof. In some embodiments, the one or more mediators comprise IL-2, IL-7, IL-10, G-CSF, IP10, MCP1, MIP1A, TNF-α, or a combination thereof. In some embodiments, the one or more mediators comprise IL-6. In some embodiments, the one or more mediators comprise D-dimers. In some embodiments, the one or more mediators comprise IFN-γ, IL-1RA, IL-2RA, IL-6, IL-10, IL-18, HGF, MCP-3, MIG, M-CSF, G-CSF, MIG-1a, CTACK, IP-10, or a combination thereof. In some embodiments, the level is a serum level. In some embodiments, the level is an expression level.

In some embodiments, 17-HPC decreases the level of one or more mediators in the subject. In some embodiments, 17-HPC decreases the level of IL-1, IL-2, IL-4, IL-6, IL-17, TNF-α, or a combination thereof. In some instances, 17-HPC decreases the level of IL-1. In some instances, 17-HPC decreases the level of IL-2. In some instances, 17-HPC decreases the level of IL-4. In some instances, 17-HPC decreases the level of IL-6. In some instances, 17-HPC decreases the level of IL-17. In some instances, 17-HPC decreases the level of TNF-α. In some cases, 17-HPC decreases the level of IL-1, IL-2, IL-4, IL-6, IL-17, TNF-α, or a combination thereof, by about 1-fold, 2-fold, 3-fold, 4-fold, 5-fold, 6-fold, 7-fold, 8-fold, 9-fold, 10-fold, 20-fold, 30-fold, 50-fold, 100-fold, 200-fold, 500-fold, or more. In some cases, the decrease in level of the one or more mediators is compared to that of their respective elevated level prior to administration of the 17-HPC.

In some embodiments, the subject has an elevated level of C-reactive protein (CRP), ferritin, procalcitonin, neopterin, S100 proteins, ADA2, or CD163, or a combination thereof. In some instances, the elevated level is a serum level.

In some embodiments, the subject has a decreased level of fibrinogen.

Methods of Modulating a Mediator Associated with Cytokine Storm

In certain embodiments, disclosed herein is a method of treating a cytokine release syndrome (CRS) in a subject in need thereof. In some instances, the method comprises administering to the subject a composition comprising 17-alpha hydroxyprogesterone caproate (17-HPC), one or more solubilizing agents, and one or more lipophilic agents to treat the cytokine release syndrome in the subject. In some cases, the subject has an elevated level of one or more mediators associated with cytokine storm as compared to a predetermined level. In some instances, the composition comprises one, two, three, four, or more solubilizing agents and one, two, three, four, or more lipophilic agents. In some instances, the composition comprises 17-HPC and a 2-component solvent system. In some instances, the solubilizing agent comprises benzyl benzoate, diethylene glycol monoethyl ether, propylene glycol monolaurate, glyceryl monocaprylate, propylene glycol monocaprylate, or oleic acid. In some instances, the lipophilic excipient comprises macrogolglycerol ricinolate, polysorbate 80, caprylocaproyl polyoxyl-8 glycerides, glyceryl monooleate, glyceryl monolinoleate, PEG 15 hydroxystearate, PEG 40 hydrogenated castor oil, PEG 35 castor oil, or olive oil.

In certain embodiments, also disclosed herein is a method of modulating the level of one or more mediators of cytokine storm in a subject in need thereof, comprising administering to the subject a composition comprising 17-alpha hydroxyprogesterone caproate (17-HPC), one or more solubilizing agents, and one or more lipophilic agents to modulate the level of the one or more mediators. In some instances, the composition comprises one, two, three, four, or more solubilizing agents and one, two, three, four, or more lipophilic agents. In some instances, the composition comprises 17-HPC and a 2-component solvent system. In some instances, the solubilizing agent comprises benzyl benzoate, diethylene glycol monoethyl ether, propylene glycol monolaurate, glyceryl monocaprylate, propylene glycol monocaprylate, or oleic acid. In some instances, the lipophilic excipient comprises macrogolglycerol ricinolate, polysorbate 80, caprylocaproyl polyoxyl-8 glycerides, glyceryl monooleate, glyceryl monolinoleate, PEG 15 hydroxystearate, PEG 40 hydrogenated castor oil, PEG 35 castor oil, or olive oil.

In certain embodiments, additionally disclosed herein is a method of treating a subject selected for therapy, comprising administering to the subject having an elevated level of a mediator associated with cytokine storm as compared to a predetermined level of the mediator a composition comprising 17-alpha hydroxyprogesterone caproate (17-HPC), one or more solubilizing agents, and one or more lipophilic agents. In some instances, the subject is selected for the therapy by a method comprising detecting an elevated level of a mediator associated with cytokine storm in a sample isolated from the subject. In some instances, the composition comprises one, two, three, four, or more solubilizing agents and one, two, three, four, or more lipophilic agents. In some instances, the composition comprises 17-HPC and a 2-component solvent system. In some instances, the solubilizing agent comprises benzyl benzoate, diethylene glycol monoethyl ether, propylene glycol monolaurate, glyceryl monocaprylate, propylene glycol monocaprylate, or oleic acid. In some instances, the lipophilic excipient comprises macrogolglycerol ricinolate, polysorbate 80, caprylocaproyl polyoxyl-8 glycerides, glyceryl monooleate, glyceryl monolinoleate, PEG 15 hydroxystearate, PEG 40 hydrogenated castor oil, PEG 35 castor oil, or olive oil.

In some embodiments, the one or more mediators comprise a proinflammatory cytokine. In some instances, the proinflammatory cytokine comprises TNF-α, IL-1α, IL-1β, IL-2, IL-5, IL-6, IL-9, IL-12 (p70), IL-17 (optionally IL-17A), IFN-γ, TGF-β, granulocyte/macrophage-colony-stimulating factor (GM-CSF), macrophage colony stimulating factor (M-CSF), granulocyte-colony stimulating factor (G-CSF), or reactive oxygen species (ROS).

In some embodiments, the one or more mediators comprise a proinflammatory chemokine. In some instances, the proinflammatory chemokine comprises CCL2, CCL3, CCL-5, IL-8, IFN-γ-induced protein 10 (IP-10), cutaneous T-cell-attracting chemokine (CTACK), monokine induced gamma interferon (MIG), hepatocyte growth factor (HGF), macrophage inflammatory protein 1a (MIP-1α), macrophage inflammatory protein 1β (MIP-1β), monocyte chemoattractant protein-1 (MCP-1), monocyte chematoctic protein-3 (MCP-3), platelet-derived growth factor (PDGF), regulated upon activation normal T cell expressed and secreted (RANTES), or vascular endothelial growth factor (VEGF).

In some embodiments, the one or more mediators comprise an anti-inflammatory cytokine. In some instances, the anti-inflammatory cytokine comprises IL-4, IL-10, IL-13, or fibroblast growth factor (FGF).

In some embodiments, the one or more mediators comprise a Type I IFN, a Type II IFN, or a Type III IFN.

In some embodiments, the subject has an elevated level of the one or more mediators. In some embodiments, the one or more mediators comprise IL-1, IL-2, IL-4, IL-6, IL-17, TNF-α, or a combination thereof. In some embodiments, the one or more mediators comprise TL-1RA, IL-2R, or a combination hereof. In some embodiments, the one or more mediators comprise IL-2, IL-7, G-CSF, CXCL10, MCP-1, MIP-1α, TNF-α, IL-6, or a combination thereof. In some embodiments, the one or more mediators comprise IL-2R, IL-6, or a combination thereof. In some embodiments, the one or more mediators comprise IL-1B, IFN-γ, IP-10, MCP-1, or a combination thereof. In some embodiments, the one or more mediators comprise IL-4, IL-10, or a combination thereof. In some embodiments, the one or more mediators comprise G-CSF, IP-10, MCP-1, MIP1A, TNF-α, or a combination thereof. In some embodiments, the one or more mediators comprise IL-2, IL-7, IL-10, G-CSF, IP10, MCP1, MIP1A, TNF-α, or a combination thereof. In some embodiments, the one or more mediators comprise IL-6. In some embodiments, the one or more mediators comprise D-dimers. In some embodiments, the one or more mediators comprise IFN-γ, IL-1RA, IL-2RA, IL-6, IL-10, IL-18, HGF, MCP-3, MIG, M-CSF, G-CSF, MIG-1a, CTACK, IP-10, or a combination thereof. In some embodiments, the level is a serum level. In some embodiments, the level is an expression level.

In some embodiments, 17-HPC decreases the level of one or more mediators in the subject. In some embodiments, 17-HPC decreases the level of IL-1, IL-2, IL-4, IL-6, IL-17, TNF-α, or a combination thereof. In some instances, 17-HPC decreases the level of IL-1. In some instances, 17-HPC decreases the level of IL-2. In some instances, 17-HPC decreases the level of IL-4. In some instances, 17-HPC decreases the level of IL-6. In some instances, 17-HPC decreases the level of IL-17. In some instances, 17-HPC decreases the level of TNF-α. In some cases, 17-HPC decreases the level of IL-1, IL-2, IL-4, IL-6, IL-17, TNF-α, or a combination thereof, by about 1-fold, 2-fold, 3-fold, 4-fold, 5-fold, 6-fold, 7-fold, 8-fold, 9-fold, 10-fold, 20-fold, 30-fold, 50-fold, 100-fold, 200-fold, 500-fold, or more. In some cases, the decrease in level of the one or more mediators is compared to that of their respective elevated level prior to administration of the 17-HPC.

In some embodiments, the subject has an elevated level of C-reactive protein (CRP), ferritin, procalcitonin, neopterin, S100 proteins, ADA2, or CD163, or a combination thereof. In some instances, the elevated level is a serum level.

In some embodiments, the subject has a decreased level of fibrinogen.

In some embodiments, the CRS or the cytokine storm is associated with or induced by a pathogen. In some instances, the pathogen is a virus, a bacterium, a fungus, a protozoan, or a parasite. In some instances, the virus is a coronavirus. In some instances, the coronavirus is an alpha-type coronavirus or a beta-type coronavirus. In some instances, the coronavirus is a pathogenic strain. In some instances, the coronavirus is 229E, NL63, OC43, HKU1, MERS-CoV, SARS-CoV, or SARS-CoV-2.

In some instances, the virus is an influenza virus, cytomegalovirus, Epstein-Barr virus, variola virus, Ebola, dengue, Measles virus, mumps virus, or rubella virus. In some instances, the influenza virus is influenza A virus.

In some instances, the bacterium is Francisella tularensis, Corynebacterium diphtheria, Legionella pneumophila, Streptococcus pneumoniae, Mycobacterium tuberculosis, Bordetella pertussis, Bacillu anthracis, Chlamydia psittaci, Coxiella burnetti, Francisella tularensis, or from the genus Brucella.

In some instances, the protozoan is Plasmodium falciparum.

In some instances, the CRS or the cytokine storm is caused by group A streptococcus (GAS). In some instances, the GAS comprises Streptococcus pyogenes or Streptococcus dysgalactiae.

In some embodiments, the CRS or the cytokine storm is associated with or induced by a non-infectious disease or condition. In some instances, the non-infectious disease or condition is a graft-versus-host disease, pancreatitis, or multiple organ dysfunction syndrome.

In some instances, the CRS or the cytokine storm is induced by a chimeric antigen receptor (CAR) treatment, optionally a CAR T-cell treatment or CAR NK treatment.

In some embodiments, the subject has or develops acute respiratory distress syndrome (ARDS), acute lung injury (ALI), increased capillary permeability syndrome, hemophagocytic lymphohistiocytosis (HLH), or a combination thereof.

In some embodiments, the subject has or develops lymphocytopenia.

In some embodiments, the subject has or develops a respiratory disease, optionally a lower respiratory disease. In some instances, the subject has or develops pneumonia, optionally a pathogen induced pneumonia, further optionally SARS-CoV-2 induced pneumonia. In some instances, the subject has or develops organ failure, optionally multiple organ failure.

In some embodiments, the subject has an underlying disease or condition. In some instances, the underlying disease or condition is hypertension, cardiovascular disease, diabetes, or chronic lung disease. In some instances, the subject is suffering from or predisposed to suffer from a glucocorticoid insensitive disease or condition. In some instances, the subject is suffering from or predisposed to suffer from a disease or condition associated with an elevated p38 mitogen activated protein kinase activity. In some instances, the subject is suffering from or predisposed to suffer from chronic obstructive pulmonary disease, asthma, obliterative bronchitis, bronchiectasis, cystic fibrosis, sarcoidosis, eosinophilic granuloma, respiratory bronchiolitis interstitial lung disease, or emphesyma.

In some embodiments, the subject is suffering from or predisposed to suffer from idiopathic interstitial pneumonia (IIP). In some instances, the subject is suffering from or predisposed to suffer from idiopathic nonspecific interstitial pneumonitis, desquamative interstitial pneumonia, cryptogenic organizing pneumonia, lymphoid interstitial pneumonia, or idiopathic pleuroparenchymal fibroelastosis.

In some embodiments, the subject is suffering from or predisposed to suffer from an inflammatory bowel disease (IBD). In some instances, the IBD comprises Crohn's disease (e.g., ileocolitis/ileoceceal Crohn's disease, ileitis, gastroduodenal Crohn's disease, jejunoileitis, or Crohn's granulomatous colitis), ulcerative colitis, indeterminate colitis, microscopic colitis, or diversion colitis. In some cases, the subject is suffering from or predisposed to suffer from Crohn's disease. In some cases, the disease or condition is ulcerative colitis.

In some embodiments, the subject is suffering from or predisposed to suffer from acquired immunodeficiency syndrome (AIDS).

In some embodiments, the subject is suffering from or predisposed to suffer from familial hemophagocytic lymphohistiocytosis (fHLH), Griscelli syndrome, Chediak-Higashi syndrome, Hermansky-Pudlak syndrome, X-linked lymphoproliferative syndrome (XLP), macrophage activation syndrome (MAS), or malignancy-associated hemophagocytic syndromes (MAHS).

In some embodiments, the subject is suffering from or predisposed to suffer from an autoimmune disease. In some instances, the autoimmune disease is multiple sclerosis, Lupus, Sjögren's syndrome, or adult-onset Still's disease (AOSD).

In some embodiments, the subject has a secondary infection.

In some embodiments, the composition is formulated for oral administration.

In some embodiments, the composition is formulated as a soft gelatin capsule.

In some embodiments, the composition is formulated for systemic administration.

In some embodiments, the composition is formulated for local administration.

In some embodiments, the composition is formulated for parenteral administration. In some instances, the composition is formulated for intravenous, intramuscular, intraperitoneal, or subcutaneous administration.

In some embodiments, the composition is formulated as an aerosol.

In some embodiments, the composition is formulated for topical, intranasal, sublingual, buccal, or sublingual administration.

In some embodiments, the subject is a human.

Use of 17-HPC-Based Treatment with One or More Additional Therapies

In some embodiments, the method further comprises administering to the subject an additional therapeutic agent with a composition, solution, or soft gelatin capsule comprising 17-HPC. In some instances, the additional therapeutic agent is a glucocorticoid. In some instances, the additional therapeutic agent is selected from hydrocortisone, cortisone acetate, dexamethasone, prednisone, prednisolone, methylprednisolone, betamethasone, triamcinolone, beclometasone (also known as beclomethasone dipropionate), Paramethasone, fluticasone, fludrocortisone acetate, deoxycorticosterone acetate, Fluprednisolone, fluticasone propionate, budesonide, flunisolide, triamcinolone acetonide, or a combination thereof. In some instances, the additional therapeutic agent is dexamethasone (DEX). In some instances, the additional therapeutic agent is budesonide (BUD). In some instances, the additional therapeutic agent is prednisone. In some cases, the additional therapeutic agent does not comprise a composition, solution, or soft gelatin capsule comprising 17-HPC.

In some embodiments, the method further comprises administering an additional therapeutic agents or an additional therapy treatment with a composition, solution, or soft gelatin capsule comprising 17-HPC. In some instances, the additional therapeutic agent comprises a corticosteroid, a chemokine inhibitor, an IL-1 family antagonist, an IL-6R antagonist, a TNF blocker, an IFN-αβ inhibitor, an anti-IFN-γ antibody, a JAK inhibitor, an angiotensin-converting enzyme (ACE) inhibitor, a Cox-inhibitor, a nonsteroidal anti-inflamatory drug (NSAID), a type 2 taste receptor (TAS2R) ligand, a an antiviral therapeutics, or a combination thereof. In some instances, the additional therapeutic agent comprises an antiviral, an antimalarial, an immune booster, an anti-inflammatory agent, a mucolytic, an anti-coagulant, a vasodilator, or an anti-angiogenesis. In some instances, the additional therapeutic agent does not comprise a composition comprising 17-HPC.

In some embodiments, the additional therapeutic agent comprises a chemokine inhibitor. In some instances, the additional therapeutic agent comprises a broad-spectrum chemokine inhibitor (BSCI), optionally NR58-3.14.3; or an αMCP-1 antibody.

In some embodiments, the additional therapeutic agent comprises an IL-1 family antagonist. In some instances, the additional therapeutic agent comprises an antagonist of IL-1β, IL-18, or IL-33. In some cases, the additional therapeutic agent comprises Anakinra.

In some embodiments, the additional therapeutic agent comprises an IL-6R antagonist. In some instances, the additional therapeutic agent comprises Tocilizumab or Sarilumab.

In some embodiments, the additional therapeutic agent comprises a TNF blocker. In some instances, the additional therapeutic agent comprises adalimumab, certolizumab, etanercept, golimumab, or infliximab.

In some embodiments, the additional therapeutic agent comprises an IFN-αβ inhibitor. In some instances, the additional therapeutic agent comprises interferon beta-1a, interferon beta-1b, peginterferon beta-1a, or interferon alfa-n3.

In some embodiments, the additional therapeutic agent comprises an anti-IFN-γ antibody. In some instances, the additional therapeutic agent comprises Emapalumab.

In some embodiments, the additional therapeutic agent comprises a Janus kinase (JAK) inhibitor. In some instances, the additional therapeutic agent comprises fedratinib, ruxolitinib, tofacitinib, oclacitinib, baricitinib, peficitinib, upadacitinib, filgotinib, cerdulatinib, gandotinib, lestaurtinib, momelotinib, pacritinib, or abrocitinib.

In some embodiments, the additional therapeutic agent comprises an angiotensin-converting enzyme (ACE) inhibitor. In some instances, the additional therapeutic agent comprises benazepril, captopril, enalapril, fosinopril, lisinopril, moexipril, perindopril quinapril, ramipril, or trandolapril.

In some embodiments, the additional therapeutic agent comprises a Cox inhibitor. In some instances, the additional therapeutic agent comprises a nonsteroidal anti-inflammatory drug (NSAID). In some cases, the additional therapeutic agent comprises aspirin, celecoxib, diclofenac, diflunisal, etodolac, ibuprofen, indomethacin, ketoprofen, ketorolac, nabumetone, naproxen, oxaprozin, piroxicam, salsalate, sulindac, or tolmetin.

In some embodiments, the additional therapeutic agent comprises a type 2 taste receptor (TAS2R) ligand. In some instances, the additional therapeutic agent comprises a ligand to TAS2R10, TAS2R14, and/or TAS2R46. In some cases, the additional therapeutic agent comprises Diphenidol, Quinine, Chlorpheniramine, Denatonium benzoate, Parthenolide, Arborescin, Chloramphenicol, Cascarillin, Picrotoxinin, Quassin, Azathioprine, Artemorin, Papaverine, Yohimbine, Camphor, Dapsone, Strychnine, Dextromethorphan, Haloperidol, Brucine, Coumarin, Cucurbitacin B, (−)-a-Thujon, Benzoin, Famotidine, Cucurbitacin E, Cycloheximide, Erythromycin, Diphenylthiourea, Colchicine, Sodium benzoate, Diphenhydramine, Carisoprodol, Noscapine, Benzamide, Chlorhexidine, Divinylsulfoxid, Flufenamic acid, 4-Hydroxyanisol, Hydrocortisone, Orphenadrine, Tatridin B, or Artemisinin.

In some embodiments, the additional therapeutic agent comprises an antiviral therapeutics. In some instances, the additional therapeutic agent comprises lopinavir, ritonavir, Remdesivir (GS-5734), nitazoxanide, favipiravir, nafamostat, osetamivir, penciclovir/acyclovir, or ganciclovir, or a combination thereof.

In some embodiments, the additional therapeutic agent comprises chloroquine (CQ), hydroxychloroquine (HCQ), cyclosporine, tacrolimus, Ulinastatin, oxidized phospholipids (OxPL), or Sphingosine-1-phosphate (SIP) agonist.

In some embodiments, the additional therapeutic agent comprises nicotine or GTS-21.

In some embodiments, the additional therapeutic agent comprises an α-HMGB1 antibody.

In some embodiments, the additional therapeutic agent comprises statin.

In some embodiments, the additional therapeutic agent comprises baricitinib.

In some embodiments, the additional therapeutic agent comprises melatonin.

In some embodiments, the additional therapeutic agent comprises Shufeng Jiedu Capsule (SFJDC).

In some embodiments, the additional therapeutic agent comprises an approved COVID-19 vaccines by the U.S. Food and Drug Administration (FDA), by the European Commission, by the Medicines and Healthcare products Regulatory Agency (MHRA) of the United Kingdom (UK), by the National Medical Products Administration (NMPA) of the People's Republic of China, by the India Central Drugs Standard Control Organization (CDSCO), by the Ministry of Food and Drug Safety (MFDS) of South Korea, or by the Ministry of Health of the Russian Federation. In some instances, the additional therapeutic agent comprises a COVID-19 vaccine such as, but not limited to, the COVID-19 vaccine from Pfizer-BioNTech; the COVID-19 vaccine from Moderna, Inc.; the COVID-19 vaccine from Johnson & Johnson's Janssen; the COVID-19 vaccine from AstraZeneca; the Sputnik V COVID-19 vaccine; or the COVID-19 vaccines from Sinovac, Sinopharm, CanSino, or Anhui Zhifei Longcom.

In some embodiments, the additional therapeutic agent comprises one or more agents from Table 17.

TABLE 17

| | | | | Clinical Trial |
|---|---|---|---|---|
| Intervention | Type | Comparator | Phase | Registration # |
| Hydroxychloroquine | Antimalarial | Placebo | Ph IV | ChiCTR2000029559 |
| Hydroxychloroquine | Antimalarial | Supportive care | Ph IV | ChiCTR2000029740 |
| Sildenafil citrate | Vasodilator | Single arm | Ph III | NCT04304313 |
| α lipoic acid | Antioxidant | Placebo | Ph IV | ChiCTR2000029851 |
| Ebastine + Lopinavir + | Mucolytic; | Lopinavir + | Ph IV | ChiCTR2000030535 |
| Interferon α | antiviral | Interferon α | | |
| Mesenchymal stem | Cell therapy | Placebo | Ph | ChiCTR2000029990 |
| cells | | | I/II | |
| Danoprevir + | Antiviral; | Lopinavir/ritonavir; | Ph IV | NCT04291729 |
| Ritonavir + interferon | Immunomodulator | Pegasys; Novaferon; | | |
| | | Chinese medicines + | | |
| | | interferon (4 | | |
| | | arms) | | |
| Bevacizumab | Antibody | Single arm | Ph | NCT04275414 |
| biosimilar | | | II/III | |
| Tocilizumab | Antibody | Single arm | Ph II | NCT04315480 |
| Remdesivir | Antiviral | Placebo | Ph III | NCT04257656 |
| Remdesivir | Antiviral | Placebo | Ph III | NCT04252664 |
| Dipyridamole | Vasodilator | Supportive care | Ph IV | ChiCTR2000030055 |
| Low molecular weight | Anticoagulant | Supportive care | Ph IV | ChiCTR2000030946 |
| heparin | | | | |
| Bromhexine + Arbidol | Mucolytic; | Arbidol umifenovir + | N/A | NCT04273763 |
| umifenovir + | Antiviral; | Interferon α2b | | |
| Favipiravir + | Immunomodulator | | | |
| Interferon α2b | | | | |
| Favipiravir high dose; | Antiviral | N/A | Ph II | ChiCTR2000029996 |
| middle dose; low dose | | | | |
| (3 arms) | | | | |
| Levamisole + | Antiparasitic; | Lopinavir + | Ph | NCT04331470 |
| Budesonide + | Corticosteroid; | Ritonavir + | II/III | |
| Formoterol + | Bronchodilator; | Hydoxychloroquine | | |
| Lopinavir + Ritonavir + | Antiviral; | | | |
| Hydoxychloroquine | Antimalarial | | | |
| Interferon β1a + | Immunomodulator; | Hydroxychloroquine + | Ph IV | NCT04343768 |
| Hydroxychloroquine + | Antimalarial; | Lopinavir/Ritonavir | | |
| Lopinavir/Ritonavir; | Antiviral | | | |
| Interferon β1b + | | | | |
| Hydroxychloroquine + | | | | |
| Lopinavir/Ritonavir (2 | | | | |
| arms) | | | | |
| Interferon β1α + | Immunomodulator; | Lopinavir/Ritonavir + | Ph IV | NCT04350671 |
| Lopinavir/Ritonavir + | Antimalarial; | Hydroxychloroquine | | |
| Hydroxychloroquine | Antiviral | | | |
| Umifenovir + | Antiviral; | Lopinavir/Ritonavir + | Ph IV | NCT04350684 |
| Interferon β1α + | Immunomodulator; | Hydroxychloroquine | | |
| Lopinavir/Ritonavir + | Antimalarial | | | |
| Hydroxychloroquine | | | | |
| Methylprednisolone | Corticosteroid | Supportive care | Ph | NCT04244591 |
| | | | II/III | |
| DAS181 | Antiviral | Single arm | N/A | NCT04324489 |
| Hydroxychloroquine; | Antimalarial | Placebo | Ph IV | NCT04346667 |
| Chloroquine (3 arms) | | | | |
| Chloroquine | Antimalarial | Placebo | Ph II | ChiCTR2000031204 |
| phosphate | | | | |
| Thalidomide | Immunomodulator | Placebo | Ph II | NCT04273581 |
| Hydroxychloroquine; | Antimalarial | Placebo | Ph IV | NCT04351191 |
| Chloroquine (3 arms) | | | | |
| Baricitinib; Ritonavir | Anti- | Antiviral and/or | Ph III | NCT04320277 |
| | inflammatory; | hydroxychloroquine | | |
| | Antiviral | | | |
| Danoprevir + | Antiviral | Single arm | Ph IV | NCT04345276 |
| Ritonavir | | | | |
| α lipoic acid | Antioxidant | Placebo | Ph IV | ChiCTR2000030471 |
| Intravenous | Antibody | Supportive care | Ph | NCT04261426 |
| immunoglobulin | | | II/III | |
| (IVIG) | | | | |
| Hydroxychloroquine | Antimalarial | Chloroquine | Ph IV | ChiCTR2000029898 |
| | | phosphate | | |
| Hydroxychloroquine | Antimalarial | Chloroquine | Ph IV | ChiCTR2000029899 |
| | | phosphate | | |
| Bevacizumab | Antibody | Supportive care | N/A | NCT04305106 |
| biosimilar | | | | |
| Danoprevir + | Antiviral | Supportive care | N/A | ChiCTR2000030259 |
| Ritonavir | | | | |
| Tocilizumab + | Antibody | Supportive care | Ph IV | ChiCTR2000030580 |
| Adalimumab | | | | |
| PD-1 blocking | Antibody; | Supportive care | Ph II | NCT04268537 |
| antibody; Thymosin (2 | hormone | | | |
| arms) | | | | |
| Mesenchymal stem | Cell therapy | Single arm | Ph II | NCT04269525 |
| cells | | | | |
| Corticosteroid | Corticosteroid | Supportive care | Ph IV | ChiCTR2000030481 |
| Anakinra | Immunomodulator | Supportive care | Ph II | NCT04341584 |
| Eculizumab | Antibody | Supportive care | Ph II | NCT04346797 |
| Sarilumab + | Antibody; | Sarilumab | Ph | NCT04341870 |
| Azithromycin + | Antibiotic; | | II/III | |
| Hydroxychloroquine | Antimalarial | | | |
| Methylprednisolone | Corticosteroid | Supportive care | N/A | NCT04273321 |
| Remdesivir 5 days; 10 | Antiviral | N/A | Ph III | NCT04292899 |
| days (2 arms) | | | | |
| Remdesivir 5 days; 10 | Antiviral | Supportive care | Ph III | NCT04292730 |
| days (2 arms) | | | | |
| Povidone-Iodine; | Antiseptic | Placebo | Ph II | NCT04344236 |
| Chlorhexidine (2 | | | | |
| arms) | | | | |
| Favipiravir + | Antiviral; | Favipiravir; | N/A | NCT04310228 |
| Tocilizumab | Antibody | Tocilizumab (2 | | |
| | | arms) | | |
| ASC09 + oseltamivir; | Antiviral | Oseltamivir | Ph III | NCT04261270 |
| ritonavir + oseltamivir | | | | |
| (2 arms) | | | | |
| Nintedanib | Antifibrotic | Placebo | Ph II | NCT04338802 |
| Tocilizumab | Antibody | Supportive care | Ph IV | ChiCTR2000029765 |
| Ruxolitinib + | Anti- | Supportive care | Ph II | NCT04348695 |
| Simvastatin | inflammatory; | | | |
| | Vasodilator | | | |
| Hydroxychloroquine + | Antimalarial; | Hydroxychloroquine + | N/A | NCT04349592 |
| Azithromycin | Antibiotic | Placebo; Placebo | | |
| | | (2 arms) | | |
| Xiyanping + | Antiviral; | Lopinavir/ritonavir + | N/A | NCT04275388 |
| Lopinavir/ritonavir + | Immunomodulator | interferon α | | |
| interferon α | | | | |
| Tocilizumab + | Anti- | Supportive care | Ph II | NCT04335305 |
| Pembrolizumab | inflammatory; | | | |
| | Immune booster | | | |
| Mesenchymal stem | Cell therapy | Single arm | Ph I | NCT04315987 |
| cells | | | | |
| Siltuximab; | Anti-inflammatory | N/A | Ph II | NCT04329650 |
| Methylprednisolone (2 | | | | |
| arms) | | | | |
| Hydroxychloroquine | Antimalarial | Placebo | Ph I | NCT04333654 |
| Interferon α1b + | Immunomodulator; | N/A | Ph III | NCT04320238 |
| Thymosin α1; | hormone | | | |
| Interferon α1b (2 | | | | |
| arms) | | | | |
| Methylprednisolone | Corticosteroid | Supportive care | Ph | NCT04323592 |
| | | | II/III | |
| Chloroquine | Antimalarial | Supportive care | Ph IV | ChiCTR2000029992 |
| phosphate; | | | | |
| Hydroxychloroquine | | | | |
| (2 arms) | | | | |
| Hydroxychloroquine; | Antimalarial; | Placebo | Ph III | NCT04328272 |
| Hydroxychloroquine + | Antibiotic | | | |
| Azithromycin (2 arms) | | | | |
| Triazavirin | Antiviral | Placebo | Ph III | ChiCTR2000030001 |
| Danoprevir + | Antiviral | Single arm | N/A | ChiCTR2000031734 |
| Ritonavir | | | | |
| Tocilizumab | Antibody | Supportive care | Ph II | NCT04346355 |
| Angiotensin 1-7 | Vasodilator | Placebo | Ph | NCT04332666 |
| | | | II/III | |
| Colchicine | Anti-inflammatory | Standard of care | Ph III | NCT04328480 |
| Thalidomide | Immunomodulator | Placebo | Ph II | NCT04273529 |
| Lopinavir/ritonavir; | Antiviral | Supportive care | Ph IV | NCT04252885 |
| Arbidol umifenovir (2 | | | | |
| arms) | | | | |
| Colchicine | Anti-inflammatory | Standard of care | Ph II | NCT04322565 |
| Leflunomide | Immunomodulator | Placebo | Ph III | ChiCTR2000030058 |
| Interferon α1β | Immunomodulator | Supportive care | Ph I | NCT04293887 |
| Chloroquine | Antimalarial | Supportive care | Ph IV | ChiCTR2000030718 |
| phosphate | | | | |
| Nitazoxanide | Antiviral | Placebo | N/A | NCT04348409 |
| Hydroxychloroquine + | Antimalarial; | Single arm | Ph I | NCT04329572 |
| Azithromycin | Antibiotic | | | |
| Tocilizumab | Antibody | Single arm | Ph II | ChiCTR2000030196 |
| Interferon α1β | Immunomodulator | Placebo | Ph IV | ChiCTR2000029989 |
| Mesenchymal stem | Cell therapy | Placebo | Ph II | ChiCTR2000030138 |
| cells | | | | |
| ASC09/ritonavir; | Antiviral | N/A | N/A | NCT04261907 |
| Lopinavir/ritonavir (2 | | | | |
| arms) | | | | |
| Favipiravir + | Antiviral; | Favipiravir; | Ph IV | ChiCTR2000030894 |
| Tocilizumab | Antibody | Tocilizumab (2 | | |
| | | arms) | | |
| Mesenchymal stem | Anti-inflammatory | Single arm | Ph I | NCT04276987 |
| cell-derived exosomes | | | | |
| Ulinastatin | Anti-inflammatory | Supportive care | Ph IV | ChiCTR2000030779 |
| Chloroquine | Antimalarial | Single arm | Ph IV | ChiCTR2000029975 |
| phosphate | | | | |
| Chloroquine | Antimalarial | Supportive care | Ph IV | ChiCTR2000029988 |
| phosphate | | | | |
| Dexamethasone + | Corticosteroid; | Hydroxychloroquine | Ph III | NCT04347980 |
| Hydroxychloroquine | Antimalarial | | | |
| Intravenous | Antibody | Placebo | Ph III | NCT04350580 |
| immunoglobulin | | | | |
| (IVIG) | | | | |
| Ruxolitinib | Anti-inflammatory | Single arm | Ph | NCT04334044 |
| | | | I/II | |
| Tacrolimus + | Immunomodulator; | Supportive care | Ph III | NCT04341038 |
| Methylprednisolone | Anti-inflammatory | | | |
| Favipiravir | Antiviral | Supportive care | N/A | NCT04333589 |
| TD-0903 (2 arms) | Anti-inflammatory | Placebo | Ph I | NCT04350736 |
| Arbidol umifenovir; | Antiviral | N/A | Ph IV | NCT04255017 |
| Oseltamivir; | | | | |
| Lopinavir/ritonavir (3 | | | | |
| arms) | | | | |
| Arbidol umifenovir; | Antiviral; | Arbidol umifenovir | Ph IV | NCT04254874 |
| peginterferon alfa-2b | Immunomodulator | | | |
| Methylprednisolone | Corticosteroid | N/A | Ph IV | NCT04263402 |
| low dose; high dose (2 | | | | |
| arms) | | | | |
| Hydroxychloroquine | Antimalarial | Placebo | Ph II | NCT04343677 |
| (3 arms) | | | | |
| Hydroxychloroquine | Antimalarial | Placebo | Ph III | NCT04346329 |
| Hydroxychloroquine | Antimalarial | Single arm | Ph I | NCT04351620 |
| Luvox fluvoxamine | Immunomodulator | Placebo | Ph II | NCT04342663 |
| Piclidenoson | Anti-inflammatory | Supportive care | Ph II | NCT04333472 |
| Hydroxychloroquine + | Antimalarial; | Bromhexine | Ph I | NCT04340349 |
| Bromhexine | Mucolytic | | | |
| Allogeneic natural | Cell therapy | Single arm | Ph | NCT04344548 |
| killer cells | | | I/II | |
| Chloroquine analog; | Antimalarial; | Standard of care | Ph II | NCT04333914 |
| Nivolumab; | Immune booster; | | | |
| Tocilizumab (3 arms) | Anti-inflammatory | | | |
| Hydroxychloroquine + | Antimalarial; Anti- | Single arm | Ph | NCT04344457 |
| Indomethacin + | inflammatory; | | I/II | |
| Zithromax | Antibiotic | | | |
| Tofacitinib | Anti-inflammatory | Single arm | Ph II | NCT04332042 |
| Favipiravir + | Antiviral; | Favipiravir; Placebo | Ph | ChiCTR2000030987 |
| Chloroquine | Antimalarial | (2 arms) | II/III | |
| phosphate | | | | |
| Hydroxychloroquine + | Antimalarial; | Single arm | Ph I | NCT04348474 |
| Azithromycin | Antibiotic | | | |
| Inactivated | Cell therapy | Placebo | N/A | NCT04347174 |
| Mycobacterium w | | | | |
| Interferon α1β | Immunomodulator | Supportive care | Ph IV | ChiCTR2000030013 |
| Hydroxychloroquine | Antimalarial | Placebo | Ph III | NCT04341441 |
| daily; weekly (2 arms) | | | | |
| Lopinavir/ritonavir + | Antiviral | Lopinavir/ritonavir | N/A | ChiCTR2000029468 |
| emtricitabine/tenofovir | | | | |
| alafenamide fumarate | | | | |
| Alvesco ciclesonide; | Antimalarial; | Standard of care | Ph II | NCT04330586 |
| Alvesco ciclesonide + | Corticosteroid | | | |
| Hydroxychloroquine | | | | |
| (2 arms) | | | | |
| Emtricitabine + | Antiviral; | Placebo | Ph III | NCT04334928 |
| Tenofovir disoproxil + | Antimalarial | | | |
| Hydroxychloroquine; | | | | |
| Emtricitabine + | | | | |
| Tenofovir disoproxil; | | | | |
| Hydroxychloroquine | | | | |
| (3 arms) | | | | |
| Mesenchymal stem | Cell therapy | Placebo | Ph | NCT04339660 |
| cells | | | I/II | |
| Danoprevir + | Antiviral | Supportive care | N/A | ChiCTR2000030472 |
| Ritonavir | | | | |
| Mesenchymal stem | Cell therapy | Single arm | Ph I | NCT04313322 |
| cells | | | | |
| Novaferon + Arbidol | Antiviral; | Arbidol umifenovir; | Ph IV | ChiCTR2000029573 |
| umifenovir; | Immunomodulator | Lopinavir/ritonavir | | |
| Novaferon + | | (3 arms) | | |
| Lopinavir/ritonavir (3 | | | | |
| arms) | | | | |
| Escin oral; parenteral | Anti-inflammatory | Supportive care | Ph | NCT04322344 |
| (2 arms) | | | II/III | |
| Tradjenta linagliptin | Antiviral | Insulin | Ph IV | NCT04341935 |
| Mesenchymal stem | Cell therapy | Placebo | N/A | NCT04273646 |
| cells | | | | |
| Azvudine | Antiviral | Single arm | N/A | ChiCTR2000030041 |

In some embodiments, the additional therapeutic agent comprises an antiviral selected from arbidol umifenovir, ASCO9, azvudine, danoprevir, danoprevir, DAS181, emtricitabine, favipiravir, lopinavir, lopinavir/ritonavir, oseltamivir, ritonavir, remdesivir, tenofovir, triazavirin, or xiyanping.

In some embodiments, the additional therapeutic agent comprises bevacizumab or bevacizumab biosimilar, interferon α, interferon α1b, interferon α2b, IVIG, nivolumab, novaferon, peginterferon α2b, pembrolizumab, thymosin cal.

In some embodiments, the additional therapeutic agent comprises an anti-inflammatories selected from adalimumab, alvesco ciclesonide, baricitinib, budesonide, colchicine, leflunomide, methylprednisolone, MSC-derived exosomes, piclidenoson, ruxolitinib, siltuximab, thalidomide, tocilizumab, tofacitinib, or ulinastatin.

In some embodiments, the additional therapeutic agent comprises a mucolytic selected from bromhexine or ebastine.

In some embodiments, the additional therapeutic agent comprises an anticoagulant. In some instances, the anticoagulant is selected from heparin or heparin (LMW).

In some embodiments, the additional therapeutic agent comprises a vasodilator. In some instances, the vasodilator comprises angiotensin 1-7 or dipyridamole.

In some embodiments, the additional therapeutic agent comprises a lipoic acid, azithromycin, formoterol, or levamisole.

In some embodiments, the additional therapeutic agent comprises an mRNA-based COVID-19 vaccine. In some instances, the mRNA-based COVID-19 vaccine is BNT162 (BioNTech). In some instances, the mRNA-based vaccine is mRNA-1273, a lipid nanoparticle (LNP)-encapsulated mRNA vaccine which encodes a perfusion stabilized form of the Spike (S) protein from Moderna.

In some embodiments, the additional therapeutic agent comprises an siRNA-based COVID-19 vaccine. In some instances, the siRNA-based vaccine is a vaccine from Vir Biotechnology and Alnylam.

In some embodiments, the additional therapeutic agent comprises tocilizumab in combination with favipiravir.

In some embodiments, the additional therapeutic agent comprises leronlimab (PRO 140), a humanized IgG4 monoclonal antibody from CytoDyn.

In some embodiments, the additional therapeutic agent comprises ruxolitinib, a JAK1/JAK2 inhibitor from Incyte and Novartis.

In some embodiments, the additional therapeutic agent comprises INO-4800, a DNA vaccine from Inovio Pharmaceuticals and Beijing Advaccine Biotechnology.

In some embodiments, the additional therapeutic agent comprises eculizumab from Alexion Pharmaceuticals.

In some embodiments, the additional therapeutic agent comprises APNO1, a recombinant human angiotensin-converting enzyme 2 (rhACE2), from APEIRON Biologics.

In some embodiments, the additional therapeutic agent comprises danoprevir, an oral hepatitis C virus protease inhibitor, from Ascletis Pharma.

In some embodiments, the additional therapeutic agent comprises TJM2 (TJ003234), a neutralizing antibody against human granulocyte-macrophage colony stimulating factor (GM-CSF) from I-Mab.

In some embodiments, the additional therapeutic agent comprises selinexor, an oral selective inhibitor of nuclear export, from Karyopharm Therapeutics.

In some embodiments, the additional therapy treatment comprises stem cell therapy. In some instances, a plurality of stem cells are administered to the subject. In some cases, the additional therapy treatment comprises mesenchymal stem cell (MSC) therapy. In some cases, a plurality of MSCs are administered to the subject.

In some embodiments, the subject has an underlying disease or condition and the additional therapy comprises one or more treatments for treating the underlying disease or condition. In some instances, the underlying disease or condition comprises hypertension, cardiovascular disease, diabetes, or chronic lung disease. In some cases, the additional therapy comprises one or more treatments for treating hypertension, cardiovascular disease, diabetes, or chronic lung disease.

In some embodiments, the subject has a secondary infection and the additional therapy comprises one or more treatments for treating the secondary infection.

In some instances, the composition, the solution, or the soft gelatin capsule and the additional therapeutic agent are administered to the subject simultaneously.

In other instances, the composition, the solution, or the soft gelatin capsule and the additional therapeutic agent are administered to the subject sequentially. In some cases, the composition, the solution, or the soft gelatin capsule is administered to the subject prior to administering the additional therapeutic agent. In some cases, the composition, the solution, or the soft gelatin capsule is administered to the subject after administering the additional therapeutic agent.

In some cases, the subject is fasted prior to administration of the composition, the solution, or the soft gelatin capsule.

In some embodiments, the method further comprises administering to the subject an additional treatment regimen.

In some embodiments, the additional treatment regimen comprises a blood purification therapy, optionally an artificial-liver blood-purification system.

In some embodiments, the composition and the additional treatment regimen are administered to the subject either simultaneously or sequentially.

In some cases, the subject is a human.

### Dosing Regimens

In certain embodiments, disclosed herein is a dosing regimen comprising administering to a subject a first daily dose of from about 15 mg to about 1500 mg of 17-alpha hydroxyprogesterone caproate (17-HPC) on day 1 of a cycle. In some instances, the first daily dose comprises from about 15 mg to about 1000 mg, from about 15 mg to about 740 mg, from about 15 mg to about 720 mg, from about 15 mg to about 360 mg, from about 15 mg to about 240 mg, from about 15 mg to about 120 mg, from about 30 mg to about 1500 mg, from about 30 mg to about 1000 mg, from about 30 mg to about 740 mg, from about 30 mg to about 720 mg, from about 30 mg to about 360 mg, from about 30 mg to about 240 mg, from about 30 mg to about 120 mg, from about 120 mg to about 1500 mg, from about 120 mg to about 1000 mg, from about 120 mg to about 740 mg, from about 120 mg to about 720 mg, from about 120 mg to about 360 mg, from about 240 mg to about 1500 mg, from about 240 mg to about 1000 mg, from about 240 mg to about 740 mg, from about 240 mg to about 720 mg, from about 240 mg to about 360 mg, from about 360 mg to about 1500 mg, from about 360 mg to about 1000 mg, from about 360 mg to about 740 mg, or from about 360 mg to about 720 mg of 17-HPC. In some instances, the first daily dose comprises 120 mg of 17-HPC. In some instances, the first daily dose comprises 360 mg of 17-HPC. In some instances, the first daily dose comprises 720 mg of 17-HPC.

In some instances, the first daily dose comprises from about 120 mg to about 1500 mg, from about 120 mg to about 1000 mg, from about 120 mg to about 360 mg, from about 240 mg to about 720 mg, from about 240 mg to about 1500 mg, from about 240 mg to about 1000 mg, from about 240 mg to about 720 mg, from about 240 mg to about 360 mg, from about 360 mg to about 1500 mg, from about 360 mg to about 1000 mg, or from about 360 mg to about 720 mg of 17-HPC. In some instances, the first daily dose comprises 120 mg of 17-HPC. In some instances, the first daily dose comprises 360 mg of 17-HPC. In some instances, the first daily dose comprises 720 mg of 17-HPC. In some cases, the first daily dose of from about 120 mg to about 1500 mg is administered to a human subject. In some cases, the first daily dose of from about 120 mg to about 1500 mg is administered to a human subject 18 years of age or older. In some cases, the first daily dose of from about 120 mg to about 1500 mg is administered to a subject equivalent in age to a human subject 18 years of age or older.

In some instances, the first daily dose comprises from about 15 mg to about 740 mg, from about 15 mg to about 720 mg, from about 15 mg to about 360 mg, from about 15 mg to about 240 mg, from about 15 mg to about 120 mg, from about 15 mg to about 100 mg, from about 30 mg to about 740 mg, from about 30 mg to about 720 mg, from about 30 mg to about 360 mg, from about 30 mg to about 240 mg, from about 30 mg to about 120 mg, from about 30 mg to about 100 mg, from about 60 mg to about 740 mg, from about 60 mg to about 720 mg, from about 60 mg to about 360 mg, from about 60 mg to about 240 mg, from about 60 mg to about 120 mg, from about 120 mg to about 740 mg, from about 120 mg to about 720 mg, from about 120 mg to about 360 mg, from about 120 mg to about 240 mg, from about 240 mg to about 740 mg, from about 240 mg to about 720 mg, from about 240 mg to about 360 mg, from about 30 m6g to about 740 mg, or from about 360 mg to about 720 mg of 17-HPC. In some instances, the first daily dose comprises 15 mg of 17-HPC. In some instances, the first daily dose comprises 30 mg of 17-HPC. In some instances, the first daily dose comprises 120 mg of 17-HPC. In some instances, the first daily dose comprises 240 mg of 17-HPC. In some instances, the first daily dose comprises 360 mg of 17-HPC. In some instances, the first daily dose comprises 720 mg of 17-HPC. In some cases, the first daily dose of from about 15 mg to about 740 mg is administered to a human subject. In some cases, the first daily dose of from about 15 mg to about 740 mg is administered to a human subject 17 years of age or younger. In some cases, the first daily dose of from about 15 mg to about 740 mg is administered to a subject equivalent in age to a human subject 17 years of age or younger.

In some embodiments, the dosing regimen further comprises one or more additional daily doses of 17-HPC. In some cases, each of the one or more additional daily doses of 17-HPC comprises a range of from about 15 mg to about 1500 mg, from about 15 mg to about 1000 mg, from about 15 mg to about 740 mg, from about 15 mg to about 720 mg, from about 15 mg to about 360 mg, from about 15 mg to about 240 mg, from about 15 mg to about 120 mg, from about 30 mg to about 1500 mg, from about 30 mg to about 1000 mg, from about 30 mg to about 740 mg, from about 30 mg to about 720 mg, from about 30 mg to about 360 mg, from about 30 mg to about 240 mg, from about 30 mg to about 120 mg, from about 120 mg to about 1500 mg, from about 120 mg to about 1000 mg, from about 120 mg to about 740 mg, from about 120 mg to about 720 mg, from about 120 mg to about 360 mg, from about 240 mg to about 1500 mg, from about 240 mg to about 1000 mg, from about 240 mg to about 740 mg, from about 240 mg to about 720 mg, from about 240 mg to about 360 mg, from about 360 mg to about 1500 mg, from about 360 mg to about 1000 mg, from about 360 mg to about 740 mg, or from about 360 mg to about 720 mg of 17-HPC. In some cases, each of the one or more additional daily doses of 17-HPC comprises a range of from about 120 mg to about 1500 mg, from about 120 mg to about 1000 mg, from about 120 mg to about 360 mg, from about 240 mg to about 720 mg, from about 240 mg to about 1500 mg, from about 240 mg to about 1000 mg, from about 240 mg to about 720 mg, from about 240 mg to about 360 mg, from about 360 mg to about 1500 mg, from about 360 mg to about 1000 mg, or from about 360 mg to about 720 mg of 17-HPC. In some cases, each of the one or more additional daily doses of 17-HPC comprises a range of from about 15 mg to about 740 mg, from about 15 mg to about 720 mg, from about 15 mg to about 360 mg, from about 15 mg to about 240 mg, from about 15 mg to about 120 mg, from about 15 mg to about 100 mg, from about 30 mg to about 740 mg, from about 30 mg to about 720 mg, from about 30 mg to about 360 mg, from about 30 mg to about 240 mg, from about 30 mg to about 120 mg, from about 30 mg to about 100 mg, from about 60 mg to about 740 mg, from about 60 mg to about 720 mg, from about 60 mg to about 360 mg, from about 60 mg to about 240 mg, from about 60 mg to about 120 mg, from about 120 mg to about 740 mg, from about 120 mg to about 720 mg, from about 120 mg to about 360 mg, from about 120 mg to about 240 mg, from about 240 mg to about 740 mg, from about 240 mg to about 720 mg, from about 240 mg to about 360 mg, from about 30 m6g to about 740 mg, or from about 360 mg to about 720 mg of 17-HPC. In some cases, each of the one or more additional daily doses of 17-HPC comprises a range of from about 120 mg to about 1500 mg, from about 120 mg to about 1000 mg, from about 120 mg to about 720 mg, from about 120 mg to about 360 mg, from about 240 mg to about 720 mg, from about 240 mg to about 360 mg, or from about 360 mg to about 720 mg of 17-HPC. In some instances, each of the one or more additional daily doses comprises 120 mg of 17-HPC. In some instances, each of the one or more additional daily doses comprises 360 mg of 17-HPC. In some instances, each of the one or more additional daily doses comprises 720 mg of 17-HPC.

In some embodiments, the dosing regimen comprises a first daily dose of from about 6 mg/kg to about 12 mg/kg of 17-HPC. In some instances, the first daily dose is about 6 mg/kg of 17-HPC. In some instances, the first daily dose is about 12 mg/kg of 17-HPC.

In some embodiments, the dosing regimen further comprises one or more additional daily doses of 17-HPC, in which the one or more additional daily doses of 17-HPC is from about 6 mg/kg to about 12 mg/kg. In some cases, the one or more additional daily dose of 17-HPC is about 6 mg/kg. In some cases, the one or more additional daily dose of 17-HPC is about 12 mg/kg.

In some embodiments, the cycle of the dosing regimen is from about 7 to about 30 days, optionally a 7, 14, 21, 28, or 30 day cycle. In some instances, the cycle of the dosing regimen is about 7 days. In some instances, the cycle of the dosing regimen is about 14 days. In some instances, the cycle of the dosing regimen is about 21 days. In some instances, the cycle of the dosing regimen is about 28 days. In some instances, the cycle of the dosing regimen is about 30 days.

In some embodiments, the cycle of the dosing regimen is a 7-day cycle and the regimen comprises a first daily dose administered on day 1 and a second daily dose administered on a day selected from day 5 to day 7 of the cycle. In some cases, the second daily dose is administered on day 5. In some cases, the second daily dose is administered on day 6. In some cases, the second daily dose is administered on day 7. In some cases, the first daily dose and the second daily dose each independently comprises from about 120 mg to about 720 mg, from about 120 mg to about 360 mg, from about 240 mg to about 720 mg, from about 240 mg to about 360 mg, or from about 360 mg to about 720 mg of 17-HPC. In some cases, the first daily dose comprises 360 mg or 720 mg of 17-HPC. In some cases, the first daily dose comprises 360 mg of 17-HPC. In some cases, the first daily dose comprises 720 mg of 17-HPC. In some cases, the second daily dose comprises 360 mg or 720 mg of 17-HPC. In some cases, the second daily dose comprises 360 mg of 17-HPC. In some cases, the second daily dose comprises 720 mg of 17-HPC.

In some embodiments, the cycle of the dosing regimen is a 7-day cycle and the regimen comprises a daily dose of from about 120 mg to about 720 mg of 17-HPC administered to a subject for about 5 days in the cycle. In some instances, the daily dose is administered to the subject from day 1 to day 5 of the cycle. In some instances, each of the daily dose comprises from about 120 mg to about 720 mg, from about 120 mg to about 360 mg, from about 240 mg to about 720 mg, from about 240 mg to about 360 mg, or from about 360 mg to about 720 mg of 17-HPC. In some cases, each of the daily dose comprises 360 mg of 17-HPC. In some cases, each of the daily dose comprises 720 mg of 17-HPC.

In some embodiments, the cycle of the dosing regimen is a 7-day cycle and the regimen comprises a daily dose of from about 120 mg to about 720 mg of 17-HPC administered to a subject for about 6 days in the cycle. In some instances, the daily dose is administered to the subject from day 1 to day 6 of the cycle. In some instances, each of the daily dose comprises from about 120 mg to about 720 mg, from about 120 mg to about 360 mg, from about 240 mg to about 720 mg, from about 240 mg to about 360 mg, or from about 360 mg to about 720 mg of 17-HPC. In some cases, each of the daily dose comprises 360 mg of 17-HPC. In some cases, each of the daily dose comprises 720 mg of 17-HPC.

In some embodiments, the cycle of the dosing regimen is a 7-day cycle and the regimen comprises administering to a subject from about 120 mg to about 720 mg of 17-HPC every 12 hours starting at day 1 for about 5 to about 6 days in the cycle. In some instances, the regimen comprises administering to the subject about 120 mg of 17-HPC every 12 hours. In some instances, the regimen comprises administering to the subject about 360 mg of 17-HPC every 12 hours. In some instances, the regimen comprises administering to the subject about 720 mg of 17-HPC every 12 hours. In some cases, the regimen comprises administration of 17-HPC every 12 hours for about 5 days in the cycle. In some cases, the regimen comprises administration of 17-HPC every 12 hours for about 5.5 days in the cycle. In some cases, the regimen comprises administration of 17-HPC every 12 hours for about 6 days in the cycle.

In some embodiments, the dosing regimen further comprises administering to the subject an additional therapeutic agent. In some instances, the additional therapeutic agent is a glucocorticoid. In some cases, the additional therapeutic agent is selected from hydrocortisone, cortisone acetate, dexamethasone, prednisone, prednisolone, methylprednisolone, betamethasone, triamcinolone, beclometasone, Paramethasone, fluticasone, fludrocortisone acetate, deoxycorticosterone acetate, Fluprednisolone, fluticasone propionate, budesonide, beclomethasone dipropionate, flunisolide, triamcinolone acetonide, or a combination thereof. In some cases, the additional therapeutic agent is dexamethasone (DEX). In some cases, dexamethasone is administered to the subject at a dose of about 3 mg or higher per day. In some instances, dexamethasone is administered to the subject at a dose of about 6 mg per day. In some cases, the additional therapeutic agent is budesonide (BUD). In some cases, the additional therapeutic agent is prednisone.

### Kits and Articles of Manufacture

In certain embodiments, disclosed herein is a kit or article of manufacture comprising a composition comprising 17-HPC or a soft gelatin capsule comprising a liquid filling comprising 17-HPC.

In some embodiments, the kit or article of manufacture further comprises a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

A kit typically includes labels listing contents and/or instructions for use, and package inserts with instructions for use. A set of instructions will also typically be included.

### EXAMPLES

These examples are provided for illustrative purposes only and not to limit the scope of the claims provided herein.

### Example 1—Pharmacodynamic Study of 17-HPC in Smoke Exposure-Induced COPD Rats

To examine the in vitro and in vivo efficacy of 17-HPC inhalation in chronic obstructive pulmonary disease (COPD) rats, a rat model of COPD was established by exposing Wistar rats to CS for 1 hour every day for a total of 180 days. Seventy-two Wistar rats were randomly divided into the blank control group, model group, 17-HPC (0.5 mg/ml, 0.25 mg/ml and 0.1 mg/ml) groups, BUD (0.5 mg/ml and 0.1 mg/ml) groups, and combination treatment (0.5 mg/ml BUD+0.25 mg/ml 17-HPC and 0.1 mg/ml BUD+0.25 mg/ml 17-HPC) groups. Drug concentration is the nebulized solution concentration. Lung function (forced vital capacity) of rats was measured using the small animal spirometer, and differential count in the BALF was determined under the microscope. Serum and BALF cytokine (IL-6, IL-17, TNF-α and IL-β) levels were measured by ELISA. Inflammatory cell infiltration area and MLI in rat lung tissues were determined by HE staining.

Lung function of rats was measured using the small animal spirometer after 24 weeks of CS exposure. FEV0.2/FVC (200 ms) was lower in CS-exposed rats (67.4+4.20%) than in normal rats (79.2+1.79%) (p<0.01), indicating that the model was successfully constructed. Compared with the model group, 17-HPC improved FEV0.2/FVC % in a dose-dependent manner. FEV0.2/FVC % was higher in the BUD group than in the model group. FEV0.2/FVC % was also higher in the 17-HPC+BUD group than in the model group, but efficacy was not superior to that of 17-HPC or BUD alone.

Long-term regular CS exposure resulted in increased number of inflammatory cells in rat BALF. In contrast, 17-HPC, BUD, and 17-HPC+BUD all reduced the percentage of neutrophils in rat BALF BUD was superior to 17-HPC in reducing the proportion of BALF neutrophils at the equivalent doses. The efficacy of high dose BUD was equivalent to that of high dose BUD+17-HPC. However, the efficacy of low dose BUD+17-HPC was superior to those of low dose BUD alone or 17-HPC alone.

Aside from the low dose (0.1 mg/ml) 17-HPC group, different doses of 17-HPC and BUD can downregulate TNF-α, IL-1β, IL-6 and IL-17 levels in the BALF. Serum TNF-α, IL-1β, IL-6 and IL-17 levels were also downregulated in the treatment groups in a dose-dependent manner.

HE staining of lung tissue sections showed inflammatory cell infiltration in model rats, which was attenuated in the 17-HPC groups. High dose 17-HPC can antagonize inflammatory cell infiltration in the lung tissues of COPD rats, and the pathological characteristics were similar to those of normal rats. The efficacy of 17-HPC was similar to those of BUD and combination treatment at the equivalent doses. Aside from the 0.1 mg/ml 17-HPC group, MLI was decreased in the other 17-HPC groups and BUD groups in a dose-dependent manner. The efficacy of high dose BUD was equivalent to that of high dose BUD+17-HPC. However, the efficacy of low dose BUD+17-HPC was superior to that of low dose BUD alone or 17-HPC alone.

At 24 weeks, the weight of control animals continued to increase whereas 5 mg/ml BUD inhibited weight increase and was lower than in the model group. Inhibition of weight increase was lower in 17-HPC group than in the same dose BUD group. There was no difference in weight increase between the 17-HPC groups and the model group. Weight changes in the combination treatment groups were similar to those in BUD alone groups.

### Example 2—Seven-Day Inhalation Toxicity Study in Rats

A 7-day inhalation toxicity study in rats was conducted under GLP conditions. In this study, aerosolized 17-HPC was administered to Sprague-Dawley rats (10/sex/group) at 8, 24, and 72 mg/kg/day for 7 consecutive days. A control group of rats was dosed with aerosolized vehicle. Animals were observed daily for clinical signs. Body weight and food consumption were recorded at pretest, and then on Days 1, 4, and 7. In order to evaluate reversibility of 17-HPC treatment, a satellite rat group (5/sex/group) was dosed by inhalation at 72 mg/kg/day for 7 days, and then was sacrificed on Day 14. Except for the rats in the satellite group, all animals were sacrificed on Day 8. Hematology and biochemistry examinations were performed at the end of the study. Organ weight, gross pathology, and histopathology evaluations were performed.

Preliminary results of this experiment showed that no clinical signs and body weight change were observed at the 8 mg/kg/day group. Hematological and biochemical parameters were comparable between the animals treated with vehicle and 17-HPC at 8 mg/kg/day. Significant changes in organ weights, necropsy and histopathology were not recorded. After a 7-day recovery period, there were no remarkable pathological alterations in any animals from the satellite group. Histopathological examinations on the animal tissues from the 24 and 72 mg/kg/day groups have not been completed prior to submission of this pre-IND package.

Based on the preliminary results of this study, inhalational administration of 17-HPC at 8 mg/kg/day (48 mg/m²/day) was determined as the no observed adverse effect level (NOAEL). The proposed clinical study is a single dose inhalation study starting at a dose level of 300 μg, which is approximately 6 μg/kg/day (222 μg/m²/day) based on a human body weight of 50 kg/person. Therefore, the NOAEL generated in this inhalation study is approximately 216 times greater than the proposed initial clinical dose based on body surface areas.

### Example 3—PK Analysis Report for Oral Formulations of PR2005 in Female Dogs

This study determined the pharmacokinetic parameters of the test drug PR2005 (also referred to herein as 17-HPC) in different formulations after oral gavage or capsule administrations in dogs. This study further determined the relative bioavailability of the test drug PR2005 in each formulation, compared to intramuscular injection solution (MAKENA®).

### Method of PK Analysis:

PK parameters were estimated, including maximum plasma drug concentration (Cₘₐₓ), time to reach Cₘₐₓ (Tₘₐₓ), time delay in absorption process (T_{lag}), area under the plasma concentration-time curve from time 0 to the last quantifiable data point (AUCₗₐₛₜ), area under the plasma concentration-time curve from time zero to infinity (AUC_{inf}), plasma half-life (t_{1/2}) and apparent plasma clearance (CL/F), apparent volume of distribution (Vz/F). All these parameters were computed using standard non-compartmental methods of analysis. Geometric mean and CV % are reported for most of PK parameters, and median with range is reported for Tₘₐₓ and T_{lag}. Plasma PR2005 PK data was analyzed in Phoenix WinNonLin 8.1 (Certara USA, Inc.).

Log-transformed values of AUC_{inf} were analyzed using a linear mixed-effects model. Least squares mean (LSM) for each treatment group and 90% confidence interval (CI) for the mean of the pairwise differences were estimated using this model. After back transformation from the log scale, estimates of geometric LSM and 90% CI for the ratio of means were calculated. Relative bioavailability (Fr %) was calculated as the ratio of dose normalized geometric least square mean (LSM) for test formulation and IM solution:

$$Fr \% = \frac{{AUC}_{test} / {Dose}_{test}}{{AUC}_{ref} / {Dose}_{ref}} \times 100 \%$$

### Formulations:

PR2005 250 mg intramuscular injection: 250 mg 17-HPC, 28.6% v/v of castor oil (CAS 8001-79-4), 46% v/v of benzyl benzoate (CAS 120-51-4), and 2% v/v of benzyl alcohol.

PR2005 250 mg oral (powder in capsules): 20% w/w of 17-HPC and 80% w/w mannitol. The particle size of 17-HPC is from about 2 to about 10 μm.

PR2005 250 mg Suspension #1: 30% w/w of 17-HPC and 70% castor oil (CAS 8001-79-4). The particle size of 17-HPC is from about 5 to about 30 μm.

PR2005 250 mg and 750 mg Solution #3: 30% w/w of 17-HPC, 63% w/w of benzyl benzoate (CAS 120-51-4), and 7% w/w of macrogolglycerol ricinoleate (CAS 61791-12-6).

PR2005 250 mg Solution #5: 30% w/w of 17-HPC, 63% w/w of benzyl benzoate (CAS 120-51-4), and 7% w/w of polysorbate 80 (CAS 9005-65-6).

Table 18 illustrates the details of the study.

TABLE 18

| Trial | Group | Route | Test Item | Dose^{a} (mg) | No. of Animals |
|---|---|---|---|---|---|
| 1 | 1F | OG | Solution #3 (3x) | 750 | 3 |
| | 2F | OG | Solution #3 | 250 | 2 |
| | 3F | OG | Solution #5 | 250 | 2 |
| | 4F | OG | Ready-made Suspension S1 | 250 | 2 |
| | 5F | OG | Powder for Aqueous Suspension | 250 | 2 |
| 2 | 1F | IM | MAKENA ® (reference) | 250 | 3 |
| | 2F | OC | Powder for Aqueous Suspension^{b} | 250 | 2 |
| | 3F | OC | Solution #3 | 250 | 2 |
| | 4F | OC | Solution #5 | 250 | 2 |
| | 5F | OC | Ready-made Suspension S1 | 250 | 2 |
| 3 | 2F | OC | Ready-made Suspension S1 | 250 | 2 |
| | 3F | OC | Powder for Aqueous Suspension^{b} | 250 | 2 |
| | 4F | OC | Solution #3 | 250 | 2 |
| | 5F | OC | Solution #5 | 250 | 2 |
| 4 | 2F | OC | Solution #5 | 250 | 2 |
| | 3F | OC | Ready-made Suspension S1 | 250 | 2 |
| | 4F | OC | Powder for Aqueous Suspension^{b} | 250 | 2 |
| | 5F | OC | Solution #3 | 250 | 2 |

^{a}Doses represent active ingredient 17-HPC.
^{b}In trials 2-4, the test item “Powder for Aqueous Suspension” was not formulated to be a suspension, and is instead given as a pure powder, filled into capsules.

OG is oral gavage; OC is capsule

The test items were administered on the first day of each trial. Each animal group was rotated through each trial with at least 4 days between trials and a maximum 7-day washout between each test item administration.

### Results:

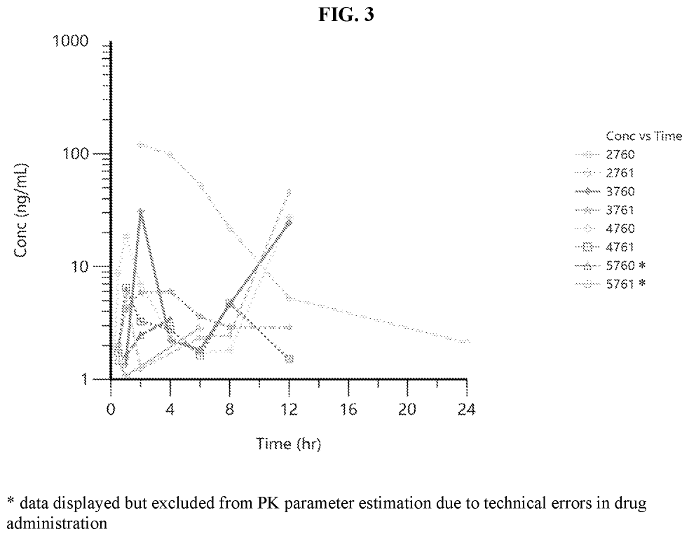

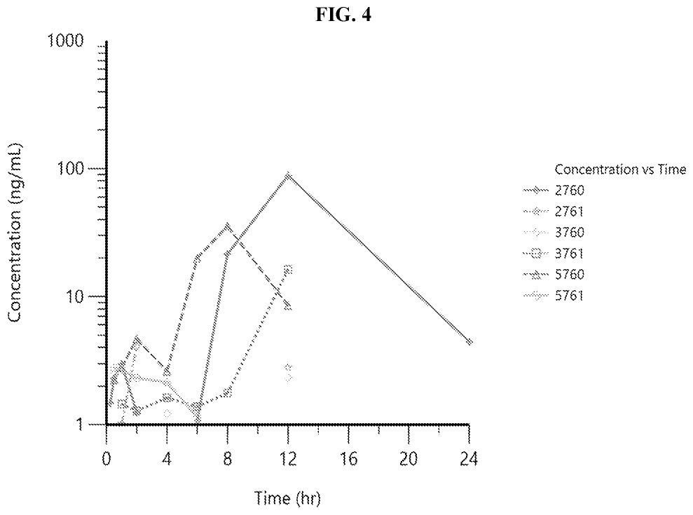

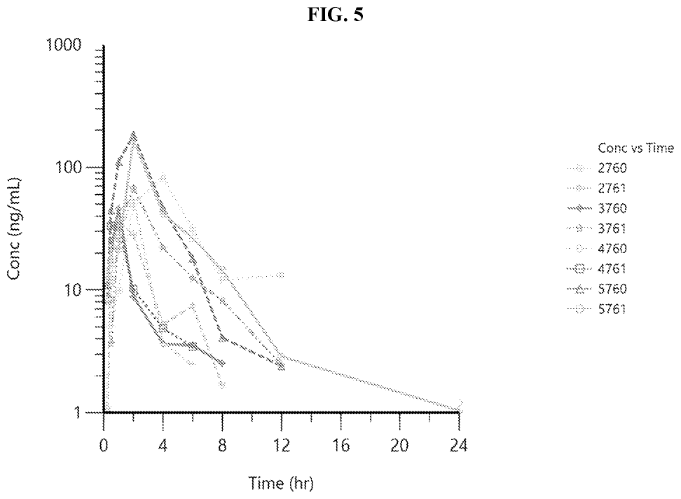

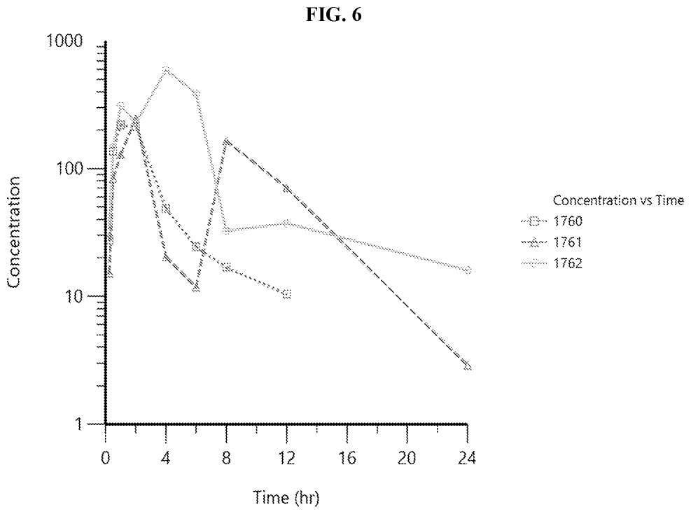

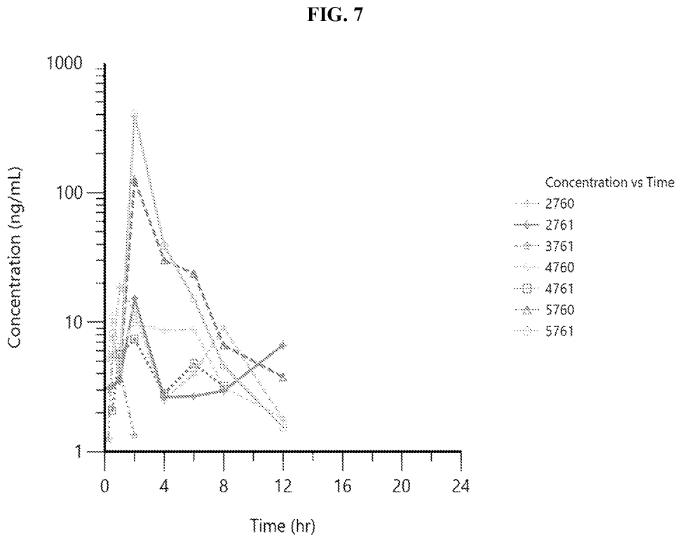

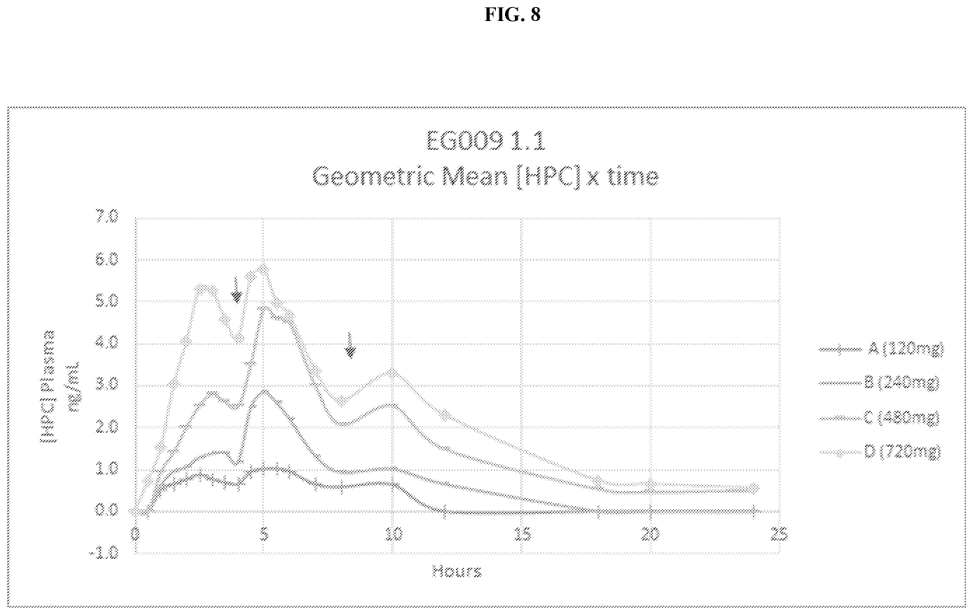

PR2005 (hydroxyprogesterone) plasma concentration-time profiles following IM and oral administration of various formulations are shown in FIG. 2 to FIG. 6. The PK parameter estimates following administration of PR2005 in each formulation are summarized in Tables 4 to 11.

Following single 250 mg IM infusion to dogs, PR2005 was quantifiable in plasma at all planned sampling points (up to the last collected time point of 168 hours) (FIG. 2). As indicated in Table 4, Tmax occurred at 48 hours post dose in all three dogs. Half-life (t_{1/2}) was 48.38 hours. Apparent plasma clearance (CL/F, 35.68 L/hr) was low to moderate, and apparent distribution volume (Vz/F, 2485L) was large. Very low variability (<10%) was observed in most PK parameters, including Cₘₐₓ and AUC_{inf}. These PK characteristics of hydroxyprogesterone were consistent with those reported in humans following IM infusion.

Following single 250 mg PR2005 oral formulations of powder in capsules, Suspension #1, and Solution #5, large variability was observed in the PK profiles and in most of PK parameter estimates. The absorption following oral administration of these formulations appeared inconsistent among animals receiving the same oral formulation. Tₘₐₓ ranged from 0.5 to 12 hours post dose. The terminal half-life (t_{1/2}<5 hours) was short, compared to the one following IM infusion. The geometric LSM of relative bioavailability was less than 2% for all three oral formulations, compared to IM infusion solution.

Following a single 250 mg PR2005 oral formulation of Solution #3, moderate variability was observed in the PK profiles and in most of PK parameter estimates. Geometric mean of Cₘₐₓ was 69.21 μg/L, which was comparable to the one after 250 mg IM infusion. Tₘₐₓ occurred from 0.5 to 4 hours. T_{lag} ranged from 0 to 0.25, indicating very limited delay in absorption after administration. The geometric LSM of relative bioavailability was estimated approximately 3% for Solution #3 containing 250 mg PR2005, compared to IM infusion solution.

Following a single 750 mg PR2005 oral formulation of Solution #3, moderate to low variability was observed in the PK profiles and in most of PK parameter estimates. Tₘₐₓ occurred from 1 to 4 hours. No absorption delay was observed in dogs (T_{lag}=0). Geometric mean of Cₘₐₓ and AUC_{inf} were 318.04 μg/L and 1668.24 μg·hr/L, which were greater than dose proportional, compared to those following 250 mg PR2005 administration of the same formulation. Geometric mean of apparent CL/F was 449.58 L/hr, which was smaller than the one after 250 mg PR2005 administration with the same formulation. This result suggested that nonlinearity may occur in absorption and/or elimination process following oral administration of PR2005 in Solution #3. However, this finding was inconclusive due to the observed PK variability with oral administration of Solution #3. Literature studies indicate that hydroxyprogesterone is a substrate of ABCB1 efflux transporters (e.g. Bloise, E., et al., “ATP-binding cassette transporters in reproduction: a new frontier,” Hum Repro Update, 22(2): 164-181, 2016). Thus, the saturation of efflux transporters in intestines and/or livers may contributed to the complexity of hydroxyprogesterone PK following oral administration of PR2005. The geometric LSM of relative bioavailability for Solution #3 containing 750 mg was 7.92%, compared to IM solution. This value was greater than the one for Solution #3 containing 250 mg, but was not statistically significant.

The relative bioavailabilities for 250 mg PR2005 oral formulations of powder in capsules, Suspension #1, and Solution #5 were less than 2%, compared to IM infusion solution. The relative bioavailability for Solution #3 was higher than the other three tested formulations.

TABLE 4
Plasma PR2005 PK parameter estimates following
250 mg intramuscular injection to dogs

| | | | | Geometric | Geometric |
|---|---|---|---|---|---|
| ID | IM1760 | IM1761 | IM1762 | Mean | CV % |
| Dose (mg) | 250 | 250 | 250 | | |
| Cmax (μg/L) | 62.4 | 55.9 | 56.0 | 58.1 | 6.41 |
| Tmax (hr)^{§} | 48 | 48 | 48 | 48 | n/a |
| AUC_{inf} (μg · hr/L) | 7455.49 | 6523.72 | 7105.66 | 7017.65 | 6.67 |
| AUCₗₐₛₜ (μg · hr/L) | 6342.45 | 5583.82 | 6047.08 | 5991.12 | 6.38 |
| Cl/F (L/hr) | 33.53 | 38.32 | 35.18 | 35.68 | 6.82 |
| V (L) | 2646.99 | 2668.04 | 2140.48 | 2485.17 | 12.0 |
| t_{1/2} (hr) | 54.72 | 48.26 | 42.17 | 48.38 | 13.0 |

^{§}Expressed as median and range

TABLE 5
Plasma PR2005 PK parameter estimates following 250
mg oral administration to dogs (Powder in capsules)

| | | | | | | | Geometric | Geometric |
|---|---|---|---|---|---|---|---|---|
| ID | D2760 | D2761 | D3760 | D3761 | D4760 | D4761 | Mean | CV % |
| Dose | 250 | 250 | 250 | 250 | 250 | 250 | | |
| (mg) | | | | | | | | |
| Cmax | 27.3 | 45.7 | 31.2 | 6.01 | 122 | 6.47 | 23.86 | 169.71 |
| (μg/L) | | | | | | | | |
| Tmax | 12 | 12 | 2 | 4 | 2 | 1 | 3 | 1-12 |
| (hr)^{§} | | | | | | | | |
| T-lag | 0 | 0.5 | 0.5 | 0 | 0 | 0.25 | 0.13 | 0-0.5 |
| (hr)^{§} | | | | | | | | |
| AUC_{inf} | 121.06 | 154.76 | 130.45 | 49.82 | 673.24 | 40.37 | 122.07 | 131.17 |
| (μg · hr/L) | | | | | | | | |
| AUCₗₐₛₜ | 121.06 | 154.76 | 130.45 | 49.81 | 645.63 | 35.03 | 118.39 | 135.07 |
| (μg · hr/L) | | | | | | | | |
| CL/F | 2065.02 | 1615.42 | 1916.41 | 5018.57 | 371.34 | 6193.19 | 2047.99 | 131.17 |
| (L/hr) | | | | | | | | |
| V (L) | 1979.69 | 1487.44 | 1854.47 | 5865.02 | 4858.15 | 21741.27 | 3874.48 | 133.33 |
| t_{1/2} (hr) | 0.66 | 0.64 | 0.67 | 0.81 | 9.07 | 2.43 | 1.31 | 147.63 |

^{§}Expressed as median and range

TABLE 6
Plasma PR2005 PK parameter estimates following 250
mg oral administration to dogs (Suspension #1)

| | | | | | | | Geometric | Geometric |
|---|---|---|---|---|---|---|---|---|
| ID | D2760 | D2761 | D3760 | D3761 | D5760 | D5761 | Mean | CV % |
| Dose | 250 | 250 | 250 | 250 | 250 | 250 | | |
| (mg) | | | | | | | | |
| Cmax | 87.80 | 4.08 | 2.48 | 16.30 | 35.60 | 2.78 | 10.62 | 281.24 |
| (μg/L) | | | | | | | | |
| Tmax | 12 | 2 | 0.5 | 12 | 8 | 0.5 | 5 | 0.5-12 |
| (hr)^{§} | | | | | | | | |
| T-lag | 0 | 0.5 | 0.25 | 0.5 | 0.25 | 0.25 | 0.25 | 0-0.5 |
| (hr)^{§} | | | | | | | | |
| AUC_{inf} | 818.57 | | | 144.68 | 202.79 | 15.69 | 139.32 | 369.06 |
| (μg · hr/L) | | | | | | | | |
| AUCₗₐₛₜ | 800.72 | 15.21 | 8.03 | 144.68 | 178.86 | 11.99 | 55.84 | 547.97 |
| (μg · hr/L) | | | | | | | | |
| CL/F | 305.41 | | | 1727.94 | 1232.81 | 15935.30 | 1794.39 | 369.06 |
| (L/hr) | | | | | | | | |
| V (L) | 1228.03 | | | 1727.73 | 3454.26 | 51317.78 | 4403.79 | 406.82 |
| t_{1/2} (hr) | 2.79 | | | 0.69 | 1.94 | 2.23 | 1.70 | 68.04 |

^{§}Expressed as median and range

Dog #4760 and Dog #4761 were excluded since all concentrations are BLQ. Lower limit of qualification (LLOQ) is 1.00 ng/mL.

Table 7A and Table 7B. Plasma PR2005 PK parameter estimates following 250 mg oral administration to dogs (Solution #3)

TABLE 7A

| ID | D2760 | D2761 | D3760 | D3761 | D4760 | D4761 |
|---|---|---|---|---|---|---|
| Dose (mg) | 250 | 250 | 250 | 250 | 250 | 250 |
| Cmax (mg/L) | 82.90 | 35.40 | 46.40 | 67.70 | 55.80 | 33.30 |
| Tmax (hr)^{§} | 4.00 | 1.00 | 1.00 | 2.00 | 2.00 | 0.50 |
| T-lag (hr)^{§} | 0.00 | 0.00 | 0.25 | 0.25 | 0.00 | 0.00 |
| AUC_{inf} (mg · hr/L) | 485.64 | 109.08 | 88.19 | 234.48 | 161.92 | 87.91 |
| AUCₗₐₛₜ (mg · hr/L) | 376.27 | 103.12 | 73.00 | 226.50 | 130.68 | 67.89 |
| Cl/F (L/hr) | 514.78 | 2291.97 | 2834.74 | 1066.19 | 1543.99 | 2843.86 |
| V (L) | 4265.28 | 8074.57 | 17021.43 | 3503.01 | 39857.89 | 16499.54 |
| t_{1/2} (hr) | 5.74 | 2.44 | 4.16 | 2.28 | 17.89 | 4.02 |

^{§}Expressed as median and range

TABLE 7B

| | | | Geometric | Geometric |
|---|---|---|---|---|
| ID | D5760 | D5761 | Mean | CV % |
| Dose (mg) | 250 | 250 | | |
| Cmax (mg/L) | 184.00 | 167.00 | 69.21 | 72.49 |
| Tmax (hr)^{§} | 2.00 | 2.00 | 2 | 0.5-4^{§} |
| T-lag (hr)^{§} | 0.00 | 0.00 | 0 | 0-0.25^{§} |
| AUC_{inf} (mg · hr/L) | 542.11 | 499.39 | 212.90 | 93.12 |
| AUCₗₐₛₜ (mg · hr/L) | 524.02 | 487.10 | 186.33 | 100.65 |
| Cl/F (L/hr) | 461.16 | 500.61 | 1174.25 | 93.12 |
| V (L) | 3476.36 | 5917.97 | 8520.66 | 109.33 |
| t_{1/2} (hr) | 5.23 | 8.19 | 5.03 | 74.70 |

^{§}Expressed as median and range

TABLE 8
Plasma PR2005 PK parameter estimates following
750 mg oral administration to dogs (Solution #3)

| | | | | Geometric | Geometric |
|---|---|---|---|---|---|
| ID | D1760 | D1761 | D1762 | Mean | CV % |
| Dose (mg) | 750 | 750 | 750 | | |
| Cmax (μg/L) | 220.00 | 247.00 | 592.00 | 318.04 | 58.33 |
| Tmax (hr)^{§} | 1.00 | 2.00 | 4.00 | 2 | 1-4 |
| T-lag (hr)^{§} | 0 | 0 | 0 | 0 | 0 |
| AUC_{inf} (μg · hr/L) | 846.37 | 1652.14 | 3320.22 | 1668.24 | 77.16 |
| AUCₗₐₛₜ (μg · hr/L) | 759.63 | 1641.34 | 3093.38 | 1568.23 | 79.98 |
| Cl/F (L/hr) | 886.14 | 453.96 | 225.89 | 449.58 | 77.16 |
| V (L) | 7391.06 | 1701.24 | 3202.57 | 3427.60 | 84.91 |
| t_{1/2} (hr) | 5.78 | 2.60 | 9.83 | 5.28 | 75.25 |

^{§}Expressed as median and range

Table 9A and Table 9B. Plasma PR2005 PK parameter estimates following 250 mg oral administration to dogs (Solution #5)

TABLE 9A

| ID | D2760C | D2761C | D3761g | D4760C | D4761C |
|---|---|---|---|---|---|
| Dose (mg) | 250 | 250 | 250 | 250 | 250 |
| Cmax (μg/L) | 18.70 | 15.20 | 5.74 | 11.00 | 7.43 |
| Tmax (hr)^{§} | 1.00 | 2.00 | 0.50 | 0.50 | 2.00 |
| T-lag (hr)^{§} | 0.25 | 0.00 | 0.00 | 0.00 | 0.25 |
| AUC_{inf} | 82.44 | | 7.69 | 86.13 | 50.42 |
| (μg · hr/L) | | | | | |
| AUCₗₐₛₜ | 78.05 | 60.23 | 6.36 | 72.94 | 34.56 |
| (μg · hr/L) | | | | | |
| Cl/F (L/hr) | 3032.67 | | 32519.10 | 2902.51 | 4958.26 |
| V (L) | 7469.91 | | 32403.93 | 21042.79 | 24583.58 |
| t_{1/2} (hr) | 1.71 | | 0.69 | 5.03 | 3.44 |

^{§}Expressed as median and range

TABLE 9B

| | | | Geometric | Geometric |
|---|---|---|---|---|
| ID | D5760C | D5761C | Mean | CV % |
| Dose (mg) | 250 | 250 | | |
| Cmax (μg/L) | 126.00 | 406.00 | 25.40 | 335.24 |
| Tmax (hr)^{§} | 2.00 | 2.00 | 1.50 | 0.5-2 |
| T-lag (hr)^{§} | 0.50 | 0.25 | 0.25 | 0-0.5 |
| AUC_{inf} (μg · hr/L) | 353.71 | 745.99 | 94.81 | 342.78 |
| AUCₗₐₛₜ (μg · hr/L) | 326.87 | 740.37 | 78.42 | 308.93 |
| Cl/F (L/hr) | 706.80 | 335.13 | 2636.91 | 342.78 |
| V (L) | 5018.20 | 1222.59 | 9570.07 | 191.69 |
| t_{1/2} (hr) | 4.92 | 2.53 | 2.52 | 87.60 |

^{§}Expressed as median and range

TABLE 10
Relative bioavailability of different PR2005 formulations in dogs

| | Intramuscular | | | | | |
|---|---|---|---|---|---|---|
| | injection | Powder in | Suspension | Solution | Solution | Solution |
| | solution | capsules | #1 | #3 | #3 | #5 |
| Dose (mg) | 250 | 250 | 250 | 250 | 750 | 250 |
| ^{a}AUC_{inf} | 7017.65 | 122.07 | 139.32 | 211.99 | 1668.24 | 94.81 |
| (μg · hr/L) | | | | | | |
| ^{b}Fr (%) | n/a | 1.74 (0.42, | 1.99 (0.45, | 3.02 (0.78, | 7.92 (1.56, | 1.35 (0.33, |
| | | 7.13) | 8.79) | 11.65) | 40.37) | 5.53) |

^{a}Expressed as Geometric LSM;
^{b}Expressed as a ratio of dose normalized geometric LSMs (90% CI)

TABLE 11A
Blood Level: Mean and 99% CI of Cmax
of the different PR2005 formulations

| | | Cmax (μg/L) | | | |
|---|---|---|---|---|---|
| | | | | 2.576 × SD | 99% CI |
| Formulation | Dose | Mean | SD | (99% CI) | Upper limit |
| Intramuscular | 250 | 58.1 | 3.72422 | 9.5937 | 67.69 |
| injection | mg | | | | |
| solution | | | | | |
| Powder in | 250 | 39.78 | 43.06858 | 110.9447 | 163.39 |
| capsules | mg | | | | |
| Suspension | 250 | 24.84 | 33.37742 | 85.9802 | 110.82 |
| #1 | mg | | | | |
| Solution #5 | 250 | 84.30 | 148.18992 | 381.7372 | 466.03 |
| | mg | | | | |
| Solution #3 | 250 | 84.06 | 58.91558 | 151.2363 | 235.30 |
| | mg | | | | |
| Solution #3 | 750 | 353.00 | 207.41986 | 532.4468 | 885.45 |
| | mg | | | | |

TABLE 11B
Blood Level: Mean and 99% CI of AUC of the different PR2005
formulations

| | | AUC (μg · hr/L) | | | |
|---|---|---|---|---|---|
| | | | | 2.576 × SD | 99% CI |
| Formulation | Dose | Mean | SD | (99% CI) | Upper limit |
| Powder in | 250 | 194.95 | 238.7275584 | 685.1480925 | 880.0980925 |
| capsules | mg | | | | |
| Suspension | 250 | 193.2483333 | 306.7395623 | 880.3425439 | 1073.590877 |
| #1 | mg | | | | |
| Solution #5 | 250 | 188.4828571 | 265.3883205 | 761.6644798 | 950.147337 |
| | mg | | | | |
| Solution #3 | 250 | 276.08 | 199.2572062 | 511.4932484 | 787.5732484 |
| | mg | | | | |
| Solution #3 | 750 | 1939.58 | 1261.724349 | 3238.846405 | 5178.423071 |
| | mg | | | | |

99% CI refers to 99% Confidence Interval.

Studies have shown that 17-HPC as an oral formulation provides low bioavailability, even under large dose settings. To achieve a level of bioavailability that exerts a therapeutic effect, studies have shown that intramuscular injection is utilized. Unexpected, the inventors have shown in Tables 11A and 11B that Solution #3 (e.g., at the 750 mg dose), provides a higher level of bioavailability compared to the intramuscular injection and the other tested formulations. As illustrated in Table 11A, the blood level (Cmax, or the peak serum concentration of the drug achieved in the body after the drug has been administered) of Solution #3 at 750 mg dose is about 353 μg/L. This is higher than the blood levels (Cmax values) of the intramuscular injection (˜58.1 μg/L) and that of the other tested formulations. Similarly as shown in Table 11B, the AUC (or the actual body exposure to the drug after administration of a dose of the drug) for Solution #3 is also higher compared to that of the other tested formulations.

Table 19 illustrates the combined mean 17-HPC pharmacokinetic parameters in female dog plasma for both oral and capsule administrations.

TABLE 19

| | | Dose | | | | | | | |
|---|---|---|---|---|---|---|---|---|---|
| | | level | Cₘₐₓ | Tₘₐₓ | AUC₀₋ₜ | AUC_{0-inf} | t_{1/2} | Fᵣₑₗ^{a} | Fᵣₑₗ^{b} |
| Treatment | Route | (mg) | (ng/mL) | (h) | (h*ng/mL) | (h*ng/mL) | (h) | (%) | (%) |
| Powder for | OG | 250 | 3.14 | 5.00 | 6.26 | NC | NA | 0.10 | NC |
| suspension | | | (0.361) | | (2.88) | (NA) | (NA) | | |
| Powder in | Capsule | 250 | 39.8 | 3.00 | 169 | 616 | 3.73 | 1.74 | 8.64 |
| capsules | | | (43.1) | | (216) | (NA) | (NA) | | |
| Solution | Capsule | 250 | 92.4 | 2.00 | 252 | 285 | 2.87 | 3.06 | 2.97 |
| #3 | | | (65.6) | | (205) | (216) | (0.810) | | |
| Solution | OG | 250 | 59.2 | 2.50 | 240 | 107 | 1.70 | 3.30 | 1.50 |
| #3 | | | (33.6) | | (193) | (NA) | (NA) | | |
| Solution | OG | 750 | 353 | 2.00 | 1830 | 1240 | 3.86 | 8.74 | 5.47 |
| #3 | | | (207) | | (1180) | (578) | (1.63) | | |
| Solution | Capsule | 250 | 97.4 | 2.00 | 219 | 389 | 2.65 | 2.00 | 3.87 |
| #5 | | | (158) | | (277) | (333) | (1.03) | | |
| Solution | OG | 250 | 5.74 | 0.50 | 3.54 | NC | NA | 0.059 | NC |
| #5 | | | (NA) | | (NA) | (NA) | (NA) | | |
| Suspension | Capsule | 250 | 24.8 | 5.00 | 177 | 19.1^{c} | 4.37 | 0.774 | 0.268^{c} |
| S1 | | | (33.4) | | (312) | (NA) | (NA) | | |
| Suspension | OG | 250 | NA | NA | NA | NA | NA | NA | NA |
| S1 | | | (NA) | | (NA) | (NA) | (NA) | | |
| Makena | IM | 250 | 58.1 | 48.0 | 5990 | 7130 | 53.6 | NA | NA |
| ref | | | (3.72) | | (382) | (273) | (11.9) | | |

Note:
Median values are presented for Tₘₐₓ
Standard deviations are in parentheses
^{a}Calculated using geometric mean AUC₀₋ₜ values
^{b}Calculated using geometric mean AUC_{0-inf} values
^{c}For information only. Based on AUC_{0-inf} value from a single animal (5761), excluded from summary statistics due to high AUC_{%extrap} (37.9%)
NC—Could not be calculated

As assessed by mean Cₘₐₓ and AUC₀₋₂₄ values, 17-HPC was higher following capsule administration compared to oral gavage administration of PR2005 in all formulations with the exception of solution #3, for which similar Cₘₐₓ and AUC₀₋₂₄ values were observed following oral gavage and capsule dosing at a PR2005 dose level of 250 mg. 17-HPC mean Cₘₐₓ and AUC₀₋₂₄ values, increased with the increase in PR2005 dose level from 250 to 750 mg. The increases in mean Cₘₐₓ and AUC₀₋₂₄ values were generally greater than dose proportional.

The bioavailability of PR2005 relative to MAKENA® as assessed by mean AUC₀₋ₜ and AUC_{0-inf} values of 17-HPC, was lowest in the powder for aqueous suspension and solution #5 formulations when administered by oral gavage, ranging from 0.059-0.10% (based on AUC₀₋ₜ). Capsule administration of PR2005 in all formulations, resulted in higher relative bioavailability (0.774-3.06% based on AUC₀₋ₜ or 0.268-8.64% based on AUC_{0-inf}), when compared to oral gavage administration.

PR2005 in solution #3 administered by oral gavage yielded higher relative bioavailability than the other formulations (1.50-5.47% based on AUC₀₋ₜ or 3.30-8.74% based on AUC_{0-inf}). Increasing the dose level of PR2005 in solution #3 from 250 mg to 750 mg resulted in a 2.6- to 3.6-fold increase in relative bioavailability.

### Example 4—Single Dose PK of 17-HPC Liquid Filled Capsules, Phase 1 Study

Title: Single Dose Pharmacokinetics of Hydroxyprogesterone Caproate Liquid Filled Capsules. Phase 1 Study 1
Objective: Test for Dose Proportionality of 3 dose levels
Formulation: Immediate Release Gel Caps 120 mg 17-HPC per cap
Study Design: Randomized 3-way crossover

Details of the study is illustrated in Table 12.

TABLE 12

| Number of Subjects | 24 | |
|---|---|---|
| Treatment Groups | 1. | 120 mg (1 × 120 mg Soft gel caps) - fasted |
| | 2. | 360 mg (3 × 120 mg Soft gel caps) - fasted |
| | 3. | 720 mg (6 × 120 mg Soft gel caps) - fasted |
| Dosing Schedule | Single doses scheduled 5 to 7 days apart. | |
| | Random 3-way crossover allocation. | |
| | See below, Meals Composition and Meals Timing in Relation to Dose | |
| Blood Sampling | Blood specimens will be collected at the following times*: | |
| | 0 hrs (immediately prior to dose), and post dose at | |
| | 0.5, 1, 2, 3, 4, 5, 6, 7, 8 | |
| | 10, 12, 14, 16 and 24 hrs. | |
| | *Based upon expected tmax = 3 or 3.5 hrs and t½ ≤ 4 hrs,) | |
| | No sampling during 8 hour rest/sleep period) | |
| Recruitment Centers | Single PK facility with 24 hour in-house supervised residency | |
| Inclusion Criteria | 1. | Subjects must be able to understand and provide informed consent |
| | 2. | Must agree to placement of venous access for repeated blood |
| | | draws throughout the course of study |
| | 3. | Age 18 to 55 year |
| | 4. | BMI 18.5 to 32.0 kg/m2 |
| | 5. | In good general health |
| | 6. | Clinical laboratory evaluations within normal limits |
| | 7. | Normal ECG |
| | 8. | Negative for selected drugs of abuse at screening and study |
| | | check-in |
| | 9. | Negative hepatitis panel |
| | 10. | Females nonpregnant, nonlactating and either postmenopausal, |
| | | surgically sterile, or using contraceptive regimens |
| Exclusion Criteria | 1. | Inability to provide informed consent |
| | 2. | Prisoners or patients under involuntary detention (e.g, psychiatric |
| | | or substance abuse clinics) |
| | 3. | Presence of an uncontrolled, unstable clinically significant |
| | | medical condition |
| | 4. | Presence of infectious disease at screening or check-in |
| | 5. | Use of any investigational drug within 30 days or 5-half-lives |
| | | prior to entry to this study |
| | 6. | Requires the use of any concomitant medication |
| | 7. | Any clinically significant laboratory abnormality or ECG finding |
| | | at study screening or check-in. |
| | 8. | Known or suspected seizure disorder |
| | 9. | Unwilling to commit to avoidance of grapefruit or grapefruit juice |
| | | during the study period, including 30 days prior to study check-in |

| | |
|---|---|
| Pharmacokinetic | Tₘₐₓ (hr), Cₘₐₓ(μg/L), AUC_{inf} (μg · hr/L), AUCₗₐₛₜ (μg · hr/L) |
| parameters | Cl (L/hr), Vd/F (L), t½ (hr) |
| Safety Parameters | Treatment emergent adverse events |
| | Clinical Laboratory shifts toward abnormal |
| | ECG change |
| Meals. Composition | Fasted Treatments: Following an overnight fast of at least 10 hours, |
| | subjects should be administered the drug product with 240 mL (8 |
| | fluid ounces) of water. No food should be allowed for at least 4 hours |
| | post-dose. Water can be allowed as desired except for one hour before |
| | and after drug administration. Subjects should receive standardized |
| | meals scheduled at the same time in each period of the study |
| Meals - timing in | The single dose for this study will be a morning dose administered |
| relation to dose | under FDA fasted treatment conditions (after 10 hour fast with first |
| | meal withheld until 4 hours after first dose). |

### Example 5—Multiple Dose PK of 17-HPC Liquid Filled Capsules, Phase 1 Study

Title: Multiple Dose Pharmacokinetics of Hydroxyprogesterone Caproate Liquid Filled Capsules. Phase 1 Study 2
Objective: Determine Multiple Dose Kinetics of Likely Dose Regimen
Formulation: Immediate Release Gel Caps 120 mg 17-HPC per cap
Study Design: Single arm, single treatment

Details of the study is illustrated in Table 13.

TABLE 13

| Number of Subjects | 14 (12 + 2 additional subjects for possible dropout) | |
|---|---|---|
| Treatment Groups | Total Daily dose of 720 mg (360 mg BID) | |
| | (3 × 120 mg Soft gel caps, BID, every 12 hrs) | |
| Dosing Schedule | Twice daily 360 mg dose (3 × 120 mg Soft gel caps q 12 hours) for | |
| | 5.5 days. | |
| | PK monitoring through early morning of day 7. | |
| | Discharge on Day 7 | |
| | See below, Meals Composition and Meals Timing in Relation to Dose | |
| Blood Sampling | At First Dose Day 1*: | |
| | 0 hrs (immediately prior to dose), and post dose at | |
| | 1, 2, 3, 4, 5, 6, 8, 10, 12 | |
| | 12 hour sample, immediately before second dose | |
| | Trough levels during multiple dose: | |
| | Predose (Immediately before morning dose) on days 2, 3, 4, 5, 6 | |
| | Terminal elimination - With Last Dose Day 6, AM dose | |
| | Day 6: 0 hrs (immediately prior to dose) | |
| | 2, 4, 6, 8, 10, 12, 16, 20, 24 | |
| | 12 hr = before withheld PM dose day 6 | |
| | 24 hr = before withheld AM dose day 7 | |
| | Study exit procedures conducted after 24 hr sample, Day 7 | |
| Recruitment Centers | Single PK facility with 24 hour in-house supervised residency | |
| Inclusion Criteria | 1. | Subjects must be able to understand and provide informed consent |
| | 2. | Must agree to placement of venous access for repeated blood |
| | | draws throughout the course of study |
| | 3. | Age 18 to 55 year |
| | 4. | BMI 18.5 to 32.0 kg/m2 |
| | 5. | In good general health |
| | 6. | Clinical laboratory evaluations within normal limits |
| | 7. | Normal ECG |
| | 8. | Negative for selected drugs of abuse at screening and study |
| | | check-in |
| | 9. | Negative hepatitis panel |
| | 10. | Females nonpregnant, nonlactating and either postmenopausal, |
| | | surgically sterile, or using contraceptive regimens |
| Exclusion Criteria | 1. | Inability to provide informed consent |
| | 2. | Prisoners or patients under involuntary detention (e.g, psychiatric |
| | | or substance abuse clinics) |
| | 3. | Presence of an uncontrolled, unstable clinically significant |
| | | medical condition |
| | 4. | Presence of infectious disease at screening or check-in |
| | 5. | Use of any investigational drug within 30 days or 5-half-lives |
| | | prior to entry to this study |
| | 6. | Requires the use of any concomitant medication |
| | 7. | Any clinically significant laboratory abnormality or ECG finding |
| | | at study screening or check-in. |
| | 8. | Known or suspected seizure disorder |
| | 9. | Unwilling to commit to avoidance of grapefruit or grapefruit juice |
| | | during the study period, including 30 days prior to study check-in |

| | |
|---|---|
| Pharmacokinetic | First Dose: |
| parameters | Tmax (hr), Cmax(μg/L), AUClast (μg · hr/L), AUCtau (μg · hr/L), Cl/F |
| | (L/hr), Vd/F (L), |
| | Repeated Trough Levels: |
| | Mean trough serum level, Assessment for Change over Time |
| | (Accumulation ratio) |
| | Terminal Elimination; |
| | Tₘₐₓ (hr), Cₘₐₓ(μg/L), AUCₗₐₛₜ (μg · hr/L), Cl/F (L/hr), |
| | Vz/F (L), t½ (hr) |
| Safety Parameters | Treatment emergent adverse events |
| | Clinical Laboratory shifts toward abnormal |
| | ECG change |
| Meals. | Fasted Treatments: Following an overnight fast of at least 10 hours, |
| Composition | subjects should be administered the drug product with 240 mL (8 |
| | fluid ounces) of water. No food should be allowed for at least 4 hours |
| | post-dose. Water can be allowed as desired except for one hour before |
| | and after drug administration. Subjects should receive standardized |
| | meals scheduled at the same time in each period of the study |
| Meals - timing in | The morning dose on Dosing Day 1 should be administered under |
| relation to dose | FDA fasted treatment conditions (after 10 hour fast with first meal |
| | withheld until 4 hours after first dose). |
| | All subsequent doses will be administered 1 hours before |
| | standardized meal or snack. |
| | Subjects should receive standardized meals scheduled at the same |
| | time in each period of the study |

### Example 6—Single Dose PK of 17-HPC Liquid Filled Capsules, Phase 1 Study

Title: Single Dose Pharmacokinetics of Hydroxyprogesterone Caproate Liquid Filled Capsules. Phase 1 Study 3
Objective: Determine the food effect on 17-TPC liquid filled capsules
Formulation: Immediate Release Gel Caps 120 mg 17-PC per cap
Study Design: Randomized 3-way crossover

Details of the study is illustrated in Table 14.

TABLE 14

| Number of Subjects | 24 | |
|---|---|---|
| Treatment Groups | High Dose Fasted, High Dose Fed, Medium Dose Fed | |
| | 720 mg (6 × 120 mg Soft gel caps) - fasted | |
| | 720 mg (6 × 120 mg Soft gel caps) - fed | |
| | 360 mg (3 × 120 mg Soft gel caps) - fed | |
| | See below, Meals Composition and Meals Timing in Relation to Dose | |
| Dosing Schedule | Single doses scheduled 5 to 7 days apart. | |
| | Random 3-way crossover allocation. | |
| Blood Sampling | Blood specimens will be collected at the following times*: | |
| | 0 hrs (immediately prior to dose), and post dose at | |
| | 0.5, 1, 2, 3, 4, 5, 6, 7, 8 | |
| | 10, 12, 14, 16 and 24 hrs. | |
| | *Based upon expected tmax = 3 or 3.5 hrs and t½ ≤ 4 hrs,) | |
| | No sampling during 8 hour rest/sleep period) | |
| Recruitment Centers | Single PK facility with 24 hour in-house supervised residency | |
| Inclusion Criteria | 1. | Subjects must be able to understand and provide informed consent |
| | 2. | Must agree to placement of venous access for repeated blood |
| | | draws throughout the course of study |
| | 3. | Age 18 to 55 year |
| | 4. | BMI 18.5 to 32.0 kg /m2 |
| | 5. | In good general health |
| | 6. | Clinical laboratory evaluations within normal limits |
| | 7. | Normal ECG |
| | 8. | Negative for selected drugs of abuse at screening and study |
| | | check-in |
| | 9. | Negative hepatitis panel |
| | 10. | Females nonpregnant, nonlactating and either postmenopausal, |
| | | surgically sterile, or using contraceptive regimens |
| Exclusion Criteria | 1. | Inability to provide informed consent |
| | 2. | Prisoners or patients under involuntary detention (e.g, psychiatric |
| | | or substance abuse clinics) |
| | 3. | Presence of an uncontrolled, unstable clinically significant |
| | | medical condition |
| | 4. | Presence of infectious disease at screening or check-in |
| | 5. | Use of any investigational drug within 30 days or 5-half-lives |
| | | prior to entry to this study |
| | 6. | Requires the use of any concomitant medication |
| | 7. | Any clinically significant laboratory abnormality or ECG finding |
| | | at study screening or check-in. |
| | 8. | Known or suspected seizure disorder |
| | 9. | Unwilling to commit to avoidance of grapefruit or grapefruit |
| | | juice during the study period, including 30 days prior to study |
| | | check-pin |

| | |
|---|---|
| Pharmacokinetic | Tmax (hr), Cmax(μg/L), AUCinf (μg · hr/L), AUClast (μg · hr/L) |
| parameters | Cl (L/hr), Vd/F (L), t½ (hr) |
| Safety Parameters | Treatment emergent adverse events |
| | Clinical Laboratory shifts toward abnormal |
| | ECG change |
| Meals. | The meal will be an FDA high fat meal: A high-fat (approximately 50 |
| Composition | percent of total caloric content of the meal) and high-calorie |
| FDA high fat meal | (approximately 800 to 1000 calories) meal is recommended as a test |
| | meal for food-effect BA and fed BE studies. This test meal should |
| | derive approximately 150, 250, and 500-600 calories from protein, |
| | carbohydrate, and fat, respectively.4 The caloric breakdown of the |
| | test meal should be provided in the study report. |
| | An example FDA high fat would be two eggs fried in butter, two |
| | strips of bacon, two slices of toast with butter, four ounces of hash |
| | brown potatoes and eight ounces of whole milk. Substitutions in this |
| | test meal can be made as long as the meal provides a similar amount |
| | of calories from protein, carbohydrate, and fat and has comparable |
| | meal volume and viscosity. |
| Meals - timing in | Fasted Treatments: Following an overnight fast of at least 10 hours, |
| relation to dose | subjects should be administered the drug product with 240 mL (8 |
| | fluid ounces) of water. No food should be allowed for at least 4 hours |
| | post-dose. Water can be allowed as desired except for one hour before |
| | and after drug administration. Subjects should receive standardized |
| | meals scheduled at the same time in each period of the study |
| | Fed Treatments: Following an overnight fast of at least 10 hours, |
| | subjects should start the recommended meal 30 minutes prior to |
| | administration of the drug product. Study subjects should eat this |
| | meal in 30 minutes or less; however, the drug product should be |
| | administered 30 minutes after start of the meal. The drug product |
| | should be administered with 240 mL (8 fluid ounces) of water. No |
| | food should be allowed for at least 4 hours post-dose. Water can be |
| | allowed as desired except for one hour before and after drug |
| | administration. Subjects should receive standardized meals scheduled |
| | at the same time in each period of the study |

### Example 7—Phase 2 Study for the Treatment of Cryptogenic Organizing Pneumonia

Details of the study is illustrated in Table 15.

TABLE 15

| | |
|---|---|
| Protocol Number | |
| Title of Study | A randomized, multi-center, open-labelled, three-arm phase II study |
| | to assess the efficacy and safety of EG001: Arm one: EG001a 250 mg + |
| | standard of care, Arm two: EG001a 750 mg + low dose of standard |
| | of care, Arm three: standard of care in patients with Cryptogenic |
| | organizing pneumonia. |
| Phase of Trial | Phase II |
| Indications | Cryptogenic organizing Pneumonia |
| Study Objective | Primary objective: |
| | To assess if combination in EG001a + Prednisone results in overall |
| | complete recovery (CR) efficacy in COP patients compared to standard |
| | of care at two dose level. |
| | Secondary objectives: |
| | To determine if combination of EG001a + prednisone administration |
| | demonstrates additional benefit in terms of |
| | Time to response to therapy |
| | Time to complete recovery after treatments |
| | Time to disease improvement |
| | Improve Partial response rate |
| | Improve Stable response rate |
| | Reduce Relapse |
| | Exploratory objectives |
| | Change of BMI |
| Study Endpoints | Primary Endpoints: |
| | Change from the baseline of FVC |
| | Change from the baseline of in diffusion capacity for carbon monoxide |
| | (DLCO) |
| | Change from the baseline of total Lung capacity |
| | Change from the baseline of HRCT |
| | Change from the baseline of 6 minutes walk test |
| | Secondary Endpoints: |
| | Improvement in clinical symptoms at 1, 2, 3 and 4 months after |
| | treatment |
| | Change from the baseline of HRCT at 1, 2, 3, and 4 months after |
| | treatment |
| | St George's Respiratory Questionnaire(SGRQ) at 1, 2, 3, and 4 months |
| | after treatment |
| | Modified Medical Research Council (mMRC) dyspnea scale at |
| | baseline, after 1 month, after 2 month, after 3 months and after 4 |
| | months |
| | Exploratory Endpoints: |
| | Weight measurement on every follow up |
| Safety assessment | Vital sign, physical exam, Laboratory test: white blood cell (WBC) |
| | count, lactate dehydrogenase (LDH), alkaline phosphatase (ALP), C |
| | reactive protein (CRP), KL-6, surfactant protein D (SP-D), |
| | white blood cell (WBC) count, lactate dehydrogenase (LDH), alkaline |
| | phosphatase (ALP), C reactive protein (CRP), KL-6, surfactant protein |
| | D (SP-D), EKG |
| Study Design | Diagnosis: |
| | COP is diagnosed in the appropriate clinical, radiographic, and |
| | pathological setting after excluding situations associated with |
| | SOP. Abnormal chest radiograph and/or thorax high-resolution |
| | computed tomography (HRCT) ranging from multiple acinar/ |
| | nodular shadows, |
| | Biopsy, the presence of intraluminal fibrotic buds within the |
| | alveoli and alveolar ducts with or without bronchiolar |
| | involvement and infiltration of chronic inflammatory cells in |
| | the alveolar septa with preservation of the alveolar structure, 3. |
| | Negative microbiological analysis on bronchoalveolar lavage |
| | (BAL) fluid |
| | Definition |
| | Complete recovery (CR): after treatment was defined as |
| | complete or near-complete normalization of PFT and |
| | disappearance of radiologic abnormalities. Disease |
| | improvement: in general was defined as a more than 10% |
| | increase in forced vital capacity (FVC), an increase of more |
| | than 15% in diffusion capacity for carbon monoxide (DLCO), |
| | and/or a more than 10% increase in total lung capacity (TLC). |
| | Partial response (PR): Patients who showed disease |
| | improvement but did not reach CR were defined to have partial |
| | response. |
| | Disease progression (DP): was defined as a decrease in FVC of |
| | more than 10%, a decrease in DLCO of more than 15%, and/or |
| | a decrease in TLC of more than 10%. |
| | Stable disease (SD): Patients in neither the improvement nor the |
| | progression categories were defined to have stable disease. |
| | Relapse: If characteristic lung parenchymal abnormalities and |
| | compatible clinical symptoms reappeared in a patient after an |
| | initial positive response to steroid treatment, a diagnosis of |
| | disease relapse was made. |
| | Methodology: Randomized, multicenter, open-label, three arm study. |
| | This is a Phase II randomized, open-labelled, multicenter study to |
| | compare the efficacy and safety of agent EG001a IM in 750 mg + low |
| | dose prednisone (oral) treatment and EG001a IM in 250 mg + |
| | prednisone (oral) treatment and prednisone only in patients with |
| | Cryptogenic organizing Pneumonia. Eligible patients with documented |
| | appropriate clinical, radiographic, and pathological setting will be |
| | enrolled. Eligible patients will be randomized to one of the three study |
| | for 8 weeks of either receive EG001a 250 mg IM once/week, EG001a |
| | 750 mg IM once/week + prednisone or prednisone only. Approximately |
| | 30 patients (10 patients in each cohort) will be randomized in a 1:1:1 |
| | ratio (low dose EG001a + Prednisone; high dose EG001a + Prednisone |
| | and Prednisone only) in this study. The stratification factors of |
| | randomization will be (i) sex (male vs. female); (ii) smoking status |
| | (never vs. current/former); and (iii) ECOG performance status (PS = 0 |
| | vs. PS = 1). This study consists of three phases: (i) screening and |
| | randomization; (ii) treatment; and (iii) follow up. During the screening, |
| | each potential subject will provide informed consent prior to starting |
| | any study specific procedures. The randomization of subjects to study |
| | groups (Arm one: 250 mg EG001a + Prednisone; Arm two: 750 mg |
| | EG001a + Prednisone; and Arm three Prednisone only) will be |
| | performed centrally by an Interactive Web-Response System (IWRS) |
| | using a randomization scheme that will be reviewed and approved by |
| | an independent statistician. During the treatment period, randomized |
| | subjects will be provided the treatment and assessment according to the |
| | protocol. Follow up: includes at 1, 2, 3, and 4 months of therapy follow |
| | up. Patient randomized into Arm one group will receive a EG001a 250 |
| | mg weekly IM + Prednisone oral 0.75 mg/kg/day for 4 weeks, and |
| | EG001a 250 mg weekly IM + Prednisone 0.5 mg/kg/day for 4 weeks, |
| | followed by prednisone only 20 mg/day for 4 weeks, 10 mg/day for 6 |
| | weeks, and finally 5 mg/day for 6 weeks (following standardized |
| | GERM“O”P protocol). Patient randomized into Arm two group will |
| | receive EG001a 750 mg weekly IM + Prednisone oral 0.5 mg/kg/day for |
| | 4 weeks, and EG001a 750 mg weekly IM + prednisone 20 mg/day for |
| | 4 weeks, followed by prednisone only 10 mg/day for 6 weeks, and |
| | finally 5 mg/day for 6 weeks Prednisone group will receive a dose of |
| | 0.75 mg/kg/day prednisone for 4 weeks, 0.5 mg/kg/day for 4 weeks, |
| | 20 mg/day for 4 weeks, 10 mg/day for 6 weeks, and finally 5 mg/day for |
| | 6 weeks (following standardized GERM“O”P protocol). Patients will |
| | continue their randomized treatment until treatment discontinuation or |
| | study withdrawal. In the event patients experience documented disease |
| | recovery, the physician may early taper prednisone treatment in |
| | accordance with their clinical judgement. All radiographic assessments |
| | will be carried out in accordance with Fleischner society guideline |
| | (2018) and are to be performed at 1, 2, 3 and 4 months relative to first |
| | dose of study drug, and will be repeated until objective disease relapse |
| | or progression or as per standard practice post progression. Patients |
| | will be followed at 1, 2, 3 and 4 months. |
| Treatment Duration | 2-4 months |
| and End of Study | |
| Study Centers | Multiple |
| Study Treatment | Study drug: EG001a |
| | Strength: 250 mg, 750 mg |
| | Route of administration: EG001a 250 mg or 750 mg IM weekly |
| | Duration of Treatment = 8 consecutive weeks of dosing |
| | Control drug: Placebo |
| Eligibility Criteria | Inclusion criteria |
| | Patients between 18 and 70 years |
| | Parenchymal pulmonary involvement at HRCT and one of the |
| | follows: physiologic abnormalities on pulmonary function |
| | testing and/or respiratory symptoms, and/or dyspnea, after |
| | excluding situations associated with SOP |
| | Exclusion criteria |
| | Unable to understand protocol and to sign informed consent or |
| | not suitable candidate to comply with the requirements of this |
| | study, in the opinion of the investigator |
| | Cardiac and neurological sarcoidosis or any other organ |
| | involvement |
| | End stage lung disease at high-resolution computed tomography |
| | (HRCT) |
| | Secondary OP induced by infections, drugs, or collagen |
| | vascular diseases |
| | Spontaneous improvement without treatment |
| | Clinical evidence of active infection |
| | Documented exposure to beryllium |
| | Patients with Forced Expiratory Volume at one second (FEV1) |
| | changes after salbutamol inhalation ≥20% |
| | Comorbidity: advanced liver cirrhosis or abnormal liver |
| | function, unstable cardiac disease, moderate to severe renal |
| | insufficiency, poorly controlled diabetes |
| | Pregnancy or lactation |
| | A tuberculin skin test (5 I.U.) more than 5 mm |
| | Psoriasis |
| | Homozygous glucose-6-phosphatase deficiency |
| | Concomitant therapies: any patient enrolled in the study must |
| | be off all prohibited medications at least 4 weeks before |
| | screening. Once patients completed the washout period, they |
| | may enter the screening period that may last up to 30 days |
| | Previous therapies: any patient enrolled must be off all |
| | medications for COP at least 4 weeks before screening. |
| Sample Size | 10 for each group |
| Statistical Analysis | Continuous data for normal distribution are expressed as mean ± |
| | standard deviation (SD). A p < 0.05 was considered as significant. In |
| | descriptive statistics, frequency and percentage were used for discrete |
| | data, and mean ± SD were used for continuous variables. The normality |
| | test was performed by the Kolmogorov-Smirnov and Shapiro-Wilk |
| | methods. The Mann-Whitney U test and t-test were used to compare |
| | the differences between the groups. |

### Example 8—a Phase I, Randomized, Cross-Over, Single-Center, Single Dose Fasted Pharmacokinetics of Ascending Doses of Hydroxyprogesterone Caproate (17-HPC) Oral Softgel Capsule (EGHPCP01) with Comparison to Intramuscular (IM) Injection in Healthy Volunteers

### Objectives:

To evaluate dose proportionality of HPC across 4 ascending dose levels under fasting conditions in healthy adult subjects;
To compare Oral vs IM pharmacokinetics of HPC under fasting conditions in healthy adult subjects;
To assess the safety and tolerability of HPC across four ascending dose levels under fasting conditions in healthy adult subjects; and
To evaluate the potential of Benzyl Benzoate exposure following single oral dose of HPC oral softgel capsules.

### Endpoints

PK parameters include, but not limited to, Tₘₐₓ (hr), Cₘₐₓ(μg/L), AUC_{inf} (μg·hr/L), AUCₗₐₛₜ (μg·hr/L), Cl (L/hr), Vd/F (L), t_{1/2} (hr);
Safety and tolerability assessment by monitoring adverse events (AEs), vital signs, and electrocardiograms (ECGs), assessing clinical safety laboratory values, and performing physical examinations;
Plasma PK parameters for Benzyl Benzoate and its metabolites benzyl alcohol and benzoic acid; and

The amount conjugates of benzoic acid (hippuric acid and the glucuronide of benzoic acid) in 10-hour urine samples.

Table 18 illustrates the dose level and dosing requirements of this study.

TABLE 18

| | | Dose | | | Number of | |
|---|---|---|---|---|---|---|
| Investigational | | level | | | capsule(s)/injection(s) | Dietary |
| Products | Treatment | (mg) | Strength | | required | state |
| Study drug | A | 120 | 120 | mg | 1 × 120 mg capsule | Fasted |
| Study drug | B | 240 | 120 | mg | 2 × 120 mg capsules | Fasted |
| Study drug | C | 480 | 120 | mg | 4 × 120 mg capsules | Fasted |
| Study drug | D | 720 | 120 | mg | 6 × 120 mg capsules | Fasted |
| Comparator | Low-dose | 250 | 250 | mg/ml | 250 mg/1 mL × 1 IM | Fasted |
| | | | | | Injection | |
| Comparator | High-dose | 1000 | 250 | mg/ml | 500 mg/2 mL × 2 IM | Fasted |

Injections

Twenty-four subjects were recruited for this study, including 16 who received crossover oral doses, 4 received low-dose IM injections, and 4 received high-dose IM injection. Four 17-HPC oral groups and two IM (intramuscular) injection group were carried out in this study. Subjects in the 17-HPC oral groups were randomized (1:1:1:1) to receive 1 of the 4 treatment sequences: ADBC, BACD, CBDA, and DCAB. Each sequence included 4 study drug treatments, i.e., 120 mg (Treatment A), 240 mg (Treatment B), 480 mg (Treatment C), and 720 mg (Treatment D), with a 7-day washout period between each treatment. Single-dose IM injections (250 mg and 1000 mg) were given in 2 parallel groups because of long duration of washout.

### Inclusion Criteria:

Subjects who met all of the following inclusion criteria participated in this study:
1. Adult, male and female volunteers, 18 to 55 years of age, inclusive, at first Check-In Visit
2. Body mass index (BMI)≥18.5 to ≤32 kg/m² at Screening (calculated as a function of measured height and weight according to the formula, BMI=kg/m² where m² is height in meters squared.).
3. All female subjects must be nonpregnant, nonlactating and either postmenopausal, surgically sterile, or using contraceptive regimens more than 3 months. All females must have a negative serum pregnancy test at Screening and Check-in Visit. Effective methods of contraception include a dual method of contraception: condom with spermicide in conjunction with use of an intrauterine device (IUD), condom with spermicide in conjunction with use of a diaphragm, condom with birth control patch or vaginal ring, or condom with oral, injectable, or implanted contraceptive. Surgical sterility is documented through documented: hysterectomy, partial hysterectomy, bilateral oophorectomy, or bilateral tubal ligation at least 6 months prior to Screening. Postmenopausal sterility is documented by absence of menses for at least 12 months prior to Screening plus serum FSH≥40 mIU/mL and estradiol<30 pg/mL at screening.
4. Male subjects, if sexually active with a female partner of child-bearing potential, must be vasectomized or agree to take appropriate precautions to prevent conception, including practicing an effective method of contraception, and must not donate sperm from screening through 12 weeks following administration of the last dose of study medication. Effective methods of contraception include a dual method of contraception: condom with spermicide in conjunction with use of an intrauterine device (IUD), condom with spermicide in conjunction with use of a diaphragm, condom with birth control patch or vaginal ring, or condom with oral, injectable, or implanted contraceptive.
5. Medically healthy on the basis of medical history, and physical examination (including but not limited to an evaluation of the cardiovascular, gastrointestinal, respiratory, and central nervous systems), as determined by the Investigator at Screening and each Check-In Visit
6. Medically healthy based on the absence of clinically significant abnormal vital sign measurements, clinical laboratory test results (especially tests for renal and hepatic function) as determined by the Investigator at Screening and each Check-In Visit
7. Subjects may include users of tobacco and other nicotine products if their frequency of use allows abstinence throughout the in-house portions of the study. For a period of at least 6 months prior to Screening, all subjects must have been free from excessive alcohol intake or regular use of recreational drugs regulated as illegal in all 50 states of the USA. Past history of marijuana is not excluded except during the in-house portions of this study.
8. Ability to understand and willingness to sign a written informed consent form (The consent form must be signed by the subject prior to any study-specific procedures.)
9. Willingness and ability to receive placement of venous access for repeated blood draws throughout the course of study, and comply with study procedures and follow-up examination.

### Exclusion Criteria

Subjects who met any of the following exclusion criteria were not enrolled in this study.
1. Females who are pregnant, lactating, or likely to become pregnant during the study
2. History and/or recent evidence within 6 months prior to Screening of alcohol or drug/substance abuse disorder
3. Subjects with a history of hypersensitivity to Hydroxyprogesterone Caproate or any component of study medication
4. History of clinically significant allergies including drug allergies or allergic bronchial asthma or related bronchospastic conditions
5. Subjects who have history of unexplained syncope or fainting or a condition that predisposes them to syncope, such as hypotension, orthostatic hypotension, bradycardia or dehydration
6. Subjects determined by the Investigator to have any medical condition that could jeopardize their health or prejudice study results (e.g., history of surgery of the gastrointestinal tract, which may interfere with absorption, except for appendectomy);
7. Subjects who have used P-gp and/or CYP 450 hepatic microsomal enzyme-inducing or inhibiting drugs (e.g., propafenone, voriconazole, fluconazole, cimetidine) within 30 days of first dosing
8. Subjects with a history or presence of significant cardiovascular, pulmonary, hepatic, gallbladder or biliary tract, renal, hematologic, gastrointestinal, endocrine, immunologic, dermatologic, neurologic, psychiatric disease, or active sexually transmitted disease to include:
   Current or history of thrombosis or thromboembolic disorders
   Known or suspected breast cancer, other hormone-sensitive cancer, or history of these conditions
   Undiagnosed abnormal vaginal bleeding unrelated to pregnancy
   Cholestatic jaundice of pregnancy
   Liver tumors, benign or malignant, or active liver disease
   Uncontrolled hypertension
9. History or evidence of acute or chronic respiratory disorders, including but not limited to chronic obstructive pulmonary disease (COPD) or asthma
10. History or clinical evidence of achlorhydria, severe gastrointestinal disease, particularly diarrhea or other conditions affecting gastrointestinal mobility or absorption
11. History or presence of significant cardiovascular abnormalities, including without limitation, severe bradycardia, sick sinus syndrome, second- or third-degree atrial ventricular block, long QT syndrome, cardiogenic shock, and decompensated heart failure
12. History or presence of pro-arrhythmic conditions, including a marked baseline prolongation of QTc interval (i.e., repeated demonstration of a QTc interval >450 milliseconds) or a history of additional significant risk factors for torsade de pointes (e.g., family history of long QT syndrome), including any evidence of QTc prolongation at screening
13. Subjects with estimated glomerular filtration rate (eGFR)<90 mL/min/1.73 m²; subjects with slightly lower values may be included upon agreement between the sponsor medical representative and the Principal Investigator at screening and Day −1.
14. Subjects who have fasting triglyceride (TG)>ULN
15. Subjects meeting any of the following laboratory criteria will be excluded:
   Screening total bilirubin>1.3 mg/dL; subjects with a documented history of Gilbert's syndrome can be enrolled if the direct bilirubin is within normal reference range.
   Screening alanine aminotransferase (ALT), aspartate aminotransferase (AST), alkaline phosphatase (ALP)>upper limits of normal (ULN)
   Screening platelets<150,000/mcL
   Screening INR>1.2
16. Subjects who test positive at screening for human immunodeficiency virus (HIV), Hepatitis B surface antigen (HbsAg), or Hepatitis C virus (HCV) antibody
17. Subjects who test positive at Screening and/or admission (Day −1) for alcohol and/or drugs of abuse
18. Subjects who donated ≥500 mL of blood within 56 days prior to the first study period or ≥50 mL and ≤499 mL of blood within 30 days prior to the first study period
19. Subject who is unable to refrain from or anticipated the use of any medication, including prescription and non-prescription drugs or herbal remedies (such as St. John's Wort [hypericum perforatum]), or grapefruits, grapefruit juice, blood oranges, apples and mulberry juice as well as vegetables from the mustard green family (e.g., kale, broccoli, watercress, collard greens, kohlrabi, Brussels sprouts, and mustard greens) beginning approximately 2 weeks prior to administration of the initial dose of study drug, throughout the study, until the poststudy visit
20. Subject who is unable to refrain from excessive alcohol consumption within one week prior to study start throughout the study, until the final study visit. Moderate alcohol consumption is defined as 1 standard drink per day for women and 2 drinks per day for men; whereby 1 standard drink is equivalent to: 12 oz beer (5% alcohol); 5 ounces of wine (12% alcohol), and 1.5 ounces of 80 proof (40% alcohol). Excessive consumption would be considered consistently in excess of twice the moderate recommendation.
21. Subject who consumes excessive amounts of caffeine for one month prior to the study drug administration, defined as greater than 6 servings (1 serving is approximately equivalent to 120 mg of caffeine) of coffee, tea, cola, or other caffeinated beverages per day
22. Subjects who donated plasma (e.g., plasmapheresis) within 14 days prior to first study period; Subjects will be advised not to donate plasma for 14 days after completing the study
23. Subjects who have participated in another clinical trial which required blood draws within 30 days prior to the first study drug dosing

### Safety Parameters

Safety and tolerability were assessed by monitoring the following parameters, including the incidence of reported adverse events, physical examinations (including body weight), vital signs, clinical laboratory tests (Blood coagulation, hematology, serum chemistry, and urinalysis), and 12-lead electrocardiograms.

### Adverse Events

Vital signs (respiratory rate, blood pressure and pulse rate from a sitting position, and body temperature)

### Physical Examination

Laboratory examination includes: Blood coagulation, hematology, urinalysis, serum chemistry
Resting 12-lead electrocardiograms (ECG) assessment will include overall interpretation, PR interval, QRS duration, RR, QT, and QTcF intervals.

### Pharmacokinetic Evaluation

The main pharmacokinetics parameters included Tₘₐₓ (hr), Cₘₐₓ(μg/L), AUC_{inf} (μg·hr/L), AUCₗₐₛₜ (μg·hr/L), Cl (L/hr), Vd/F (L), and t_{1/2} (hr).

### PK Sampling Scheme

The Four Oral Groups on Days 1 and 2 of each period
Day 1: at pre-dose (within 30 minutes), 0.5 h, 1 h, 1.5 h, 2 h, 2.5 h, 3 h, 3.5 h, 4 h, 4.5 h, 5 h, 5.5 h, 6 h, 7 h, 8 h, 10 h, and 12 h post-dose
Day 2: at 18 h, 20 h, and 24 h post-dose

The Two IM Injection Groups over the 28 Days:
Day 1: at pre-dose (within 30 minutes), 4.5 h, 6 h, and 12 h post-dose
Day 2: at 18 h, 24 h, 30 h, and 36 h post-dose
Day 3: at 48 h post-dose

During outpatient visits on Days 4, 5, 6, 7, 9, 14, 21, and 28 with 24-hour interval

The PK sampling schedule and/or washout period duration may be modified so that the PK profile of the proposed HPC oral softgel capsule could be adequately characterized and to ensure that HPC is completely washed out between treatments.

Table 21 illustrates the results from the study.

TABLE 21
Area Under Curve (AUC)
Hydroxyprogesterone Caproate
Study EG009A -1.1
Phase 1 Ascending Dose Oral and IM PK Study

| | | | Geometric | Arithmetic |
|---|---|---|---|---|
| | Dose | Observation | Mean | Mean |
| Treatment | Mg | Period | AUC_{inf} | AUC_{inf} |
| A (120 mg) | 120 | 24 hrs | 7.921 | 10.195 |
| B (240 mg) | 240 | 24 hrs | 19.468 | 22.84 |
| C (480 mg) | 480 | 24 hrs | 45.311 | 53.463 |
| D (720 mg) | 720 | 24 hrs | 65.214 | 71.22 |
| IM 250 | 250 | 648 hrs | 2085.017 | 2139.766 |
| IM1000 | 1000 | 648 hrs | 8258.536 | 8451.364 |

Solvent system: 9:1 benzyl benzoate:polyoxy-35-castor oil
Oral Dosage Form: Liquid filled gelcaps 120 mg HPC/each.
IM Dosage form: 250 mg/1 mL US Marketed Product

Also see FIG. 8. The two arrows represent unexpected multiple absorption peaks.

As shown in Table 21, whether analyzed using Geometric Means or Arithmetic means, the four ascending single doses ranging from 120 mg to 720 mg demonstrated linear dose proportionality. Since the AUC is linear across the dose ranges of 120 mg to 720 mg, this finding teaches that an oral dose of about 800 mg given once daily can rival the daily AUC contribution of a 250 mg IM injection administered once. Similarly, a 4.5×720 mg dose can rival the AUC contribution of a single dose of 1000 mg IM injection.

### Example 9—Additional Exemplary 17-HPC Formulations

Table 22 and Table 23 provide additional exemplary 17-HPC formulations used or contemplated for this invention.

TABLE 22

| | | | 17-HPC | | |
|---|---|---|---|---|---|
| | | % | concentration | Dispersion | Dispersion |
| Trial # | Excipients | (w/w)^{a} | (mg/g)^{b} | aspect | results |
| 1 | Propylene glycol | 70 | 100 | Milky | 17-HPC |
| | monocaprylate | | | dispersion | precipitation |
| | PEG 35 castor oil | 30 | | | observed after |
| | | | | | 24 h |
| 2 | Propylene glycol | 70 | 120 | Milky | 17-HPC |
| | monocaprylate | | | dispersion | precipitation |
| | PEG 35 castor oil | 30 | | | observed after |
| | | | | | 24 h |
| 3 | Propylene glycol | 70 | 150 | Milky | Limited 17- |
| | monocaprylate | | | dispersion | HPC |
| | PEG 35 castor oil | 30 | | | precipitation |
| | | | | | observed after |
| | | | | | 24 h |
| 4 | Propylene glycol | 63 | 150 | Milky | 17-HPC |
| | monocaprylate | | | dispersion | precipitation |
| | PEG 35 castor oil | 27 | | | observed after |
| | Benzyl benzoate | 10 | | | 24 h |

^{a}The percentage w/w listed indicates the weight percentage of the solvent system. For example, 70% w/w of propylene glycol monocaprylate and 30% w/w of PEG 35 castor oil constitute 100% w/w of the solvent system. In a composition, the % w/w of the solvent system is 75% w/w.
^{b}The concentration of the 17-HPC constitutes 25% w/w, with the solvent system constitutes the remaining 75% w/w.

TABLE 23

| | | | 17-HPC |
|---|---|---|---|
| | | | concentration |
| Trial # | Excipients | % (w/w)^{a} | (mg/g)^{b} |
| 1 | Propylene glycol monolaurate | 70 | 120 |
| | PEG 35 castor oil | 30 | |
| 2 | Propylene glycol monolaurate | 70 | 150 |
| | PEG 35 castor oil | 30 | |
| 3 | Glyceryl monocaprylate | 70 | 120 |
| | PEG 35 castor oil | 30 | |
| 4 | Glyceryl monocaprylate | 70 | 150 |
| | PEG 35 castor oil | 30 | |
| 5 | Propylene glycol monolaurate | 63 | 120 |
| | PEG 35 castor oil | 27 | |
| | Benzyl benzoate | 10 | |
| 6 | Propylene glycol monolaurate | 63 | 150 |
| | PEG 35 castor oil | 27 | |
| | Benzyl benzoate | 10 | |
| 7 | Glyceryl monocaprylate | 63 | 120 |
| | PEG 35 castor oil | 27 | |
| | Benzyl benzoate | 10 | |
| 8 | Glyceryl monocaprylate | 63 | 150 |
| | PEG 35 castor oil | 27 | |
| | Benzyl benzoate | 10 | |

^{a}The percentage w/w listed indicates the weight percentage of the solvent system. For example, 70% w/w of propylene glycol monocaprylate and 30% w/w of PEG 35 castor oil constitute 100% w/w of the solvent system. In a composition, the % w/w of the solvent system is 75% w/w.
^{b}The concentration of the 17-HPC constitutes 25% w/w, with the solvent system constitutes the remaining 75% w/w.

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

1. A method of treating a respiratory disease or condition associated with or induced by a pathogen in a subject in need thereof, comprising administering to the subject a composition comprising 17-alpha hydroxyprogesterone caproate (17-HPC), one or more solubilizing agents, and one or more lipophilic agents to treat the respiratory disease in the subject.

2. The method of claim 1, wherein the pathogen is a virus.

3. The method of claim 1, wherein the virus is a coronavirus, optionally an alpha-type coronavirus or a beta-type coronavirus, further optionally 229E, NL63, OC43, HKU1, MERS-CoV, SARS-CoV, or SARS-CoV-2.

4. The method of claim 1, wherein the virus is an influenza virus, cytomegalovirus, Epstein-Barr virus, variola virus, Ebola, dengue, Measles virus, mumps virus, or rubella virus.

5. The method of claim 1, wherein the pathogen is a bacterium, a fungus, a protozoan, or a parasite.

6. The method of claim 5, wherein the respiratory disease is caused by group A streptococcus (GAS), optionally comprising Streptococcus pyogenes or Streptococcus dysgalactiae.

7. The method of claim 1, wherein the subject has an elevated level of one or more mediators associated with cytokine storm.

8. The method of claim 1, wherein the respiratory disease or condition is a lower respiratory disease or condition.

9. The method of claim 1, wherein the respiratory disease or condition is pneumonia, optionally a SARS-CoV-2 induced pneumonia.

10. (canceled)

11. A method of treating a cytokine release syndrome (CRS) in a subject in need thereof, comprising administering to the subject a composition comprising 17-alpha hydroxyprogesterone caproate (17-HPC), one or more solubilizing agents, and one or more lipophilic agents to treat the cytokine release syndrome in the subject.

12. A method of modulating the level of one or more mediators of cytokine storm in a subject in need thereof, comprising administering to the subject a composition comprising 17-alpha hydroxyprogesterone caproate (17-HPC), one or more solubilizing agents, and one or more lipophilic agents to modulate the level of the one or more mediators.

13. A method of treating a subject selected for therapy, comprising administering to the subject having an elevated level of a mediator associated with cytokine storm as compared to a predetermined level of the mediator a composition comprising 17-alpha hydroxyprogesterone caproate (17-HPC), one or more solubilizing agents, and one or more lipophilic agents.

14. The method of claim 13, wherein the subject is selected for the therapy by a method comprising detecting an elevated level of a mediator associated with cytokine storm in a sample isolated from the subject.

15. The method of claim 11, wherein the subject has an elevated level of one or more mediators associated with cytokine storm as compared to a predetermined level.

16.-18. (canceled)

19. The method of claim 1, wherein the one or more mediators comprise:
a Type I IFN, a Type II IFN, or a Type III IFN;
IL-1, IL-2, IL-4, IL-6, IL-17, TNF-α, or a combination thereof;
IL-1RA, IL-2R, or a combination hereof,
IL-2, IL-7, G-CSF, CXCL 10, MCP-1, MIP-1α, TNF-α, IL-6, or a combination thereof;
IL-2R, IL-6, or a combination thereof;
IL-1B, IFN-γ, IP-10, MCP-1, or a combination thereof;
IL-4, IL-10, or a combination thereof;
G-CSF, IP-10, MCP-1, MIP1A, TNF-α, or a combination thereof;
IL-2, IL-7, IL-10, G-CSF, IP10, MCP1, MIP1A, TNF-α, or a combination thereof;
IL-6;
D-dimers; or
IFN-γ, IL-1RA, IL-2RA, IL-6, IL-10, IL-18, HGF, MCP-3, MIG, M-CSF, G-CSF, MIG-1a, CTACK, IP-10, or a combination thereof.

20. The method of claim 1, wherein the level is a serum level or an expression level.

21.-23. (canceled)

24. The method of claim 1, wherein 17-HPC decreases the level of IL-1, IL-2, IL-4, IL-6, IL-17, TNF-α, or a combination thereof.

25. The method of claim 1, wherein the subject has an elevated level of C-reactive protein (CRP), ferritin, procalcitonin, neopterin, S100 proteins, ADA2, or CD163, or a combination thereof.

26.-35. (canceled)

36. The method of claim 1, wherein the composition comprises a range of 17-HPC, optionally:
selected from: about 12% w/w to about 75% w/w, about 12% w/w to about 74% w/w, about 12% w/w to about 63% w/w, about 12% w/w to about 36% w/w, about 12% w/w to about 30% w/w, about 12% w/w to about 25% w/w, about 12% w/w to about 24% w/w, about 24% w/w to about 75% w/w, about 24% w/w to about 74% w/w, about 24% w/w to about 63% w/w, about 24% w/w to about 36% w/w, about 24% w/w to about 30% w/w, about 25% w/w to about 75% w/w, about 25% w/w to about 74% w/w, about 25% w/w to about 63% w/w, about 25% w/w to about 36% w/w, about 25% w/w to about 30% w/w, about 36% w/w to about 75% w/w, about 36% w/w to about 74% w/w, or about 36% w/w to about 63% w/w;
selected from: about 12% w/w to about 36% w/w, about 12% w/w to about 25% w/w, about 12% w/w to about 24% w/w, about 24% w/w to about 36% w/w, or about 25% w/w to about 36% w/w; or
selected from: about 6% w/v to about 36% w/v, about 6% w/v to about 24% w/v, about 6% w/v to about 18% w/v, about 12% w/v to about 36% w/v, about 12% w/v to about 18% w/v, about 12% w/v to about 24% w/v, about 18% w/v to about 36% w/v, or about 24% w/v to about 36% w/v.

37.-50. (canceled)

51. The method of claim 1, wherein the composition is:
a solution;
formulated for oral administration;
formulated as an oral capsule, optionally a soft gelatin capsule; or
formulated as an injection.

52.-58. (canceled)
